(19) United States
(12) Patent Application Publication
(10) Pub. No.: US 2026/0218240 A1
(43) Pub. Date: Jul. 30, 2026

(54) SHUFFLE CASSETTES AND GENOME SHUFFLE SEQUENCING

(71) Applicant: University of Washington, Seattle, WA (US)

(72) Inventors: Sudarshan Pinglay, Seattle, WA (US); Jay Shendure, Seattle, WA (US); Riza M. Daza, Seattle, WA (US); Jean-Benoit Lalanne, Seattle, WA (US); Xiaoyi Li, Seattle, WA (US); David Lee, Seattle, WA (US)

(73) Assignee: University of Washington, Seattle, WA (US)

(21) Appl. No.: 19/452,093

(22) Filed: Jan. 16, 2026

Related U.S. Application Data
(60) Provisional application No. 63/746,618, filed on Jan. 17, 2025.

(51) Int. Cl. C12N 15/90; C12N 15/85

(52) U.S. Cl. CPC C12N 15/90; C12N 15/85; C12N 2800/30

(57) ABSTRACT

Provided herein are compositions and methods for multiplex generation, mapping, and characterization of structural variants in a host cell genome. A shuffle cassette includes a recombination site flanked by a pair of unique barcodes, a pair of primer binding sites, and a pair of promoters. Libraries of shuffle cassettes are integrated throughout a host cell genome, and their genomic positions are mapped by transcribing flanking genomic sequences via the promoters and associating barcode pairs with specific genomic coordinates. Upon introduction of a recombinase, recombination between integrated shuffle cassettes generates structural variants including deletions, inversions, translocations, and extrachromosomal circular DNAs. Novel barcode combinations arising from recombination events are detected to identify the class and breakpoints of each structural variant at base-pair resolution without whole-genome sequencing. The methods further enable co-capture of structural variant identity with single-cell transcriptomes, linking specific structural variants to gene expression phenotypes.

### CROSS-REFERENCE TO RELATED APPLICATION

This application claims priority to and the benefit of the earlier filing date of U.S. Provisional Patent Application No. 63/746,618 filed on Jan. 17, 2025, which is incorporated herein by reference in its entirety as if fully set forth herein.

### STATEMENT REGARDING FEDERALLY SPONSORED RESEARCH OR DEVELOPMENT

This invention was made with government support under Grant Nos. DP50D036167 and R01HG010632 and RM1HG009491, awarded by the National Institutes of Health (NIH). The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3K17948.XML. The file is 4,535 bytes, was created on Jan. 14, 2026, and is being submitted electronically via Patent Center.

### BACKGROUND

Major classes of human genetic variation include single nucleotide variants (SNVs), indels, simple sequence repeat variants, and genomic structural variations (e.g., deletions, insertions, inversions, duplications, and chromosomal translocations). Historically, genomic structural variations have been at a distinct disadvantage in terms of an ability to study their functional consequences, as compared to SNVs or indels.

### BRIEF DESCRIPTION OF THE FIGURES

The patent or application file contains at least one drawing executed in color. Copies of this patent or patent application publication with color drawing(s) will be provided by the Office upon request and payment of the necessary fee. Applicant considers the color versions of the drawings as part of the original submission and reserves the right to present color images of the drawings in later proceedings.

FIGS. 1A-1E. Schematic of Genome-Shuffle-seq for the pooled construction and efficient characterization of rearranged mammalian genomes at single-cell resolution. FIG. 1A. Arrays of integrated recombinase sites can be recombined by site-specific recombinases to yield three classes of SVs. FIG. 1B. Schematic of the shuffle cassette, which contains a recombinase site flanked by unique 20N barcodes, capture sequences for scRNA-seq (CS1, CS2), and phage polymerase promoters that are inert in live mammalian cells but activated upon in vitro (IVT) or in situ (IST) transcription with T7 polymerase. FIG. 1C. Workflow of a Genome-Shuffle-seq experiment.

FIG. 1D. Shuffle cassette insertion sites can be mapped by sequencing T7-derived transcripts from IVT or IST and associating a pair of unique barcodes (numbered 1-8 in schematic) to a genomic location. Allele-specific integration sites can be determined in hybrid cells such as BL6XCAST mouse embryonic stem cells (mESCs). The stars indicate variants between the BL6 and Castaneus haplotypes in genomic DNA flanking an example integration site. FIG. 1E. Induced SVs can be inferred by novel barcode combinations that are only observed in amplicons or scRNA-seq data from cells that have been exposed to recombinase. As the genomic coordinates of the parental barcodes are known from IVT-based mapping of their locations in the parental cell population, the identity of the barcodes making up each novel combination is sufficient to infer both the class (deletion, inversion, translocation) as well as the precise genomic coordinates involved in each induced SV.

FIGS. 2A-2D. Schematic of sequencing library construction strategies for amplicon-seq and IVT-seq. FIG. 2A. Schematic of the shuffle cassette bearing the IoxPsym site (diamond) flanked by two barcodes (BC1, BC2). Two 10× Genomics capture sequences (CS1, CS2) serve as binding sites for PCR primers for sequencing library construction. Only amplicons generated using one unique molecular identifier (UMI)-containing primer and one non-UMI primer can cluster and be successfully sequenced on an Illumina flow cell due to the sequencing adapters they encode. FIG. 2B. After IVT, transcripts are generated from both the top and bottom strand T7 promoters, and are expected to contain both BC1 and BC2 as well as adjacent genomic sequences from one side of the integrated shuffle cassette. Reverse transcription (RT) is performed with a primer containing 8 random bases at its 3′ end. PCR is performed with a UMI-containing primer and one primer annealing to the constant sequence in the RT primer to yield the final sequencing library. FIG. 2C and FIG. 2D. Recombination between two insertions in cis can lead to an inversion or deletion with shuffle cassettes containing either the same or different capture sequences. Theoretically, PCR products from a cassette with the same CS should amplify and cluster on an Illumina flowcell when libraries are generated using the 4 primers.

FIGS. 3A-3D. Allele-specific mapping of shuffle cassette insertions. FIG. 3A. Insertion sites detected across chromosome 1 in a bottlenecked population of BL6xCAST mESCs, colored by allele. Insets depict pileups of sequencing reads from T7 transcripts for exemplary integrations to the CAST (left) or BL6 (right) haplotype. Alleles are distinguished by the presence of known variants between them. FIG. 3B. Number of insertion sites with unique barcodes (y-axis) across chromosomes of varying lengths (X-axis). The dotted line indicates a linear regression model fit, and the shaded gray areas indicate the 95% confidence interval. FIG. 3C. Pie chart depicting the distribution of assignments to BL6 or CAST alleles for shuffle cassettes whose genomic coordinates were mapped with high confidence. FIG. 3D. UpSet plot of the intersection of shuffle cassette integration sites with genomic features.

FIG. 4. Allele-specific insertion sites across the chromosomes. Insertion sites across the chromosomes for shuffle cassettes whose genomic coordinates were mapped with high confidence, colored by allele. Inconclusive indicates that there is conflicting evidence for the insertion allele, while noVariant denotes those insertions that were unassigned due to a lack of reads that overlap with a known variant between the BL6 and CAST genomes.

FIGS. 5A-5G. Multiplex induction and efficient genotyping of large-scale rearrangements throughout a mammalian genome. FIG. 5A. Experimental schematic. FIG. 5B. Number of novel (i.e. non-parental) barcode combinations at ≥2 UMI detected in each condition from amplicon-seq data. Different colors indicate those rearranged barcode combinations found in one technical replicate or both within each condition. FIG. 5C. Pie chart showing the distribution of SV types that are detected in both technical replicates of a Cre transfection sample. SVs detected in multiple conditions are counted independently. FIG. 5D. Schematic of approach for validation of SV calls using matched IVT-seq data from the same sample (top). The proportion of each SV type that is supported by at least one read in the IVT-seq data is depicted below. FIG. 5E. Circos plots of the unique set of SVs that are shared between technical replicates of each sample. SVs detected in multiple conditions are counted once. FIG. 5F. Scatter plot of rearrangement size (y-axis) vs. mean read count (X-axis) for deletions and inversions detected at day 3. Pearson correlation is calculated between the log 10 values of the two metrics. FIG. 5G. Violin plots depicting the distribution of read counts for deletions, inversions, and translocations detected at day 3. Inset within each violin plot is a box plot of the distribution with the median value depicted as a white line, the length of the box depicting the interquartile range, and the whiskers depicting the extent of the distribution. P-values are calculated using the non-parametric Mann-Whitney U test.

FIGS. 6A-6G. Characteristics of the complete set of rearrangements detected in bulk by amplicon-seq at 72 h post-Cre transfection. FIG. 6A. Log 2 ratio of total reads that contain rearranged barcode (BC) pairs to the total number of reads that contain parental BC pairs in technical replicates of each Cre transfection sample. FIG. 6B. Venn diagram depicting the overlapping relationships between Cre transfection samples for the subset of SVs that are detected in both technical replicates of each sample. FIG. 6C. Pie chart depicting the distribution of SV type for the rearrangements detected at 72 h. FIG. 6D. Scatter plot of rearrangement size (y-axis) vs. normalized read count (X-axis) for deletions and inversions detected at day 3. Pearson correlation is calculated between the log 10 values of the two metrics. FIG. 6E. Median size of inversions and deletions, weighted by their read count, for both the complete set of rearrangements (left) and those shared between technical replicates for a condition (right). FIG. 6F. Similar to the lower part of FIG. 5D, the bar plot shows the proportion of each SV type (from the complete set of rearrangements at 72 h) that is supported by at least one read in the IVT-seq data. FIG. 6G. Violin plots depicting the distribution of read counts for deletions, inversions, and translocations for the complete set of rearrangements detected at day 3. Inset within each violin plot is a box plot of the distribution with the median value depicted as a white line, the length of the box depicting the interquartile range, and the whiskers depicting the extent of the distribution. P-values are calculated using the non-parametric Mann-Whitney U test.

FIG. 7. Circos plots of the rearrangements detected at 72 h post-Cre transfection. Depicted rearrangements are from across the samples, including those that are not shared between technical replicates. Individual sectors of the Circos plots represent chromosomes arranged in ascending order (chr1-19, chrX, chrY) beginning at the 12 o'clock position and proceeding in a clockwise direction.

FIGS. 8A and 8B. Schematic of Bxb1 Genome-shuffle-seq and amplicon-seq library construction. Two Bxb1 shuffle cassettes bearing attB and attP sites respectively, are integrated into the genome in cis. Sites are flanked by unique barcodes (BC1, BC2), and 10× Genomics capture sequences (CS1, CS2) serve as binding sites for PCR primers for sequencing library construction. Recombination between the two sites results in the formation of novel attL and attR sites that are resistant to further recombination in the presence of Bxb1 alone. FIG. 8A. Recombination between two sites in the same orientation can result only in the formation of a genomic deletion+extrachromosomal circular DNA (ecDNA). FIG. 8B. Recombination between two sites in the opposite orientation can result only in the formation of a genomic inversion. Other annotations are the same as in FIG. 2A-2D. No Bxb1-mediated rearrangements lead to the formation of cassettes with the same primer binding site on both sides, eliminating the risk of suppressive PCR and ensuring the events are detectable in amplicon-seq.

FIGS. 9A-9C. Integration and mapping of attB/P and IoxP shuffle cassette libraries in mESCs and K562s. FIG. 9A. Schematic of experimental protocol for the generation of mESC and K562 populations with high MOI integration of shuffle cassettes. FIG. 9B. Number of insertion sites with unique barcodes (y-axis) across chromosomes of varying lengths (X-axis). The dotted line indicates a linear regression model fit, and the shaded gray areas indicate the 95% confidence interval. FIG. 9C. Left: pie chart depicting the distribution of assignments to BL6 or CAST alleles for attB/P shuffle cassettes whose genomic coordinates were mapped with high confidence in mESCs. Middle and right: pie charts depicting the distribution of attB and attP sites mapped at high confidence in mESCs and K562s, respectively.

FIGS. 10A-10H. Bxb1 recombinase mediates the induction of long-lived SVs in two mammalian cell types and reveals selection pressures. FIG. 10A. Experimental schematic. Libraries of shuffle cassettes bearing Bxb1 attB/P sites or Cre IoxP sites were integrated into human K562s or mouse ESCs. Rearrangements were induced by transient transfection of a recombinase-expressing plasmid, and cells were collected at the indicated time points for rearrangement detection by amplicon-seq. FIG. 10B. Number of novel (i.e., non-parental) barcode combinations at ≥2 UMI detected in each experimental condition by amplicon-seq. Colors indicate those rearranged barcode combinations found in one technical replicate or both. Left: K562s, bearing either IoxP or attB/P shuffle cassettes, exposed to either Cre or Bxb1 recombinase, from day 3 or 6. Right: mESCs, bearing attB/P shuffle cassettes, exposed to either Cre or Bxb1 recombinase, from day 3, 5, or 7. Note that technical replicates were performed for Bxb1-treated populations but not for Cre-treated populations. FIG. 10C. Number of novel barcode combinations at ≥2 UMI detected in each sample of the indicated K562 bottlenecked populations. A and B correspond to two independent bottlenecked populations that were sampled for Bxb1-treated attB/P+K562s per starting cell size, whereas only one bottlenecked population (A) was sampled for Cre-treated IoxPsym+K562s. Note that for each sample, data are from a single technical replicate from cells collected after expansion. FIG. 10D. Log-scale boxplots of inversion and deletion sizes, weighted by read count, at day 3 or 5 or post-bottlenecking/expansion. The horizontal solid line indicates the median, the length of the box depicts the interquartile range and the whiskers depict the extent of the distribution minus outliers. The underlying distribution is depicted by the overlaid points, with the size of each bubble reflecting the relative read count. Depicted p-values were calculated using a bootstrap analysis with 10,000 iterations, resampling the distribution with replacement. Left: K562s. Right: mESCs. FIG. 10E. Barplots depicting the proportion of total deletion and inversion reads at each time point that reflect a recombination between sites inferred to reside on different arms of the same chromosome. The number of events is indicated at the base of each bar. FIG. 10F. Log-scale violin plots depicting the distribution of read counts for the complete set of deletions, inversions, and translocations detected at the indicated time points. Inset within each violin plot is a box plot of the distribution with the median value depicted as a white line, the length of the box depicting the interquartile range, and the whiskers depicting the extent of the distribution. Depicted p-values were calculated using the non-parametric Mann-Whitney U test, comparing the read count distributions of translocations, deletions and inversions. Left: K562s. Right: mESCs. FIG. 10G. Schematic depicting the formation of balanced and unbalanced translocations. Unbalanced translocations can lead to the formation of acentric or dicentric chromosomes. FIG. 10H. Barplot depicting the proportion of total translocation reads at each indicated time point that are inferred to derive from balanced, acentric or dicentric translocations. The number of events of each type is indicated at the base of each bar.

FIGS. 11A-11D. Bxb1 Genome-shuffle-seq is specific and leads to more stable rearrangements than Cre in mESCs and K562s. FIG. 11A. Barplot depicting the number of reads for barcode combinations detected at ≥2 UMI that reflect an attL or attR site in Cre or Bxb1 transfected attB/P mESCs and K562s. The Bxb1 data in this figure reflect two technical replicates, and error bars represent 95% confidence intervals. The Cre data is from one technical replicate. FIGS. 11B-11D. Log 2 ratio of total reads with rearranged BC combinations to parental BC combinations in FIG. 11B mESC attB/P cells treated with Bxb1; FIG. 11C K562 attB/P cells treated with Bxb1 and K562 IoxP cells treated with Cre; FIG. 11D Bottlenecked and expanded populations derived from K562 IoxP cells treated with Cre and K562 attB/P cells treated with Bxb1. Data is from one technical replicate per bottlenecked population. The Bxb1 bars reflect amplicon-seq data from two independent bottlenecks, and error bars represent 95% confidence intervals.

FIG. 12A and FIG. 12B. Distributions of Bxb1-induced rearrangements in mESCs and K562s. FIG. 12A. Barplots depicting the proportion of deletions, inversions, and translocation reads in the total number of reads reflecting rearrangements in that sample. FIG. 12B. Barplots depicting the proportion of deletions, inversions, and translocation counts in the total number of rearrangements detected in that sample. The number at the bottom of each bar is the number of events of that rearrangement class detected at that time point.

FIGS. 13A-13D. Hundreds of ecDNAs are launched by Genome-shuffle-seq. t FIG. 13A. Stacked barplot depicting the proportion of rearranged barcodes called as deletions for which a matched barcode pair reflecting the other junction from the deletion event is detected within the same biological sample. In this analysis, technical amplicon-seq replicates coming from the same biological replicate were treated as one, and independent transfections or bottlenecking events were treated as separate biological replicates. The inset number reflects the number of events in each category. FIG. 13B. Barplot depicting the proportion of total read counts reflecting deletion events that come from the ecDNA vs. the genomic deletion scar in the samples at the indicated time points. The number at the base of the bar reflects the number of events in each category. FIG. 13C. Boxplot of the log 2 ratio of read counts for the barcode pairs that represent the ecDNA vs. genomic copy for the set of ecDNA-genomic deletion pairs that were both detected in the same sample. In this analysis, technical amplicon-seq replicates coming from the same biological replicate were treated as one, and independent transfections or bottlenecking events were treated as separate biological replicates. The number of such pairs is indicated underneath each sample. The horizontal solid line indicates the median, the length of the box depicts the interquartile range of the distribution, and the whiskers depict the rest of the distribution excluding outliers. The depicted p-value is calculated using the non-parametric Mann-Whitney U test. FIG. 13D. Boxplots of the size of ecDNAs and genomic deletion scars, weighted by their read count at the indicated time point. The horizontal solid line indicates the median, the length of the box depicts the interquartile range, and the whiskers depict the extent of the distribution minus outliers. The underlying distribution is depicted by the overlaid points, with the size of each bubble reflecting the relative read count. The depicted p-value is calculated using a bootstrap analysis with 10,000 iterations, resampling the distribution with replacement.

FIGS. 14A-14C. Single-cell detection of rearranged barcodes is specific to recombinase-treated cells. FIG. 14A. After fixation and IVT with T7 polymerase, cells contain both mRNA from endogenous genes as well as RNA from shuffle cassettes. Both of these RNA species can be captured using 10× Genomics gel beads that contain complementary sequences for the polyA on mRNA and capture sequences 1 and 2 (CS1, CS2) found on the T7-derived shuffle transcripts. FIG. 14B. Experimental schematic. At 72 h post-transfection, cells were sorted based on the activity of the Cre reporter, methanol-fixed, subjected to T7 IVT, and then sc-RNA-seq on the 10× Genomics platform. FIG. 14C. Number of novel barcode combinations detected either in the whole parental sample or a downsampled subset of the Cre sample in sc-RNA-seq.

FIGS. 15A-15H. Detection of SV identity and associated gene expression changes in single cells. FIG. 15A. Experimental schematic. The indicated populations were mixed prior to fixation and in situ transcription (IST) with T7 polymerase, after which cells were loaded onto two independent lanes of a 10× Genomics high-throughput (HT) chip. FIG. 15B. Barnyard plot of total T7 UMIs detected in mESCs or K562 cells at a threshold of ≥2 UMI per barcode pair. Each point represents a cell, colored by cell type assignment. The X-axis represents counts from shuffle-cassette barcodes originating from mESCs, and the y-axis represents counts from shuffle-cassette barcodes originating from K562s. FIG. 15C. In Lane 1, 11,252 cells were assigned to 138 independent clonotypes based on the complement of T7 barcodes detected within them. Here, these cells are visualized in UMAP space, colored by clone assignment. The plot on the right was generated following iterative dimensionality reduction on the indicated subset of cells from the global UMAP on the left. FIG. 15D. Barplot depicting the fraction of rearranged barcodes (BCs) detected in cells that are congruent with the clonotype assignment of that cell. Some barcodes were not assigned to a clone. FIG. 15E. In Lane 2, 14,727 cells were assigned to 53 clonotypes. Here, these cells are visualized in UMAP space and colored by clone assignment. The clone to which cells bearing the most frequent inferred rearrangement in this dataset, a 447 kb deletion on chromosome 15, is labeled. FIG. 15F. Map of fold-changes in gene expression vs. genomic coordinates for genes across chromosome 15. Genes with nominally significant (i.e. uncorrected) decreases in expression (one-sided Wilcoxon Rank Sum Test) and other genes are indicated by boxes. The y-axis depicts fold-change in expression between the cells in which the barcodes corresponding to the deletion were detected versus cells from the same clonotype in which parental barcode combinations were detected. Vertical region shaded indicates the span of the inferred 447 kb deletion. The black line indicates a moving average of fold-change with a window size of 3 genes. FIG. 15G. Quantile-quantile (Q-Q) plot of observed-log 10 p-values from a one-sided Wilcoxon Rank Sum Test of fold-changes against expected-log 10 p-values from a uniform distribution for genes across chromosome 15, in cells from a single clonotype inferred to either have or not have the 447 kb deletion. The red dotted line represents the expected relationship under the null hypothesis. Point size is proportional to the decrease in gene expression (1/fold-change) for that gene in cells with the rearrangement. Points are colored according to their proximity to the deletion. Names of genes encompassed by the deletion are colored red. FIG. 15H. Histogram of the mean fold-changes for rolling windows of 3 genes throughout the genome, comparing the same sets of cells, i.e., those from a single clonotype inferred to either have or not have the 447 kb deletion on chromosome 15. The 3-gene window fully overlapping with the deletion (USP8, TRPM7, SPPL2A) is indicated as “Deletion Window”, and windows with one or two of these genes are indicated as “Partial Overlap with Deletion Window”. The depicted p-value (uncorrected) is calculated from the z-score of the deletion window with the rest of the normal distribution.

FIGS. 16A-16H. High-quality transcriptomes are recovered along with T7-transcribed barcodes post methanol fixation. FIGS. 16A and 16B. Scatter plots of mitochondrial RNA fraction vs. transcriptome unique molecular identifier (UMI) counts per cell detected in Lane 1 and Lane 2. Red lines indicate thresholds that indicate the cells that were carried forward in the analysis. The cells within these thresholds that are associated with a rearranged barcode pair at ≥2 UMI are colored yellow. FIGS. 16C and 16D. Histogram of total T7-derived UMIs per cell barcode (BC) with the median value represented by a vertical dotted gray line in Lane 1 and Lane 2, respectively. FIGS. 16E and 16F. Histogram of total shuffle BC combinations detected per cell in T7 transcripts in Lane 1 and Lane 2, respectively. Median value is again depicted by a vertical dotted gray line. FIGS. 16G and 16H. Scatter plot of total transcriptome UMI counts (X-axis) versus total T7 UMI count per cell (y-axis) in Lane 1 and Lane 2 respectively.

FIGS. 17A-17C. Precision of T7 transcript detection is high at the ≥2 UMI threshold. FIG. 17A. Barnyard plots of total T7 UMIs detected in mESCs or K562 cells at various UMI thresholds per barcode pair. Each point represents a cell, colored by assignment to mESC or K562. The X-axis represents counts from shuffle-cassette barcodes originating from mESCs, and the y-axis represents counts from shuffle-cassette barcodes originating from K562s. Different panels correspond to different UMI thresholds for the detection of shuffle-cassette barcodes. FIG. 17B. Proportion of UMIs for species-specific barcodes from T7 transcripts detected in mouse or human cells at various UMI thresholds. The black line indicates the proportion of UMIs detected in cells of the indicated species at the respective UMI thresholds. FIG. 17C. Mean precision and recall of detected T7 barcodes in Lane 1 cells compared to the expected barcode list from respective clonotypes to which the cells are assigned (y-axis), at various UMI thresholds (X-axis). Only major clonotypes (n=123) with 10 cells or more assigned are included in the precision recall analysis.

FIGS. 18A-18F. Assignment of cells to clonotypes based on the complement of T7 barcodes detected.

FIG. 18A. In Lane 1, 11,252 cells were assigned to 138 independent clonotypes. Here, these cells are visualized in UMAP space, colored by clone assignment. The plot on the right was generated by iterative dimensional reduction on the indicated subset of cells from the global UMAP on the left. Cells are colored by the total number of T7-derived unique molecular identifiers (UMIs) detected in that cell. FIG. 18B. In Lane 2, 14,727 cells were assigned to 53 clonotypes. Other annotations are the same as in FIG. 18A). FIG. 18C. The cells assigned to clones in Lane 1, colored by the species of origin based on the transcriptome of each cell. Clones are largely composed of cells coming from a single species, as expected. FIG. 18D. Pie chart depicting the number of human (K562) and mouse (mESC) clones detected in single-cell data from Lane 1. FIGS. 18E and 18F. Barplot of the proportion of cells in each category that were assigned to a clone for Lane 1 (FIG. 18E) or Lane 2 (FIG. 18F). Clonotype assignment is determined by the set of T7 barcodes (BCs) within them, detected with ≥2 UMI. Cells were considered assigned to a clone if at least 75% of the T7 BCs detected in that cell at >2 UMI belong to that specific clone. The number of cells in each category is indicated by the number at the base of each bar.

FIGS. 19A-19F. Characteristics of rearranged T7-derived barcodes in single-cell data. FIG. 19A and FIG. 19B. Number of rearranged barcode (BC) combinations that are detected in Lane 1 (FIG. 19A) and Lane 2 (FIG. 19B), at successive stages of filtering: the cells, rearrangements detected at ≥2 UMI, rearrangements at ≥2 UMI in cells that could be assigned to a clonotype and lastly, rearrangements for which the identity of the rearranged BC pair was congruent with the clonotype assignment. That is, both BCs were detected in the same parental clone. FIG. 19C and FIG. 19D. Histogram depicting the number of unique cell barcodes associated with a particular rearrangement in Lane 1 (FIG. 19C) and Lane 2 (FIG. 19D). FIG. 19E and FIG. 19F. Histogram depicting the number of rearranged barcodes detected per cell BC in Lane 1 (FIG. 19E) and Lane 2 (FIG. 19F).

FIGS. 20A-20C. Distribution of rearrangements detected in single-cell data and comparison to bulk amplicon-seq. FIG. 20A. Barplot depicting the proportion of total rearranged barcodes detected across the cells, filtered as described in S18A for Lane 1 and Lane 2. The number of events in each category are indicated at the base of the bar. FIG. 20B. The proportion of the unique set of rearranged barcodes corresponding to inferred deletions, inversions and translocations in each sample and their detection status in matched, bulk amplicon-seq data is depicted as a barplot. FIG. 20C. Boxplot of the normalized read count from bulk amplicon-seq data for the set of rearranged barcodes found only in the bulk amplicon-seq data from the K562 1 k bottlenecks, or those detected in both the bulk and Lane 2 single-cell data. The p-value is calculated using the non-parametric Mann-Whitney U-test.

FIGS. 21A-21C. Genomic neighborhood of the chromosome 15 deletion and downsampling analysis. FIGS. 21A and 21B. Graphics from the UCSC Genome Browser of the indicated coordinates. K562 bulk RNA-seq data are from the ENCODE project, and gene-enhancer interactions are from the GeneHancer database (61,62). Genes with nominally statistically-significant decreases in gene expression in deletion-containing cells are labeled. In FIG. 21B, genes tested but insignificant are also labeled and in a box, and genes insufficiently expressed to qualify for being tested are labeled and double underlined. The gray shaded region indicates the extent of the deletion. FIG. 21C. Effects of sample size of cells with the rearrangement on p-value and estimated fold-change for the three genes in the deleted region. Shaded regions represent the 95th percentile interval of outcomes after 100 sampling trials. Horizontal dotted lines represent the significance level of p<0.05 and the expected fold-change of 0.66, respectively, without correction for multiple hypothesis testing.

FIGS. 22A and 22B. Conditional selectable markers increase the proportion of cells bearing SVs in Genome Shuffle-seq. FIG. 22A. Schematic depicting shuffle-cassette design with a split Blasticidin resistance marker (BsdR) across two constructs that is reconstituted upon Bxb1 recombination at attB/P sites. BC=barcode. FIG. 22B. Barplot depicting the fraction of recombined attL/attR sites detected in a pilot test of this strategy in HEK293T cells, at two time points, with and without Bxb1 or Blasticidin (BSD) treatment.

### DETAILED DESCRIPTION

The present disclosure provides shuffle cassettes and methods of using shuffle cassettes in “genome shuffle sequencing” (Genome Shuffle-seq). As used herein, “genome shuffle sequencing” or “Genome Shuffle-seq” refers to the methods, design, and methodology that enable the multiplex generation and mapping of thousands of genomic structural variants SVs (deletions, inversions, translocations, extrachromosomal circles) throughout mammalian genomes, resulting in a complex genetic rearrangement to cellular genotypes without the requirement for clonal isolation. In particular embodiments, a shuffle cassette includes a recombination site, flanked by a pair of barcodes, a pair of primer binding sites, and a pair of promoters. The shuffle cassettes are introduced into a host cell's genome. Rearrangement of these shuffle cassettes occurs after providing a recombinase targeting the recombination site of the shuffle cassettes. The rearrangement results in new and unique barcode combinations generated from the barcodes from shuffle cassettes introduced on the same chromosome in the same orientation, indicative of a deletion, or in opposite orientations, indicative of an inversion. The new barcode combinations can then be easily read out via sequencing to determine the identity of the structural variants. After integration of the shuffle cassettes into a host cell's genome, the shuffle cassette's location and orientation can be mapped by administering a polymerase and comparing the location and orientation of barcodes with the flanking sequences of the genome. In particular embodiments, the resulting genomic information of the cell can be determined.

Aspects of the current disclosure are now described with additional details and options as follows: (I) Overview of Shuffle Cassette and Genome Shuffle Sequencing; (II) Shuffle Cassette Architecture; (II)-A Recombination Sites and Recombinase; (II)-B Barcodes and Primer Binding Sites; (II)-C Promoters; (II)-D Payload; (III) Vectors and Delivery Systems; (IV) Exemplary Embodiments; (V) Experimental Examples; (VI) Selected References; (VII) Closing Paragraphs. These headings do not limit the interpretation of the disclosure and are provided for organizational purposes only.

### (I) OVERVIEW OF SHUFFLE CASSETTE AND GENOME SHUFFLE SEQUENCING

The present disclosure provides a high-throughput functional genomics platform, referred to herein as “Genome Shuffle-seq” (genome shuffle sequencing), which can be used for the multiplex generation, mapping, and/or functional characterization of thousands of genomic structural variants (SVs) and extrachromosomal DNAs (ecDNAs) within a heterogeneous cell population. Unlike traditional methods that rely on labor-intensive clonal isolation, genotyping, or expensive whole-genome sequencing (WGS), Genome Shuffle-seq directly links diverse genetic rearrangements to cellular phenotypes, such as transcriptomic signatures, at base-pair resolution. In particular embodiments, a shuffle cassette described herein includes a recombinase site flanked by a pair of unique barcodes, a pair of primer binding sites, and a pair of promoters.

The genome shuffle sequencing workflow is facilitated by the optimized architecture of the shuffle cassettes described herein.

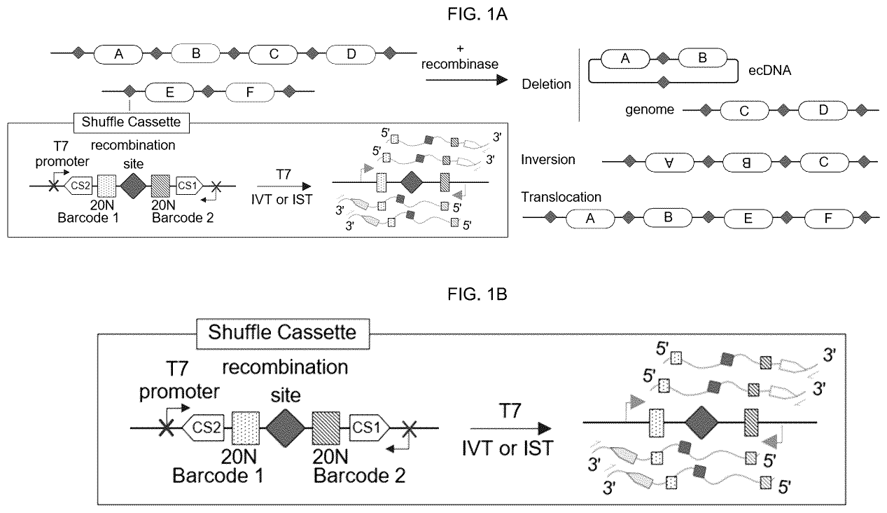

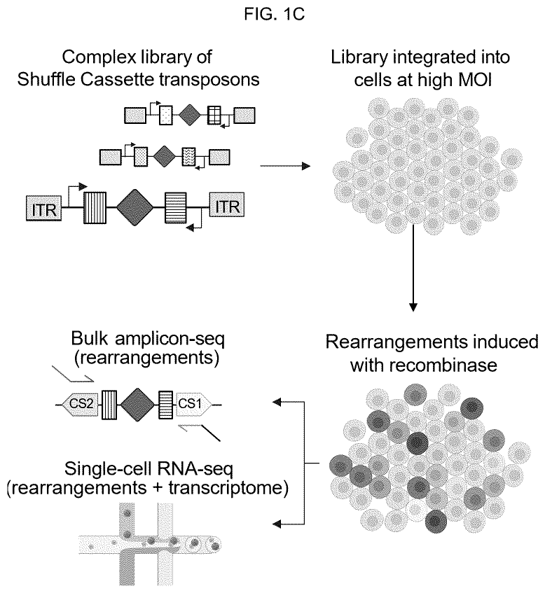

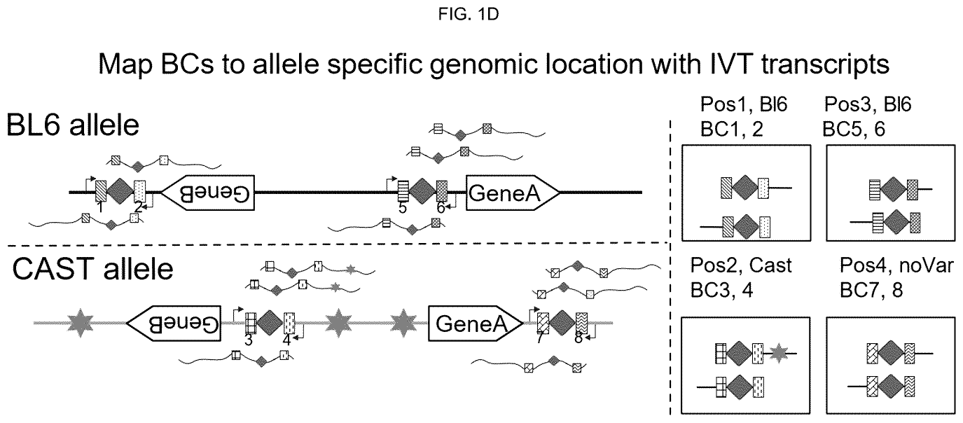

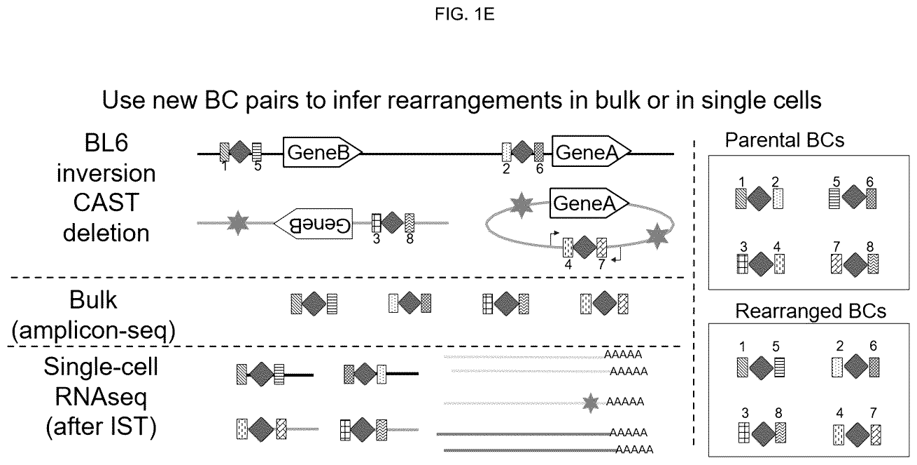

Integration of shuffle cassettes. As illustrated in FIGS. 1A-1E, shuffle cassettes are engineered to facilitate three functions: (1) the precise mapping of genomic integration coordinates via orthogonal phage promoters; (2) the stochastic generation of a diverse repertoire of SVs, including deletions, inversions, translocations, and ecDNAs, via site-specific recombinase (SSR); and (3) the efficient recovery of genotype-to-phenotype data through unique molecular barcoding of each cassette in a population of cassettes. In particular embodiments, the integration process initiates with the delivery of shuffle cassettes into a host cell's genome (e.g., mESCs or HEK293T cells). In particular embodiments, the introduction of the shuffle cassette into a host cell is achieved via a high multiplicity of infection (MOI) transposition system, such as a PiggyBac transposon vector.

Initial mapping. Following integration, the primary genomic coordinates and orientation of each cassette are identified. This is achieved by activating a phage promoter (e.g., SP6) within the cassette to drive in vitro transcription (IVT) or in situ transcription (IST) of the flanking genomic DNA. The resulting transcripts, which contain cassette-specific barcodes and adjacent genomic sequences, are sequenced to establish a high-resolution map of the “parental” genome state before any shuffling occurs.

Induced genomic rearrangement. Once the parental integrations are mapped, genomic rearrangement is triggered by the introduction or activation of an SSR. The recombinase acts upon the recombination sites (e.g., Bxb1 attB/attP or Cre IoxPsym) embedded within the shuffle cassettes. Because the cassettes are distributed throughout the genome, the recombination events stochastically generate thousands of unique structural variants, including deletions, inversions, and chromosomal translocations, as well as circularized ecDNA species. In particular embodiments, asymmetric sites (e.g., Bxb1) are used to ensure the stability of the resulting rearrangements and to avoid PCR suppression during downstream detection. The genomic rearrangements can occur between the shuffle cassettes after providing the SSR. This leads to new barcode combinations that reflect the nature of the rearrangement. These new barcode combinations are new and unique compared to the “parental” barcode combinations. In particular embodiments, the new barcode combinations include the barcodes from shuffle cassettes introduced on the same chromosome in the same orientation, indicative of a deletion, or in opposite orientations, indicative of an inversion.

High-resolution mapping and genotyping. The identity of the newly formed SVs and their functional consequences are determined. The identity of the SVs can be decoded from the new barcode combinations generated following the genomic rearrangement. Promoters, such as T7 or T3 promoters, within each integrated cassette drive the transcription of the rearranged genomic regions and thereby capture a fingerprint of the rearranged barcodes in RNA. In particular embodiments, the fingerprint can be read out in a single-cell RNA sequencing. Bulk analysis or single-cell analysis can be performed. For population-level insights, the resulting transcripts are analyzed via bulk amplicon sequencing to quantify the diversity of SV types. To link specific SVs to gene expression changes (phenotypes), individual cells are analyzed via scRNA-seq. In particular embodiments, the mapping includes associating each pair of barcodes within each of the shuffle cassettes with a genomic coordinate based on genomic sequences flanking each of the shuffle cassettes. The new and unique barcodes provided in each integrated cassette allow for the unambiguous assignment of a specific structural rearrangement to the transcriptomic state of an individual cell, enabling the functional characterization of SVs at an unprecedented scale.

### (II) SHUFFLE CASSETTE ARCHITECTURE

As illustrated in FIG. 1B, an example shuffle cassette includes from 5′ to 3′: a first promoter, a first primer binding site, a first barcode, a recombination site, a second barcode, the second primer binding site, and a second promoter. In another example, a shuffle cassette includes from 5′ to 3′: a first promoter, a first primer binding site, a first barcode, a recombination site, a second barcode, a second primer binding site, a second promoter, and a payload.

The first promoter (e.g., T7, T3, or SP6) is oriented to drive transcription into the flanking genomic DNA. The second promoter is oriented convergently or divergently relative to the first promoter, depending on the mapping strategy. The first primer binding site is a sequence for PCR amplification or sequencing library construction. The second primer binding site facilitates the capture of the reciprocal end of the cassette. The barcodes are unique molecular identifiers (e.g., a random 20-nucleotide sequence) that tag the specific integration event. The recombination site is a site-specific sequence recognized by a cognate recombinase.

### (II)-A Recombination Sites and Recombinase

In particular embodiments, the shuffle cassette includes at least one recombination site. The recombination site is selected based on the desired nature of the rearrangement (e.g., reversible vs. irreversible) and the target host cell environment. In particular embodiments, the recombination site includes a IoxPsym, IoxP, attB/attP, FRT, Rox, attL/attR, att, Gix, a synthetic recombination site, or an RNA-guided recombination site. Symmetric sites, such as IoxPsym, facilitate deletions, inversions, and translocations at comparable frequencies. Asymmetric/Heterotypic sites, such as attB and attP, recombine to form attL and attR site.

The present disclosure provides a recombinase that specifically targets and catalyzes the recombination of the sites provided on the shuffle cassettes. Suitable recombinase includes: Cre recombinase, Bxb1 integrase, FLP recombinase, λ phage integrase, PhiC31 integrase, Dre recombinase, TP901-1 integrase, or Gin recombinase.

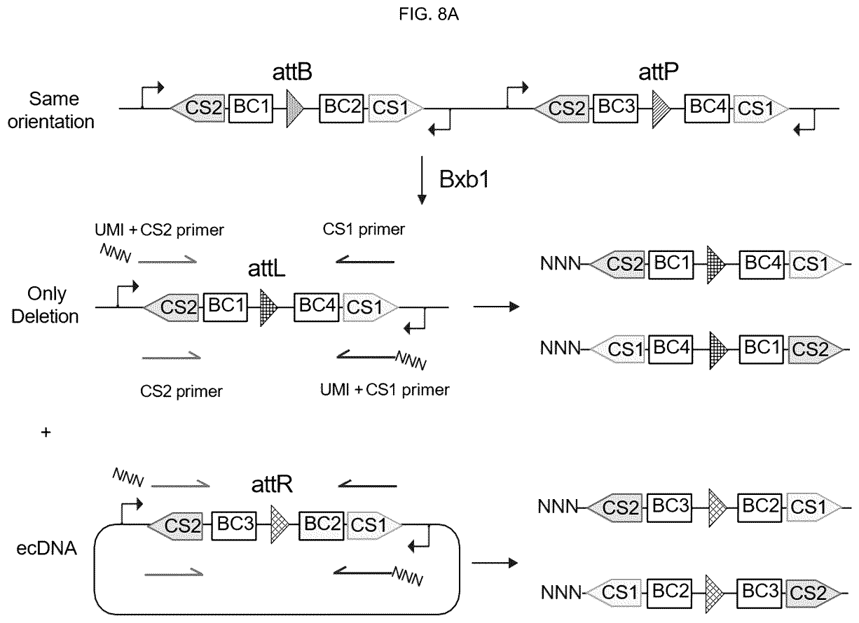

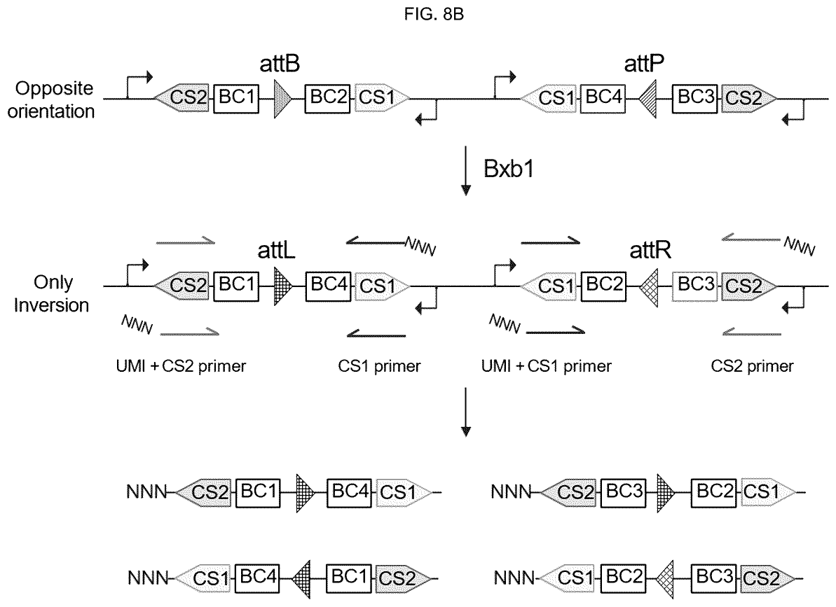

As illustrated in FIGS. 8A and 8B, the present disclosure utilizes Bxb1 integrase in combination with attB and attP recombination sites. In this example, at least two shuffle cassettes are integrated into the host genome in cis. These recombination sites are flanked by unique barcodes (BC1, BC2) and 10× Genomics capture sequences (CS1, CS2), which serve as specialized binding sites for PCR primers during the construction of sequencing libraries.

### (II)-B Barcodes and Primer Binding Sites

In particular embodiments, each shuffle cassette includes at least two barcodes. In particular embodiments, each shuffle cassette includes a first barcode and a second barcode, and the first barcode has a different sequence compared to the second barcode. These barcodes serve as unique molecular tags that identify the specific integration event of a cassette within the host genome. In particular embodiments, each barcode includes a random sequence of nucleotides of any reasonable length. In particular embodiments, each barcode includes 2-50 nucleotides in length or 15-25 nucleotides in length. In particular embodiments, each barcode includes 15, 16, 17, 18, 19, 20, 21, 22, 23, 24, or 25 nucleotides in length. Recombination shuffles these barcodes in cis, allowing specific SVs to be detected based on the appearance of novel barcode combinations that were not present in the parental cell population.

In particular embodiments, the shuffle cassette includes primer binding sites. These primer binding sites, also referred to as capture sequences (e.g., CS1 and CS2), facilitate the amplification of the cassette (e.g., via PCR) and its flanking genomic or transcriptomic DNA. In the example Bxb1-based system discussed above, the directional orientation of these primer binding sites prevents the formation of inhibitory “panhandle” structures during PCR, thereby eliminating the risk of suppressive PCR and ensuring that all rearrangement events remain detectable.

In particular embodiments, the primer binding sites include CS1 and CS2. In particular embodiments, CS1 includes the sequence GCTTTAAGGCCGGTCCTAGCAA (SEQ ID NO: 1). In particular embodiments, CS2 includes the sequence: GCTCACCTATTAGCGGCTAAGG (SEQ ID NO: 2).

### (II)-C Promoters

In particular embodiments, the shuffle cassette includes at least two promoters. These promoters are selected for their extreme specificity and high processivity, allowing for rapid and accurate transcription of genomic sequences flanking the cassette integration sites. They are also designed to be inert within mammalian cells (e.g., mESCs or K562s) under standard physiological conditions. They are activated specifically during in vitro transcription (IVT) or in situ transcription (IST) by the introduction of their respective cognate RNA polymerases after cell fixation.

In particular embodiments, the promoters are inducible promoters. In particular embodiments, the promoters are inert in a host cell. These promoters serve as molecular “on/off” switches that allow for precise temporal control over the expression of cassette components or payloads (e.g., the recombinase or a selectable marker). Suitable inducible promoters include tetracycline-responsive promoters (e.g., Tet-On/Off), which are activated by the presence of an inducer like Doxycycline, or chemically regulated variants such as CreERT2, which are activated by Tamoxifen.

In particular embodiments, the promoters are phage promoters. In particular embodiments, the promoters include a T7 promoter, a T3 promoter, or an SP6 promoter. In particular embodiments, the T7 promoter includes a sequence as set forth in TAATACGACTCACTATAG (SEQ ID NO: 3).

### (II)-D Payload

The shuffle cassette may further include a payload design for the identification enrichment of cells containing successful SVs. In particular embodiments, the payload includes at least one of a selectable marker, a detectable marker, or a splitable marker. Cells transduced with the shuffle cassette, including a payload, can be identified by selection or detection. Examples of payloads include genes providing resistance to antibiotics (e.g., Blasticidin resistance, BsdR) to filter out non-rearranged cells, fluorescent proteins (e.g., GFP, RFP) used for visual tracking or FACS-based sorting of cells undergoing “shuffling” events; a splitable (or split-selectable) marker system where a functional marker is reconstituted only after a specific genomic rearrangement (84).

The current disclosure provides a method of introducing at least two different shuffle cassettes into a host cell using a splitable marker. In particular embodiments, each of the shuffle cassettes includes a recombination site, a pair of barcodes flanking the recombination site, at least two promoters, at least one primer binding site, and a half of a splitable marker. The recombination site, barcodes, and promoters are embedded within a synthetic intron. The method described herein then provides a recombinase targeting the recombination site of each of the shuffle cassettes to the host cell and reconstitutes the two shuffle cassettes into a recombinant construct, thereby inducing recombination of the recombinant construct into the host cell's genome.

In particular embodiments, the splitable marker includes a split Blasticidin resistance (BsdR) marker, a split green fluorescent protein (GFP) marker, or a split red fluorescent protein (RFP) marker. In particular embodiments, wherein the at least two promoters include phosphoglycerate kinase (PGK) promoter or a tetracycline-responsive promoter (TRE).

### (III) VECTORS AND DELIVERY SYSTEMS

The present disclosure provides various vehicles and methods for introducing the shuffle cassettes described herein into a host cell. In particular embodiments, the shuffle cassette is provided to a cell by a vector. As used herein, the term “vector” refers to a nucleic acid molecule capable of transporting another nucleic acid to which it has been linked. The vectors of the present disclosure are engineered to ensure the stable or transient presence of the shuffle cassette within the host cell and the integration of the cassette into the host cell genome.

The host cell may be any eukaryotic cell capable of supporting site-specific recombination and intronic splicing. Exemplary host cells include, but are not limited to, mammalian cells (e.g., human, mouse, rat, non-human primate), plant cells (e.g., Arabidopsis thaliana, rice (Oryza sativa), maize (Zea mays), or tobacco (Nicotiana tabacum), avian cells (e.g., chicken), fish cells (e.g., zebrafish), amphibian cells (e.g., Xenopus), reptilian cells, insect cells (e.g., Drosophila, Spodoptera frugiperda), fungal cells (e.g., yeast, such as Saccharomyces cerevisiae, Schizosaccharomyces pombe), protozoan cells (e.g., Plasmodium, Trypanosoma), and bacterial cells (e.g., E. coli, Bacillus subtilis).

The selection of a delivery vector may be optimized for the specific host cell type. For example, while PiggyBac and Sleeping Beauty systems are demonstrated in mammalian contexts, Tol2 and Mariner transposons are well-characterized for high-efficiency integration in fish and insect models.

In particular embodiments, the vector includes a transposon vector. Transposon vectors allow for the efficient, non-viral integration of the shuffle cassette into the host genome, often at multiple locations, which facilitates high multiplicity of infection (MOI) required for large-scale genome shuffling. In particular embodiments, the vector is a PiggyBac transposon vector. A PiggyBac transposon system typically utilizes a transposase enzyme (e.g., a hyperactive transposase such as hyPBase) that recognizes inverted terminal repeats (ITRs) flanking the shuffle cassette and catalyzes its integration into TTAA genomic sites. In particular embodiments, the vector is a Sleeping Beauty transposon vector, a Tol2 transposon vector, a Mariner transposon vector, a Frog Prince transposon vector, a TcBuster transposon vector, or a SpinON transposon vector.

In particular embodiments, the vector is a viral vector. Viral vectors are particularly useful for delivering shuffle cassettes to primary cells or difficult-to-transfect cell lines. In particular embodiments, the viral vector may include a retroviral vector or a lentiviral vector, which can provide stable, long-term expression by integrating the shuffle cassette into the host genome. Further embodiments may utilize adenoviral vectors or adeno-associated viral (AAV) vectors. These vectors may be configured for either integrative or episomal delivery depending on the required experimental duration and the nature of the structural variant (e.g., ecDNA) being studied.

In particular embodiments, the shuffle cassette is delivered via a targeted gene editing vector. The vector may be configured for site-specific integration using CRISPR/Cas9, zinc finger nucleases (ZFNs), or transcription activator-like effector nucleases (TALENs). This allows for the precise placement of shuffle cassettes at defined genomic loci to study the effects of rearrangements at specific regulatory regions.

The aforementioned vectors may be introduced into the host cell via a variety of standard delivery methods, including but not limited to lipofection (e.g., using Lipofectamine 3000), nucleofection (e.g., using Amaxa 4D systems), or electroporation. The choice of method is typically optimized based on the host cell type, such as mESCs grown on gelatin-coated plates or K562 cells grown in suspension.

The present disclosure also provides a method including introducing the shuffle cassette into a host cell's genome; and providing a recombinase to the host cell, thereby driving recombination of shuffle cassettes that are integrated in the host cell's genome. This method further includes mapping positions and/or orientations of the shuffle cassettes within the host cell's genome; and/or genotyping the host cell's genome after recombination. In particular embodiments, the host cell is a mammalian cell (e.g., an embryonic stem cell). In particular embodiments, the host cell is a mouse embryonic stem cell (mESC).

In particular embodiments, the shuffle cassettes disclosed herein enable the capture of genotype information in RNA. In particular embodiments, the genotype information can be read out using single-cell RNA sequencing, bulk amplicon sequencing, in vitro transcription sequencing (IVT-seq), or in situ transcription sequencing (IST-seq).

### (IV) EXEMPLARY EMBODIMENTS

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.
1. A shuffle cassette including a recombination site, a pair of barcodes including a first barcode and a second barcode flanking the recombination site, a first primer binding site, a second primer binding site, a first promoter, and a second promoter, wherein the first barcode has a different sequence compared to the second barcode.
2. The shuffle cassette of embodiment 1, wherein the recombination site includes IoxPsym, IoxP, attB/attP, FRT, Rox, attL/attR, att, Gix, a synthetic recombination site, or an RNA recombination site.
3. The shuffle cassette of embodiment 1 or embodiment 2, wherein the first barcode or the second barcode includes 2 to 50 nucleotides in length.
4. The shuffle cassette of any of the embodiments 1-3, wherein the first primer binding site or the second primer binding site includes the sequence as set forth in SEQ ID NO: 1 or SEQ ID NO: 2.
5. The shuffle cassette of any of the embodiments 1-4, wherein the first promoter or the second promoter includes an inducible promoter that is inert in a host cell.
6. The shuffle cassette of any of the embodiments 1-5, wherein the first promoter or the second promoter includes a T7 promoter, a T3 promoter, or an SP6 promoter.
7. The shuffle cassette of any of the embodiments 1-6, further including a payload including a selectable marker, a detectable marker, or a splitable marker.
8. A vector including the shuffle cassette of embodiment 1.
9. The vector of embodiment 8, wherein the vector is a transposon vector, a viral vector, or a targeted gene editing vector.
10. The vector of embodiment 9, wherein the transposon vector includes a PiggyBac transposon vector, a Sleeping Beauty transposon vector, Tol2 transposon vector, Mariner transposon vector, Frog Prince transposon vector, TcBuster transposon vector, or SpinON transposon vector.
11. A shuffle cassette library including a plurality of the shuffle cassettes of embodiment 1, wherein each of the shuffle cassettes includes a different first barcode and each of the shuffle cassettes includes a different second barcode, and wherein the first barcode of each of the shuffle cassettes is different from the second barcode within the same shuffle cassette.
12. A cell library including the shuffle cassette library of embodiment 11.
13. A method including introducing a plurality of the shuffle cassettes of embodiment 1 into a genome of a host cell; and providing to the host cell a recombinase targeting the recombination site of the shuffle cassettes, thereby inducing recombination between the shuffle cassettes introduced in the genome of the host cell to generate a structural variant and a set of barcode combinations, the set of barcode combinations including the barcodes from shuffle cassettes introduced on a same chromosome in a same orientation indicative of a deletion or in opposite orientations indicative of an inversion.
14. The method of embodiment 13, wherein the introducing includes transposition or retrotransposition.
15. The method of embodiment 13 or embodiment 14, wherein the providing the recombinase includes administering a plasmid encoding the recombinase.
16. The method of any of the embodiments 13-15, further including mapping positions and/or orientations of the shuffle cassettes within the host cell's genome before the providing.
17. The method of embodiment 16, wherein the mapping includes providing a polymerase that binds the first and/or second promoter of each of the shuffle cassettes; transcribing, using the polymerase, a transcript including shuffle cassette-specific barcodes and genomic sequences flanking each of the shuffle cassettes; sequencing the transcript; and associating each pair of barcodes within each of the shuffle cassettes with a genomic coordinate based on the genomic sequences flanking each of the shuffle cassettes.
18. The method of any of the embodiments 13-17, further including determining an identity of the structural variant by decoding the set of barcode combinations; and sequencing the structural variant, wherein the structural variant includes deletions, inversions, chromosomal translocations, or extrachromosomal circular DNA.
19. The method of any of the embodiments 13-18, further including genotyping the host cell after the providing the recombinase, the genotyping including single-cell RNA sequencing (scRNAseq), bulk amplicon sequencing, in vitro transcription sequencing (IVT-seq), in situ transcription sequencing (IST-seq), or performing polymerase chain reaction (PCR).
20. The method of any of the embodiments 13-18, further including in situ transcription sequencing followed by single-cell RNA sequencing after the providing the recombinase, thereby capturing an identity of the structural variant and transcriptomic data from the host cell, wherein the structural variant includes deletions, inversions, chromosomal translocations, or extrachromosomal circular DNA.
21. The method of any of the embodiments 13-20, wherein the host cell includes a mammalian cell, a plant cell, an insect cell, a bacterial cell, a fungal cell, a protozoan cell, a fish cell, an amphibian cell, a reptilian cell, or an avian cell.
22. A method including introducing at least two different shuffle cassettes into a genome of a host cell, each of the shuffle cassettes including a recombination site, a pair of barcodes flanking the recombination site, at least two promoters, at least one primer binding site, and a half of a splitable marker, wherein the recombination site, the barcodes, and the promoters are embedded within a synthetic intron; providing a recombinase targeting the recombination site of each of shuffle cassettes to the host cell; and reconstituting the at least two shuffle cassettes into a recombinant construct, thereby inducing recombination of the recombinant construct introduced in the genome of the host cell to generate a structural variant.
23. The method of embodiment 22, wherein the splitable marker includes a split Blasticidin resistance (BsdR) marker, a split green fluorescent protein (GFP) marker, or a split red fluorescent protein (RFP) marker.
24. The method of embodiment 22, wherein the at least two promoters comprise a phosphoglycerate kinase (PGK) promoter or a tetracycline-responsive promoter (TRE).
25. A kit including the shuffle cassette of embodiment 1,
26. The kit of embodiment 25, further including a recombinase including Cre recombinase, Bxb1 integrase, FLP recombinase, λ phage integrase, PhiC31 integrase, Dre recombinase, TP901-1 integrase, or Gin recombinase.
27. The kit of embodiment 25 or embodiment 26, further including a host cell.
28. The kit of any of the embodiments 25-27, further including sequencing components.
29. The kit of embodiment 28, wherein the sequencing components include a polymerase.

### (V) EXPERIMENTAL EXAMPLES

### Experimental Example 1. Multiplex Generation and Single-Cell Analysis of Structural Variants in Mammalian Genomes

Abstract. Studying the functional consequences of structural variants (SVs) in mammalian genomes is challenging because: 1) SVs arise much less commonly than single-nucleotide variants or small indels; and 2) methods to generate, map, and characterize SVs in model systems are underdeveloped. To address these challenges, Genome-Shuffle-seq was developed, a method that enables the multiplex generation and mapping of thousands of SVs (deletions, inversions, translocations, extrachromosomal circles) throughout mammalian genomes. The co-capture of SV identity with single-cell transcriptomes was also demonstrated, facilitating the measurement of SVs' impact on gene expression. Genome-Shuffle-seq will be broadly useful for the systematic exploration of the functional consequences of SVs on gene expression, chromatin landscape, and 3D nuclear architecture, while also initiating a path towards a minimal mammalian genome. At least some of the work described herein was published in Pinglay et al., Multiplex generation and single-cell analysis of structural variants in mammalian genomes. Science, 387 (6733), 2025 (doi: 10.1126/science.ado5978).

Introduction. Major classes of human genetic variation include SNVs, indels, simple sequence repeat variants, and genomic SVs (e.g., deletions, insertions, inversions, and duplications over 50 bp, and chromosomal translocations) (1,2). Historically, genomic SVs have been at a distinct disadvantage in terms of the ability to study their functional consequences, as compared to SNVs or indels. This is unfortunately the case for not only paradigms of genetic analysis that rely on living humans, but also genetic analyses conducted in in vitro or in vivo model systems.

For human genetics, de novo SVs are over 100-fold less frequent than de novo SNVs per human generation (3). Therefore, SVs are less likely to recur, and when they do recur, are less likely to do so in a way that permits the unambiguous implication of a specific functional unit (e.g. recurrent disruption of identical sets of genes or regulatory elements). This contrasts with the recurrence of disruptive SNVs or indels within a single gene or regulatory element, which allows for the assignment of causality in Mendelian studies. Furthermore, SVs' lower rate of de novo occurrence, together with a greater likelihood of deleterious fitness effects (because SVs disrupt orders-of-magnitude more base-pairs [bp] than SNVs or indels), contribute to an even greater numerical paucity among standing genetic variants in human populations (3-7). Far fewer SVs than SNVs or indels reach the common allele frequencies that would allow for the well-powered detection of phenotypic effects by genome-wide association studies (GWAS). To an extent, these limitations can be addressed with larger cohort sizes, but this has its limits. For example, although every possible SNV compatible with life is likely present in a living human (8), this is almost certainly not the case for all possible SVs.

For laboratory-based genetics, a plethora of strategies have been developed to introduce SNVs or indels into model systems for functional analysis. These include classic chemical mutagenesis screens as well as their modern equivalent, base editing screens (9). They also include using massively parallel DNA synthesis or mutagenic PCR to achieve saturation mutagenesis of a sequence of interest, which can then be studied either within or outside of its native genomic context (10, 11). Although many specific SNVs or indels generated by such methods have yet to be observed in a living human, their analysis can nonetheless be informative in myriad ways, e.g. for implicating specific genes in specific phenotypes (12), for systematically characterizing the distribution of effect sizes of regulatory or coding variants (10, 11,13), for pre-computing the clinical consequences of potential variants in disease-associated genes (11), for optimizing immunotherapies (14), etc. However, once again, SVs are at a clear disadvantage compared to SNVs or indels, here due to the relative immaturity of methods to generate and map SVs in model systems.

Consequent to these disadvantages, there remain numerous unanswered “structure-function” questions about the human genome that relate to its properties at the scale of SVs rather than SNVs or indels. Genes, exons, and cis-regulatory elements are scattered over vast distances, ordered and oriented in specific ways in specific genomes. However, the understanding of the functional implications of these distances, orders, and orientations arguably remains very shallow. For example, roughly one-quarter of the human genome is composed of gene deserts (15). Although patterns of conservation suggest at least some sequences within such deserts are functional, the deletion of even megabase-sized gene deserts yields viable mice with no discernible phenotype (16,17). Other non-genic SVs clearly cause Mendelian disorders, contribute to complex disease risk, or underlie evolutionary adaptations (18), but there are very few cases in which the mechanism is understood. Some mammalian genomes differ by over 1 billion bp in size from the human genome (19), and even between similarly sized mammalian genomes, such as mouse and human, over 1 billion bp have both been gained and lost over evolutionary time via structural variation (20). Beyond the germline, somatic SVs of various kinds, including some cancer-specific forms of structural variation like chromothripsis and ecDNAs (21,22), are increasingly recognized as playing roles in the initiation and progression of many human cancers.

Various strategies have been developed for engineering SVs. For example, site-specific recombinase (SSR) recognition sites can be introduced to specific locations, such that their recombination results in a specific SV, or even ecDNA species, of interest (23-25). However, this is labor-intensive, and only results in one or a handful of SVs to study. Alternatively, CRISPR/Cas9 can be used to simultaneously drive double-stranded breaks (DSB) at multiple locations, which can result in the generation of SVs, potentially even genome-wide (26-28). But CRISPR/Cas9-based SV induction is challenged by inefficiency, imprecision, DSB toxicity, and the absence of a means to efficiently map which cells harbor which (if any) induced SVs. Towards a genome-wide SSR-based approach, inspiring work by Sauvageau and colleagues deployed retroviral introduction of SSR recognition sites to generate a panel of mouse embryonic stem cells (mESC) clones bearing nested deletions covering 25% of the mouse genome (29,30). However, the method is still limited by the lack of an efficient means of mapping the initial SSR recognition site positions nor for genotyping post-induction SVs. In yeast, chromosome-specific or genome-wide “scrambles” were achieved by first building synthetic chromosomes containing many SSR recognition sites (31-33), but for mammalian genomes, whole chromosome or whole genome synthesis remains impractical. Finally, in the cases (including yeast) where a larger number of SVs are (potentially) induced, the recovery, verification, and/or quantitation of SVs relies on inefficient or expensive methods (e.g., single cell cloning, whole genome sequencing, karyotyping), which markedly limit what can be achieved, particularly for mammalian models.

Motivated by both the knowledge gaps and technical gaps surrounding SVs, Genome-Shuffle-seq was developed, a straightforward method for multiplex generation of large-scale SVs throughout a mammalian genome (FIGS. 1A-1E). An attribute of Genome-Shuffle-seq is that it facilitates the facile mapping and genotyping of the breakpoints of induced SVs at base-pair resolution within a population of cells. As a proof-of-concept, Genome-Shuffle-seq was applied to induce thousands of genomic SVs of several major forms (deletions, inversions, chromosomal translocations, ecDNAs) in two mammalian cell lines, and to map their coordinates at base-pair resolution without any need for whole genome sequencing. It is further demonstrated that the identities of induced SVs ca be co-captured as part of a single cell transcriptome, laying the foundation for pooled cellular screens of thousands to millions of mammalian SVs.

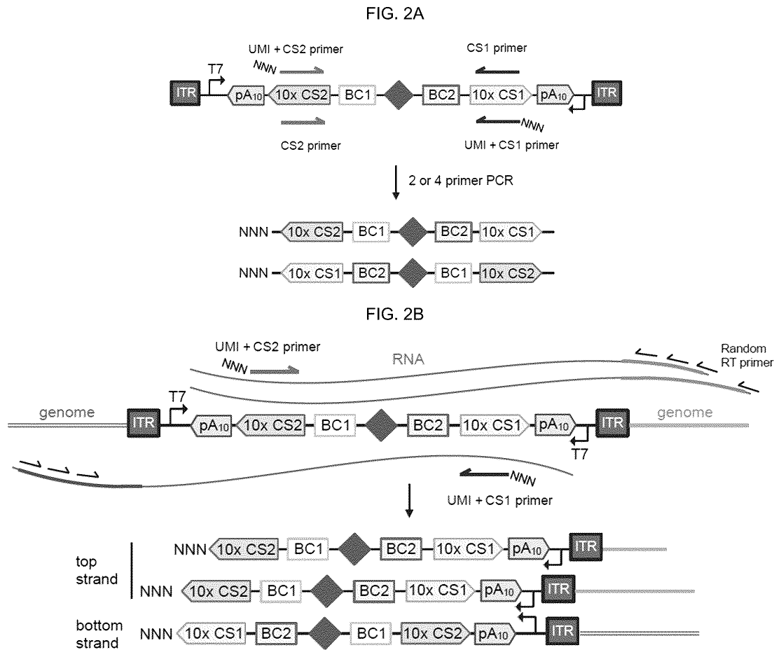

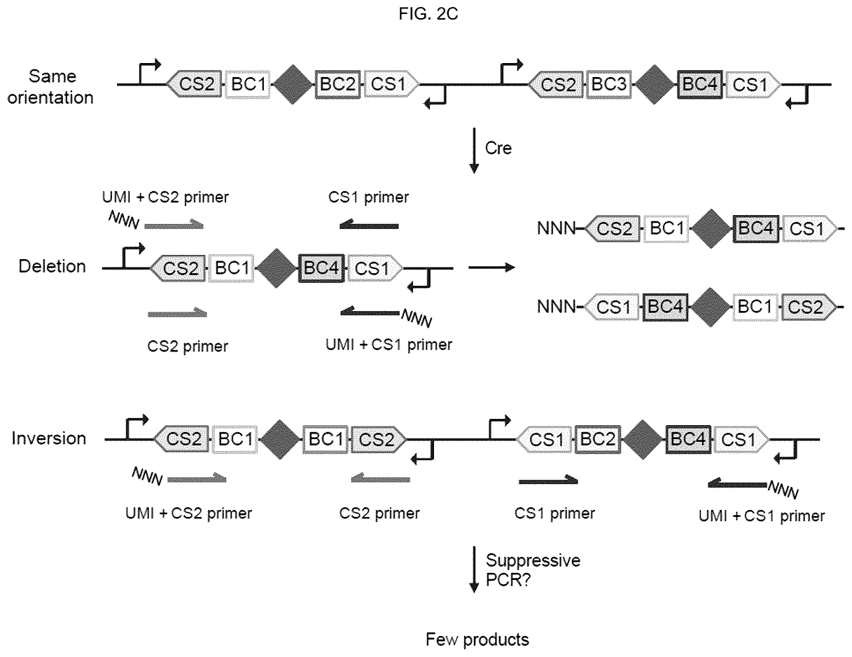

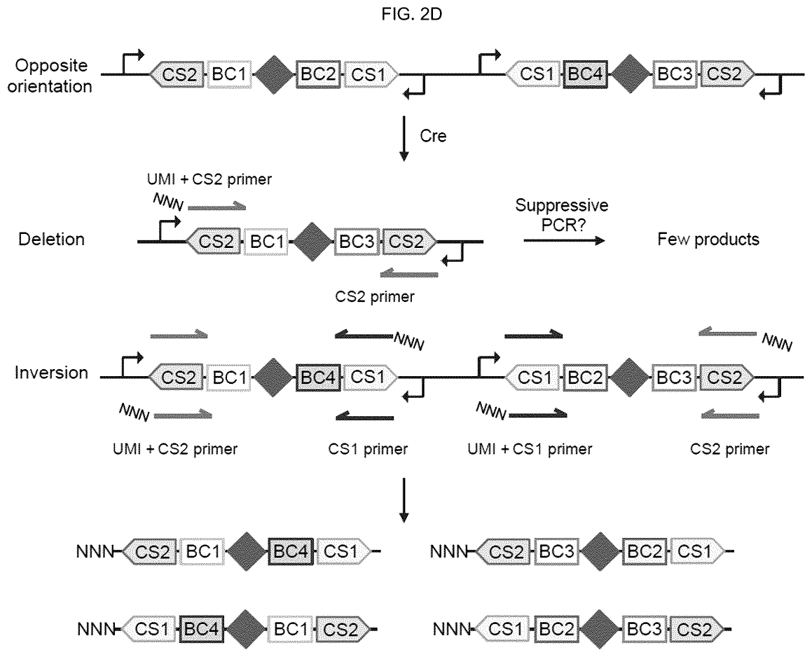

Design of Genome-Shuffle-seq. Genome-Shuffle-seq is based on the integration of “shuffle cassettes” into a mammalian genome (FIGS. 1A-1C). The shuffle cassettes are designed to facilitate the mapping of genomic integration coordinates, the generation of SVs via SSR between pairs of shuffle cassettes, and the efficient recovery of genotype information. The initial design of the shuffle cassette is 176 bp in length and has four features (FIG. 1B): 1) A IoxPsym site capable of being recombined with other IoxPsym sites at high efficiency by Cre recombinase. Recombination between two copies of this symmetric variant of the canonical IoxP site is expected to lead to both deletions and inversions at roughly equal frequencies (31,34), as well as translocations; 2) Immediately flanking the IoxPsym site, a pair of random 20 nucleotide (nt) barcodes, which uniquely tag each integration of the shuffle cassette, or its recombined derivatives; 3) Further flanking those, a pair of PCR primer binding sites (35) (FIG. 2A); and 4) At the outer boundaries, a pair of convergently oriented phage T7 RNA polymerase promoters which are inert in mammalian cells but can be activated after fixation for in vitro transcription (IVT) on genomic DNA or in situ transcription (IST) on fixed cells, with T7 polymerase (36,37) (FIG. 2B).

Once shuffle cassettes are introduced into the genome (e.g. randomly via transposition or retrotransposition at a high multiplicity of infection (MOI) or, alternatively, in a targeted fashion), their positions can be efficiently mapped by sequencing T7 IVT-derived transcripts from genomic DNA that contain both cassette-specific barcodes and genomic sequence flanking each integration, with a straightforward protocol recently described (37) (FIGS. 1D and 2B). Starting from a parental cell population in which each cell contains a distinct repertoire of integrated shuffle cassettes with mapped genomic locations, Cre recombinase is expected to induce SVs by driving recombination between shuffle cassettes integrated throughout the genome of the same cell. Because these recombination events shuffle which 20 nt barcodes are located in cis, specific SVs can be detected and quantified based on novel barcode combinations observed only in “post-shuffle” cells. A point worth noting is that both parental and novel combinations are detectable by sequencing PCR amplicons derived from the shuffle cassettes (FIGS. 1E, 2A). To genotype SVs alongside the capture of single cell transcriptomes, T7 IST can be performed after fixation but prior to scRNA-seq (36,37), creating an RNA fingerprint of which barcode combinations (and therefore which SVs) are present in association with each single cell transcriptome (FIG. 1E). Altogether, this strategy is designed to enable: 1) the multiplex generation of SVs in a population of mammalian cells; 2) the straightforward mapping of the breakpoints and nature of induced SV with no need for whole genome sequencing or karyotyping; and 3) the efficient genotyping and quantitation of SVs, either in bulk (from total DNA or RNA) or at single cell resolution (in conjunction with scRNA-seq).

Genome-Shuffle-seq enables the multiplex generation and haplotype-resolved mapping of thousands of SVs in BL6xCAST mouse ESCs. As a proof-of-concept, a complex library of shuffle cassettes was cloned into a PiggyBac transposon vector (37). This library was then randomly integrated into the genome of an F1 hybrid C57BL6/6JxCAST/EiJ (BL6xCAST) male diploid mESC cell line (FIG. 1C) (38). This cell line was chosen for three reasons. First, a heterozygous variant (SNV or indel) is present, on average, every 150 bp, which should facilitate the assignment of shuffle cassette integrations, as well as induced SVs, to one haplotype or the other (39). Second, it was reasoned that inducing large rearrangements in diploid cells would be less likely to cause cell death, relative to haploid cells. Finally, this mESC line can potentially be differentiated into diverse cell types or organoids, which might eventually facilitate the study of cell type-specific effects of SVs starting from one engineered cell population.

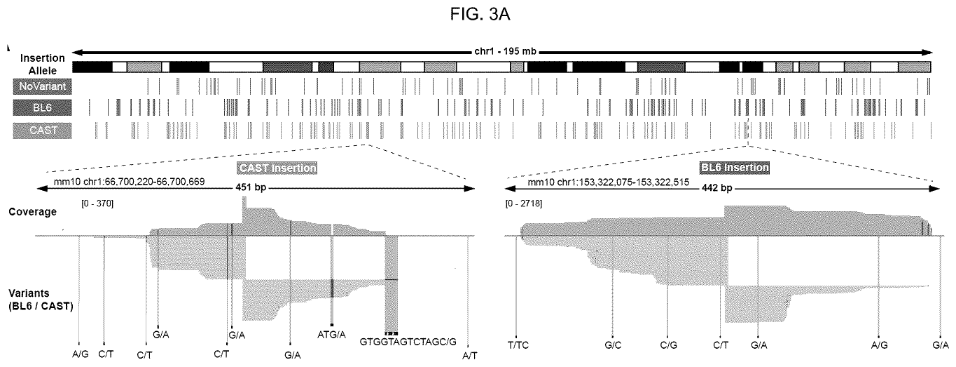

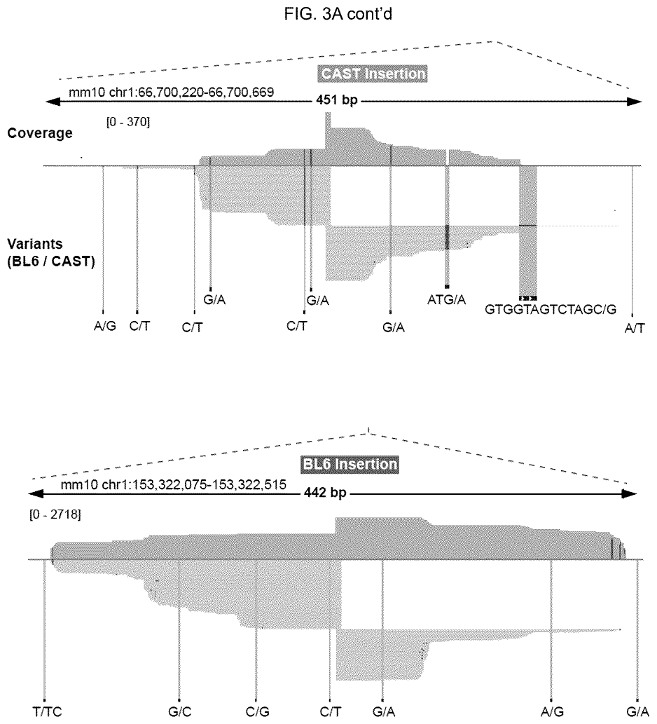

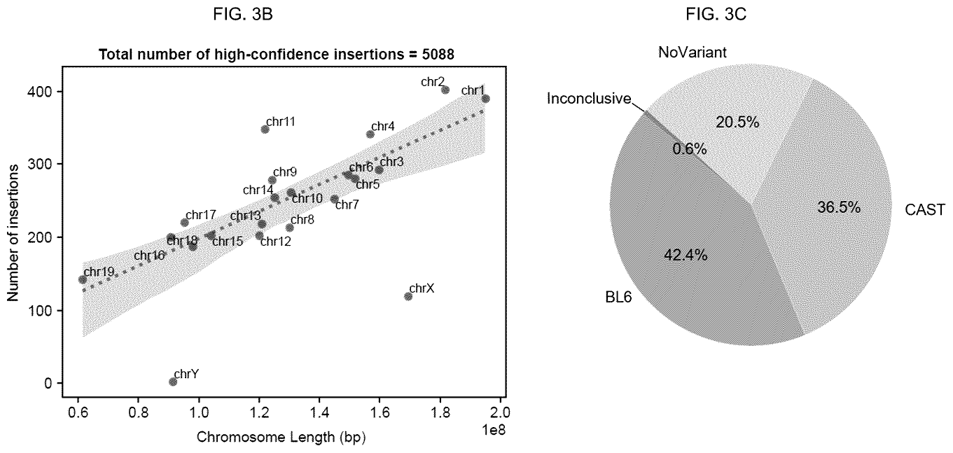

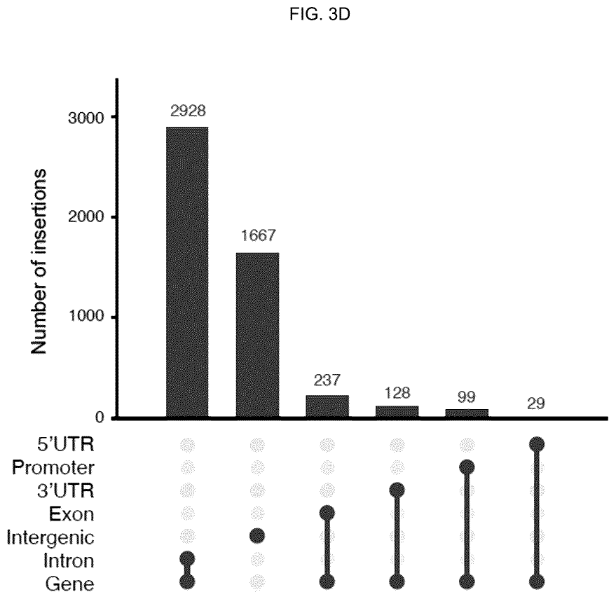

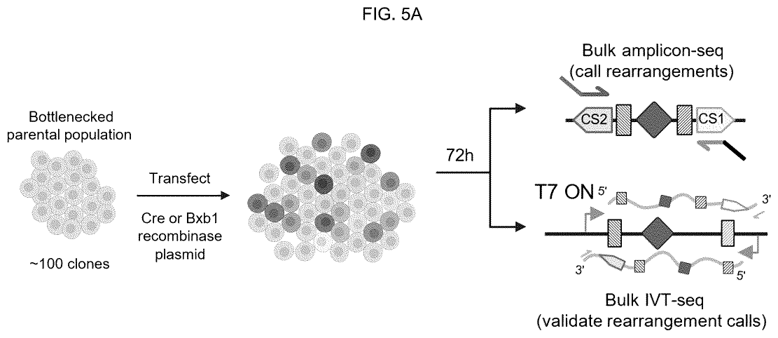

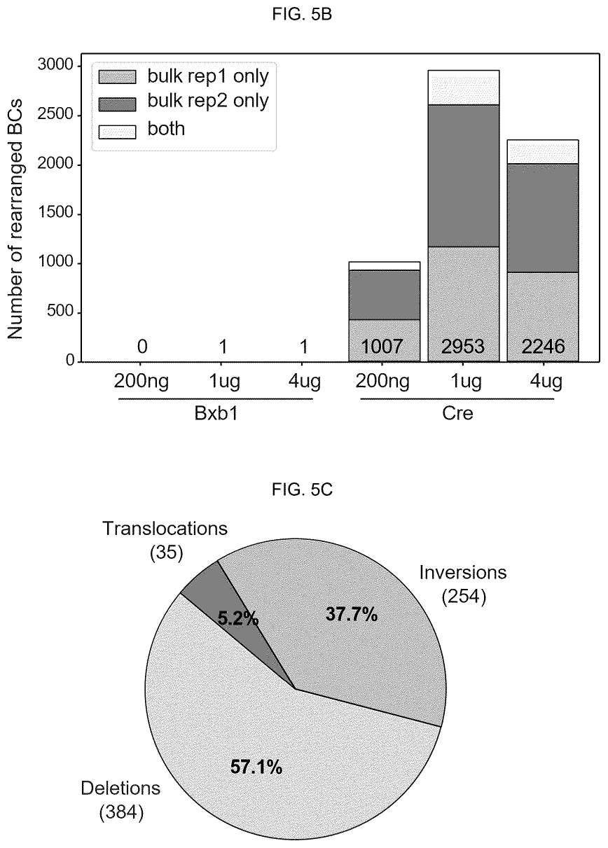

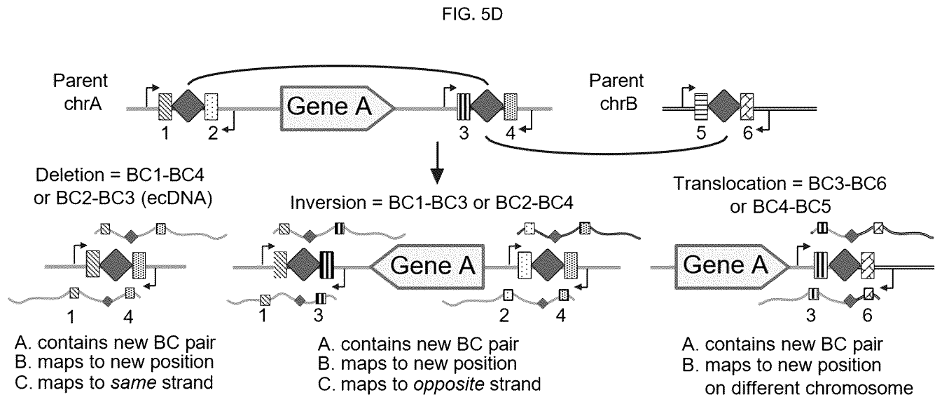

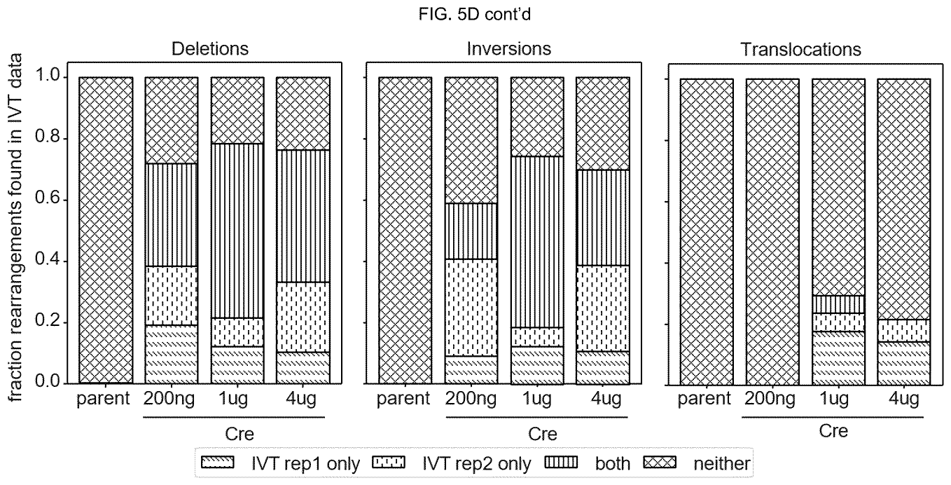

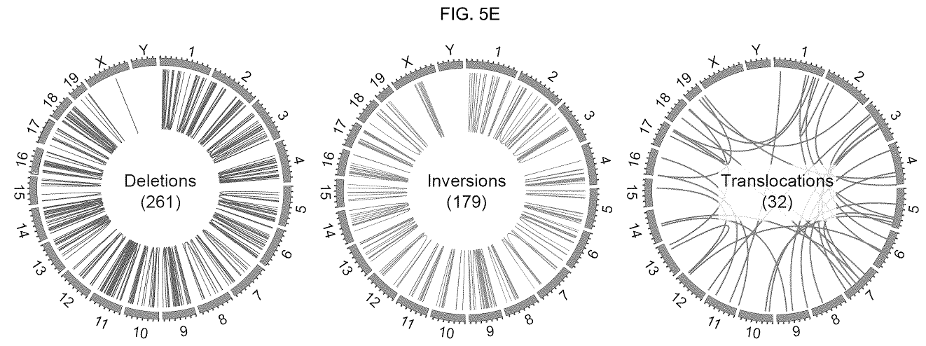

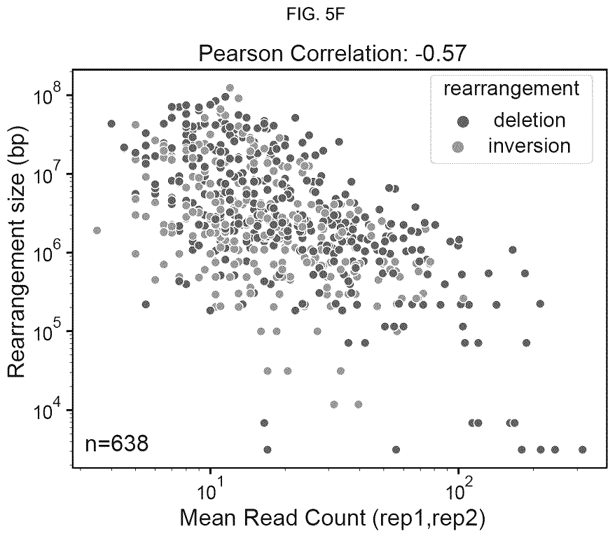

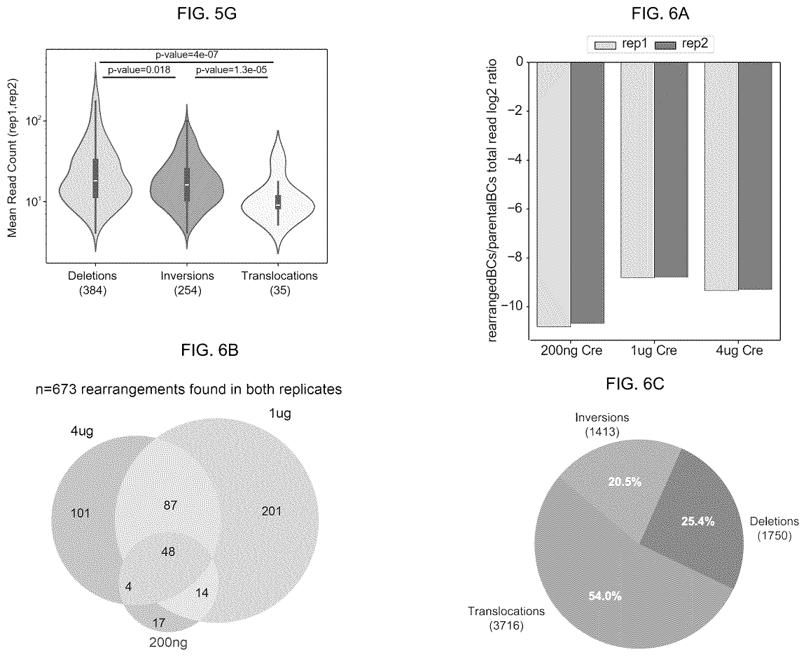

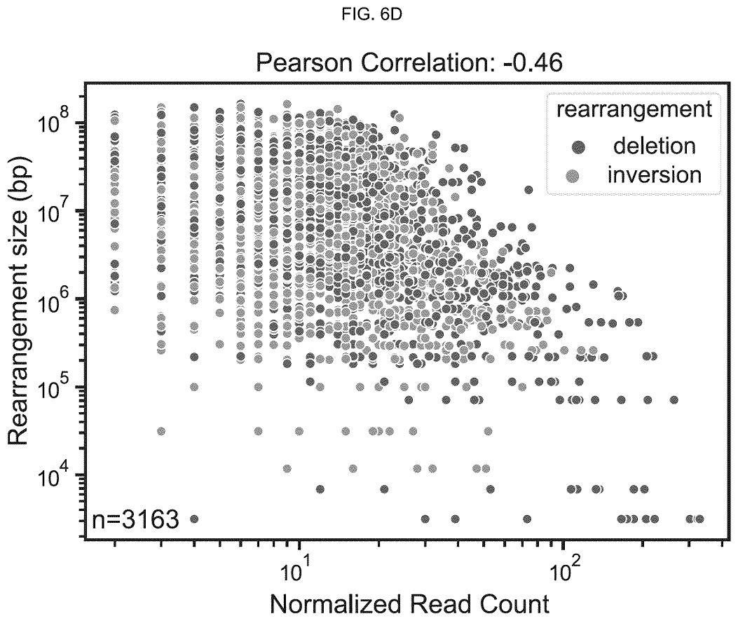

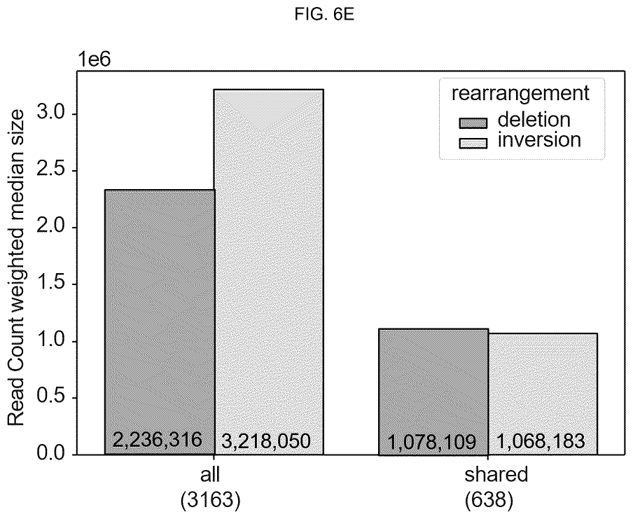

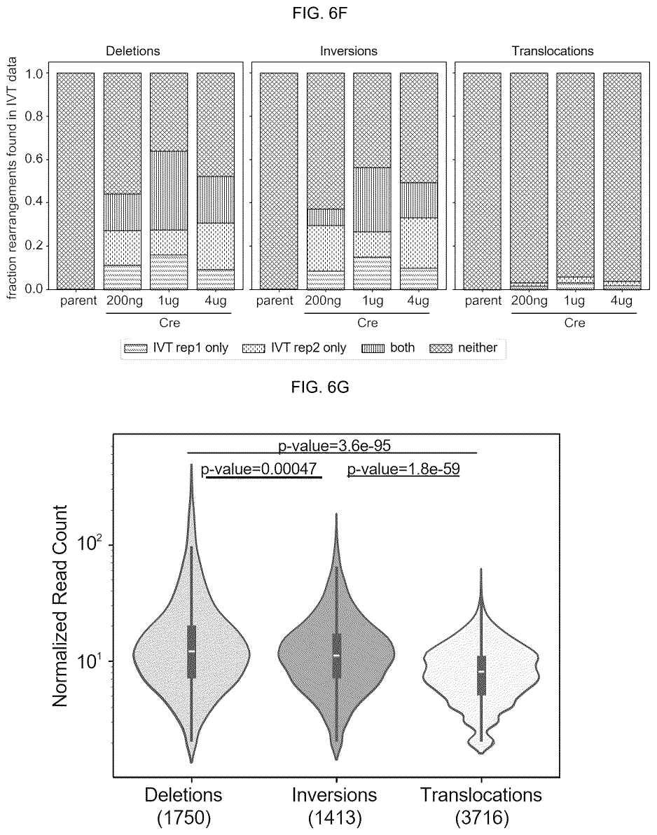

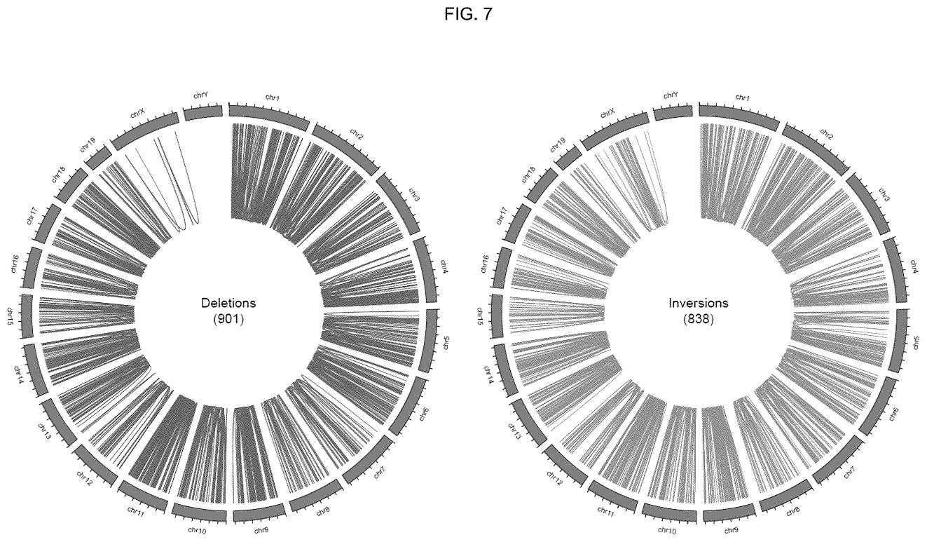

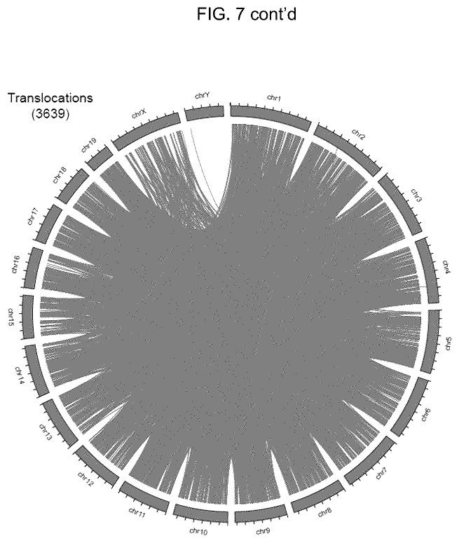

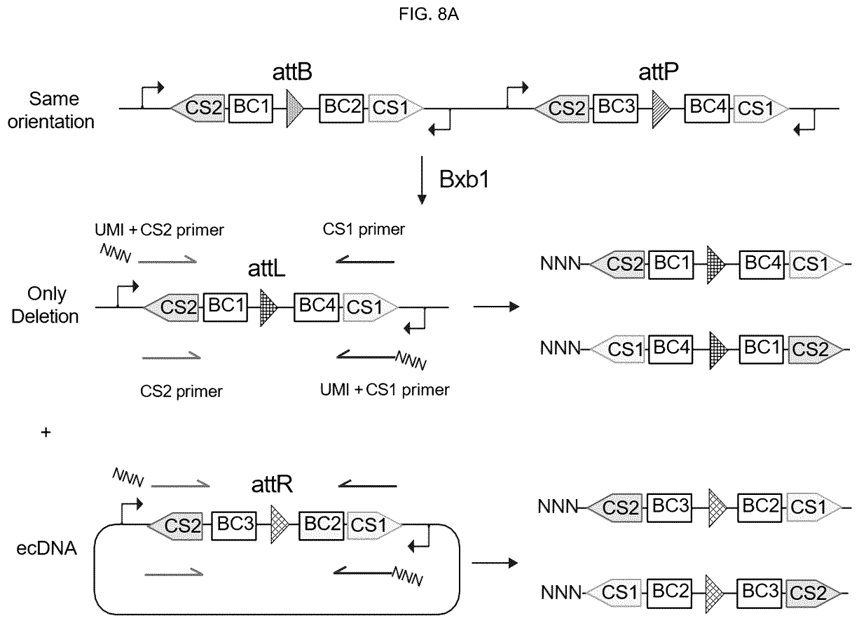

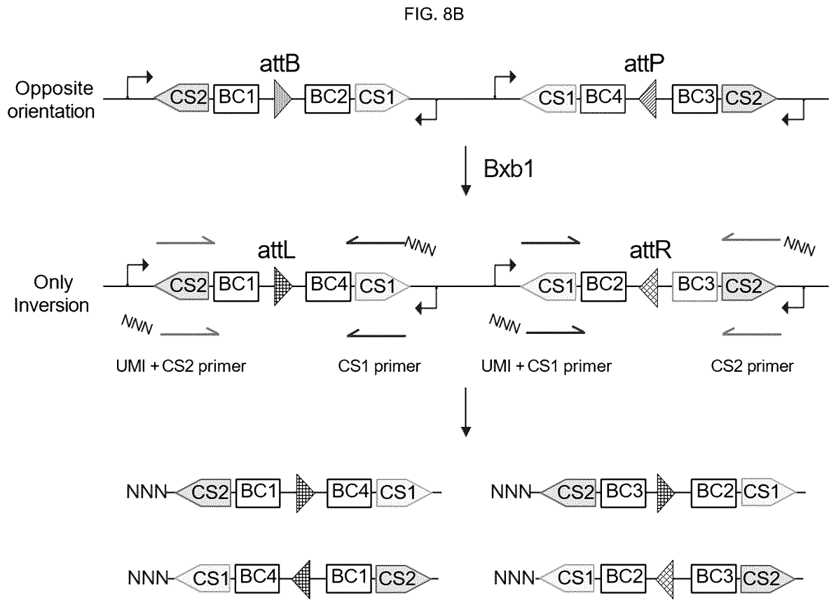

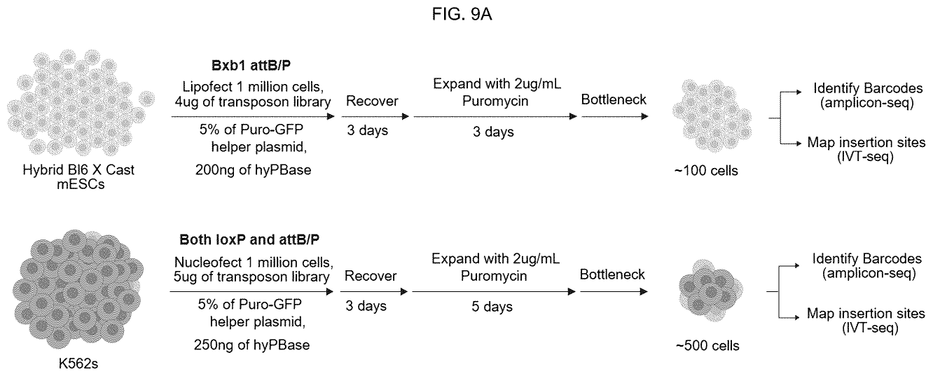

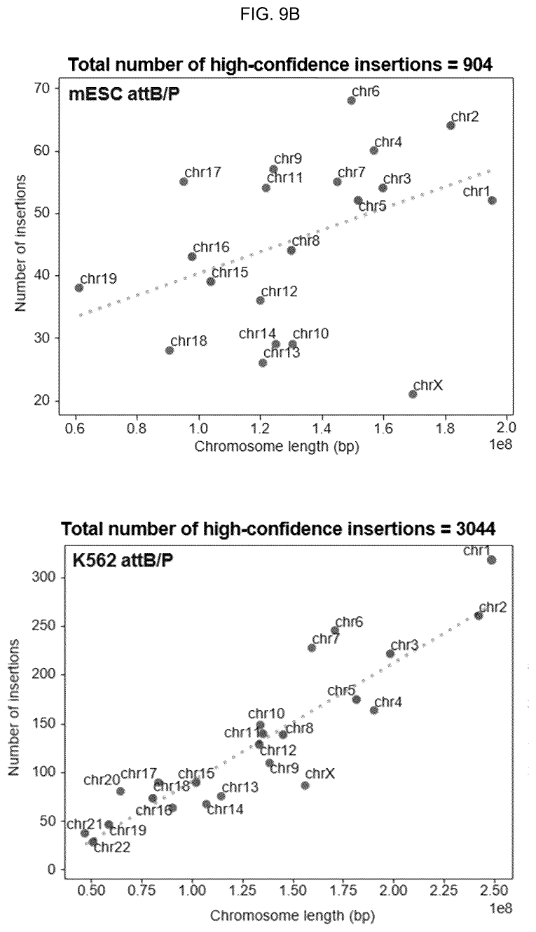

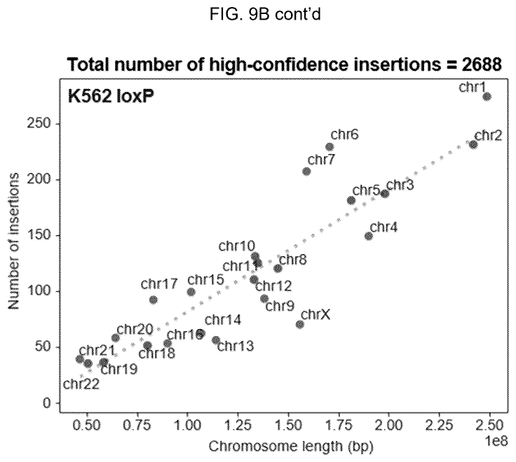

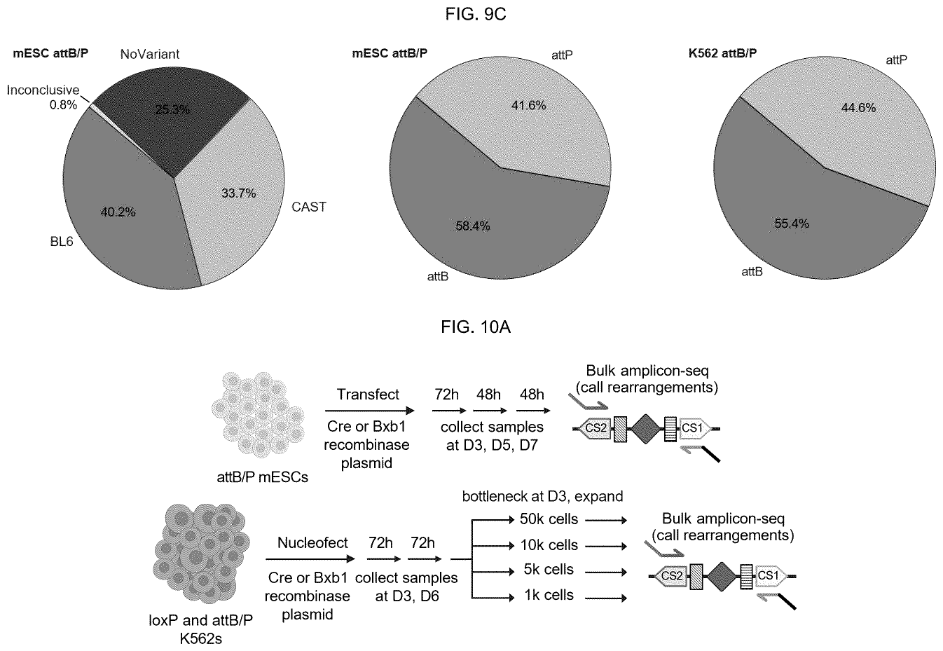

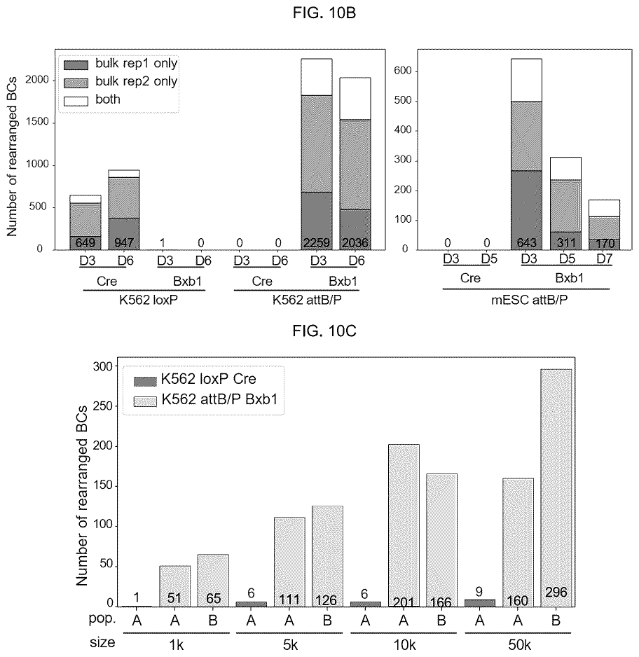

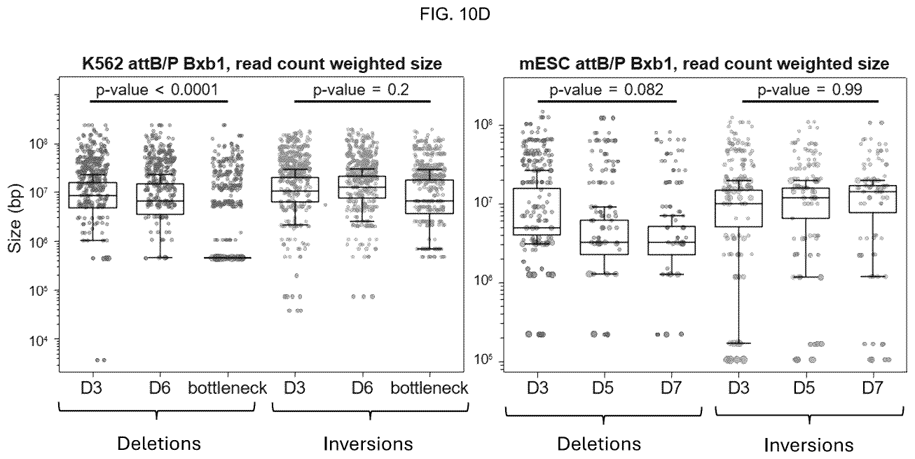

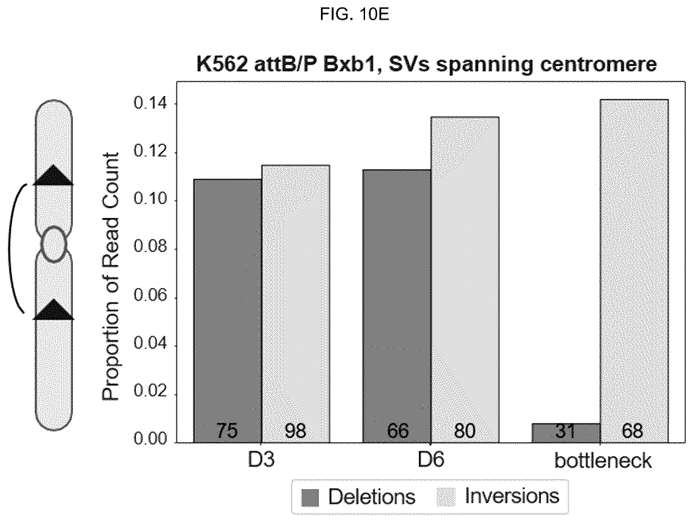

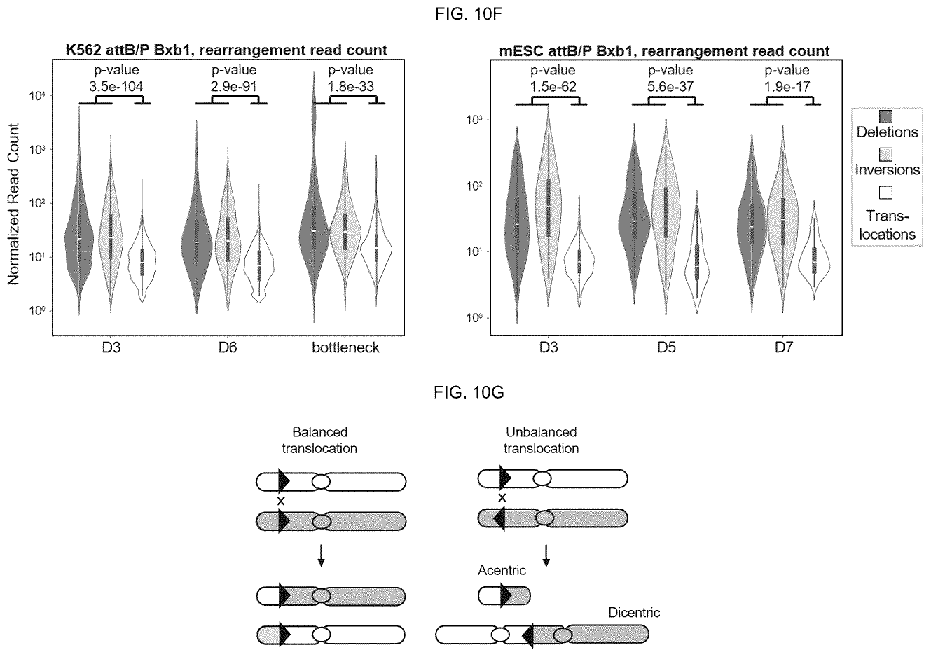

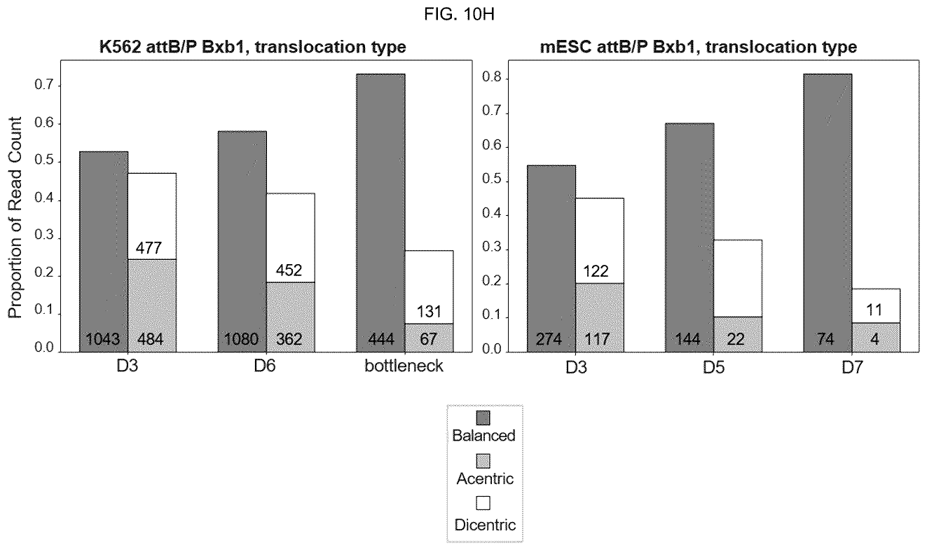

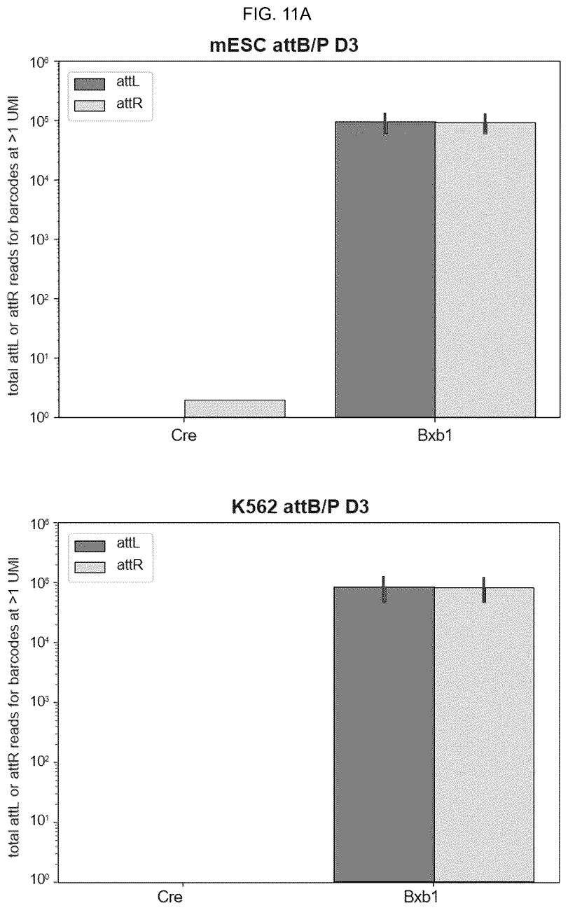

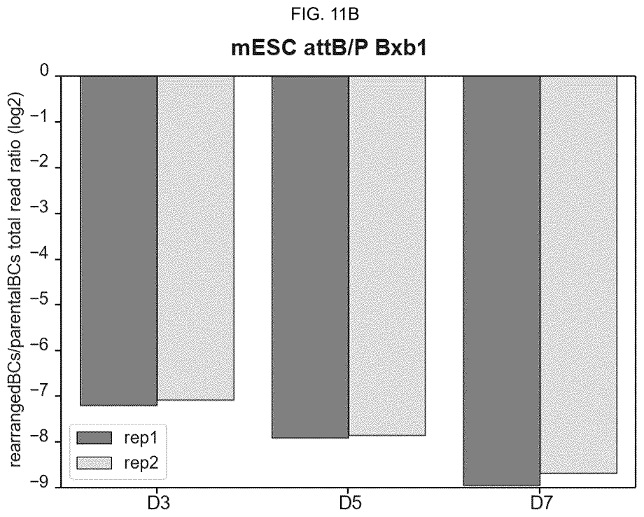

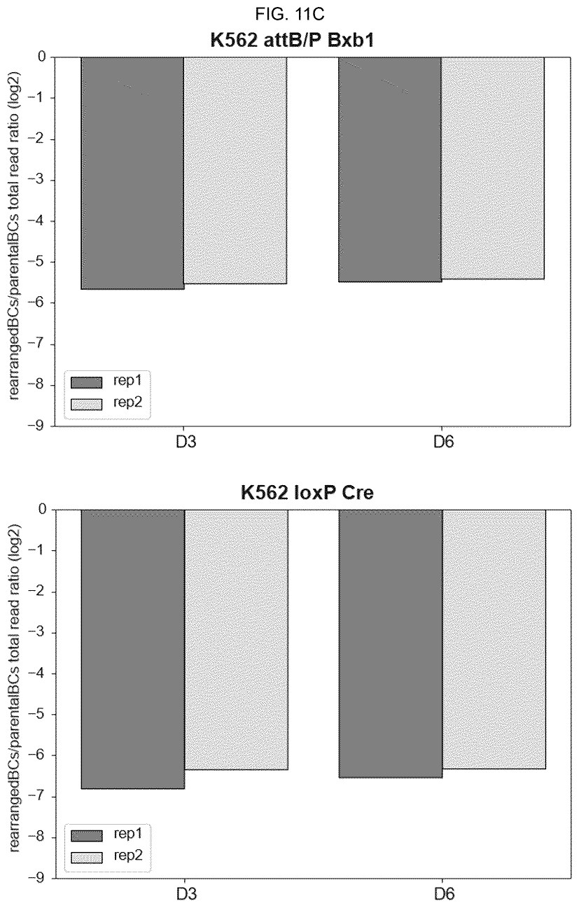

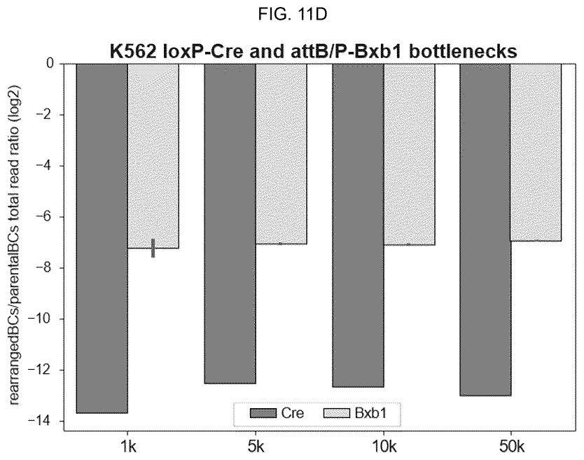

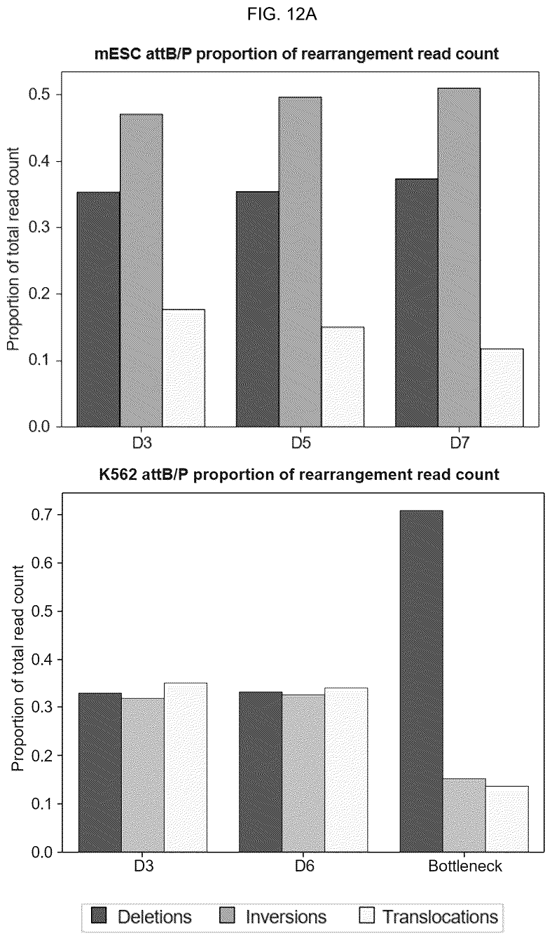

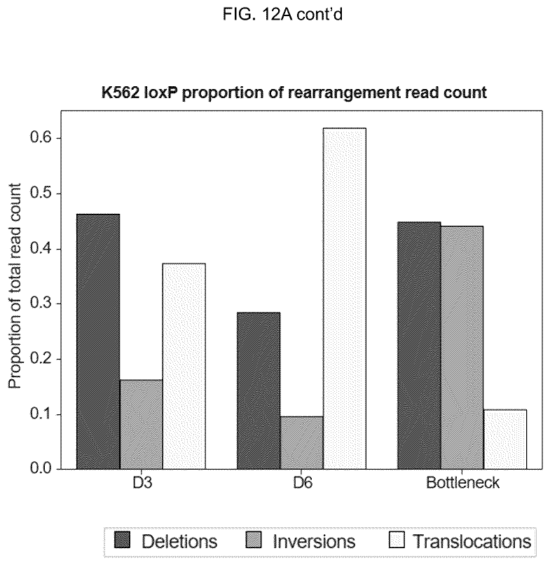

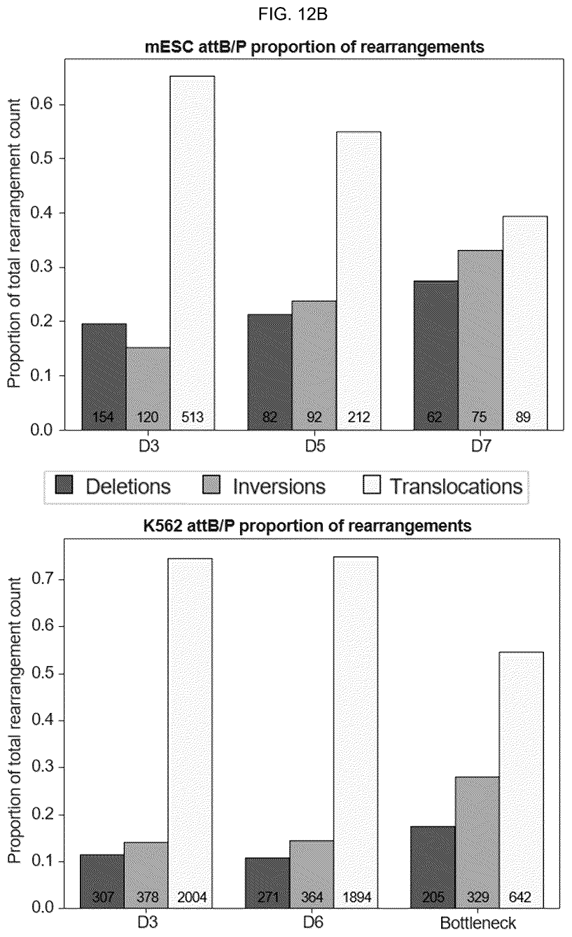

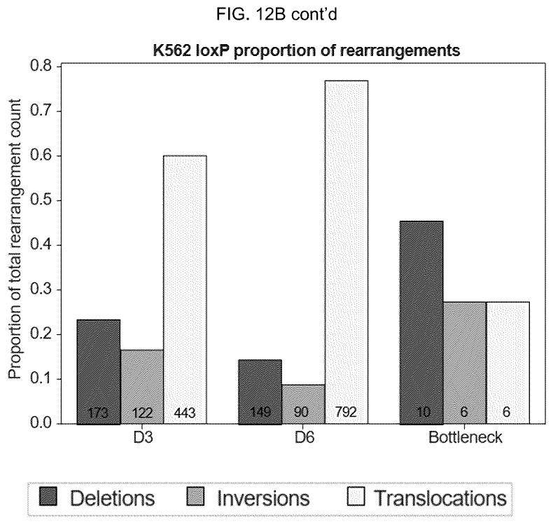

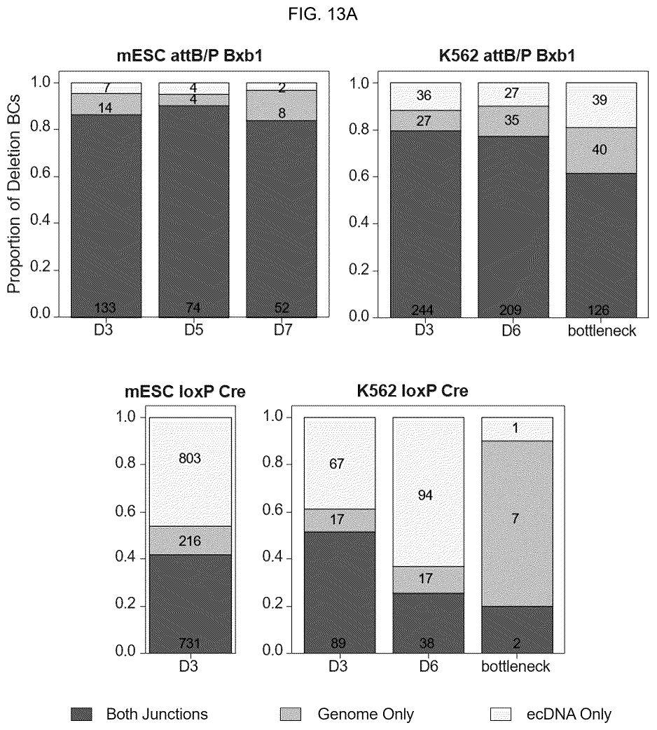

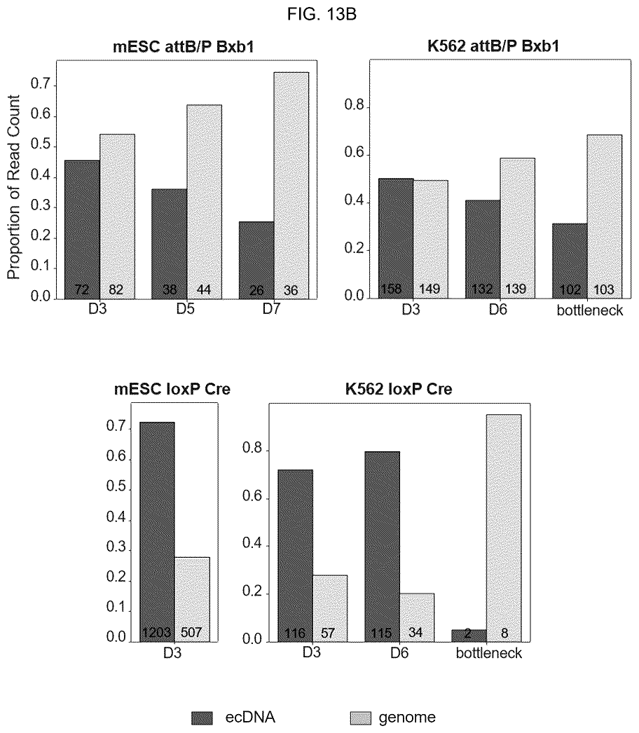

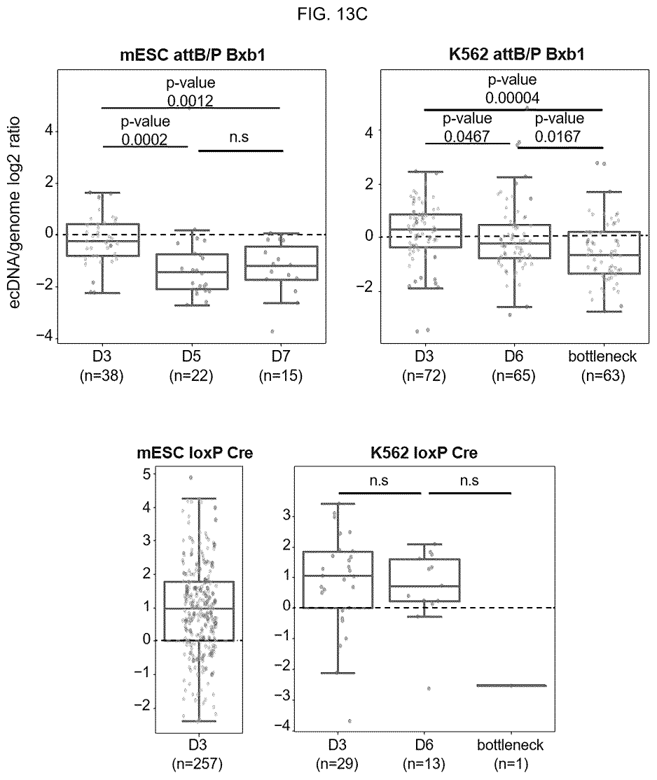

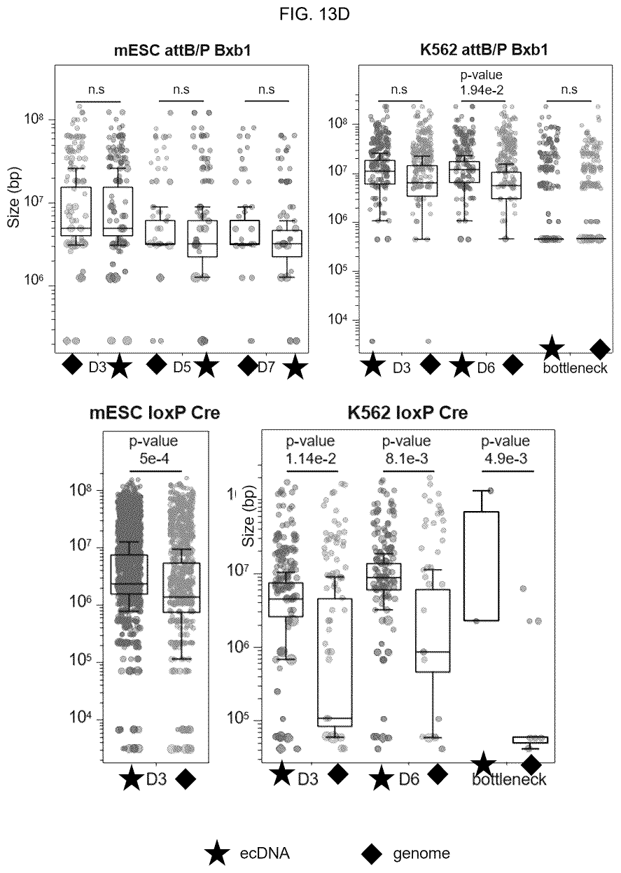

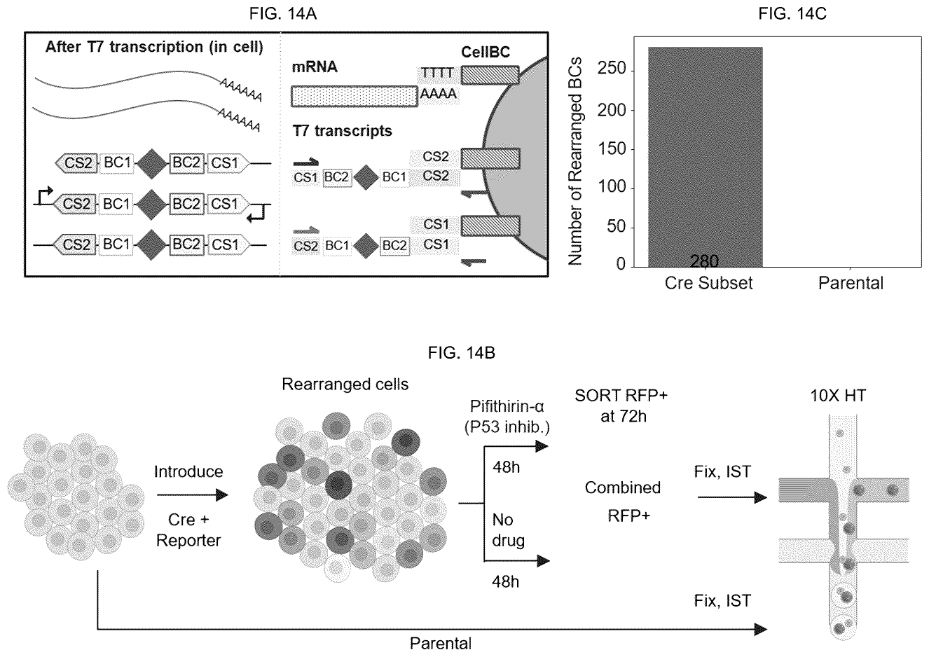

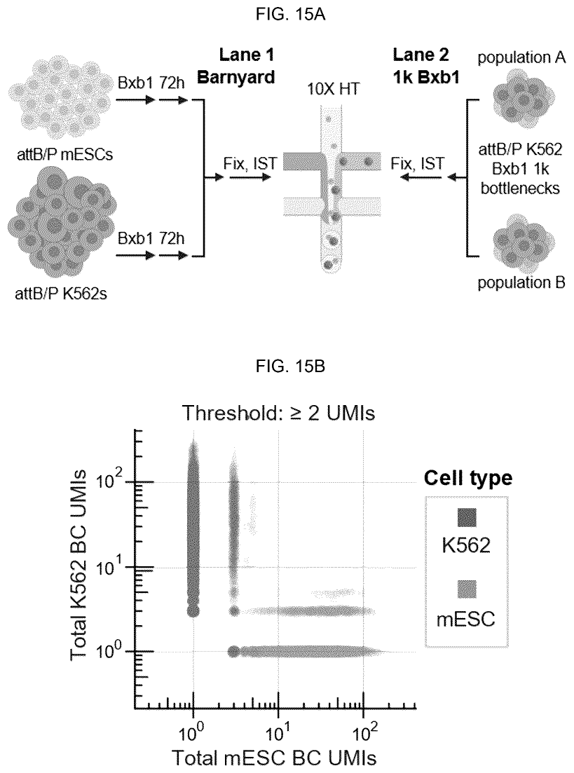

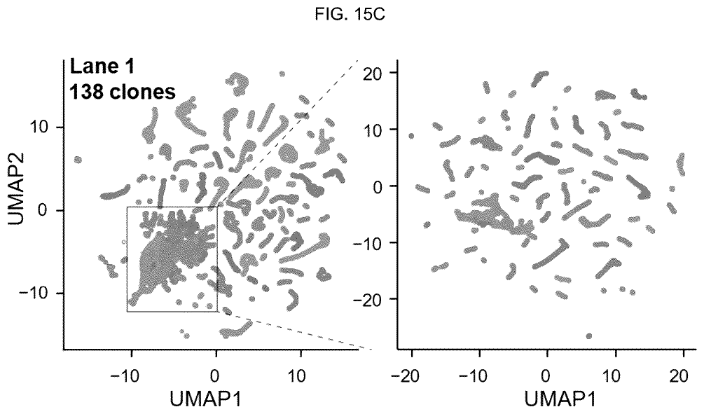

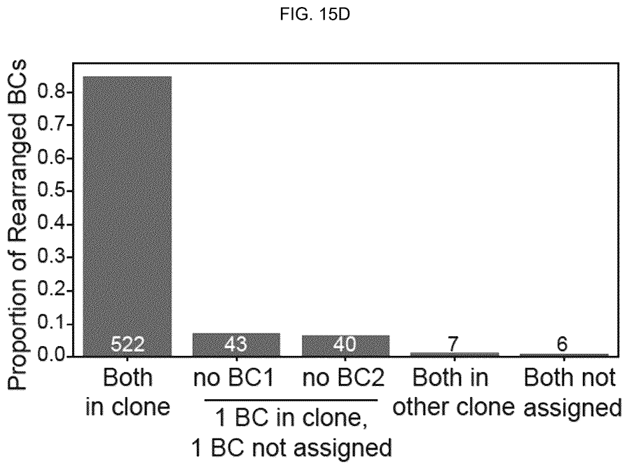

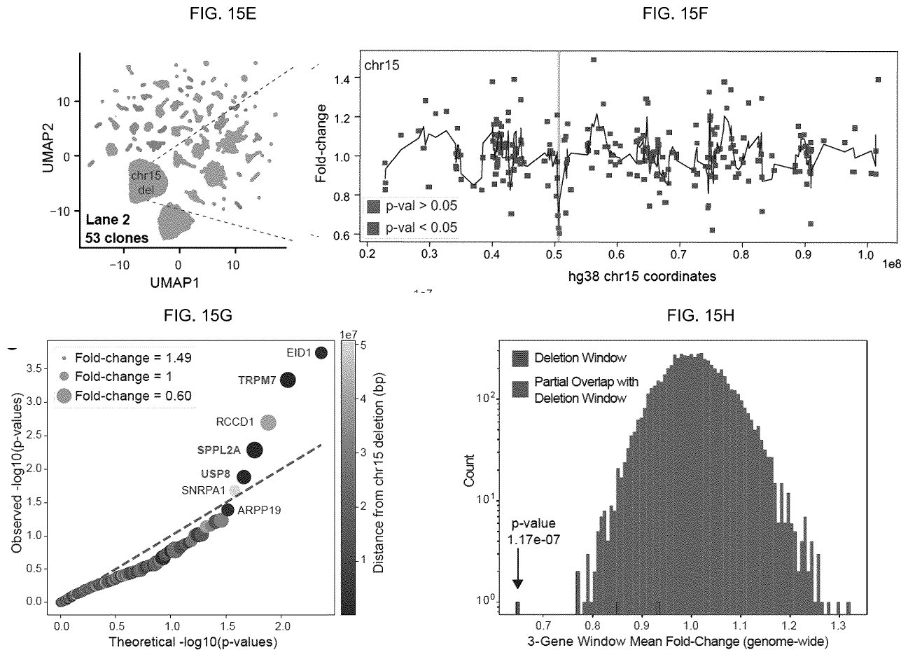

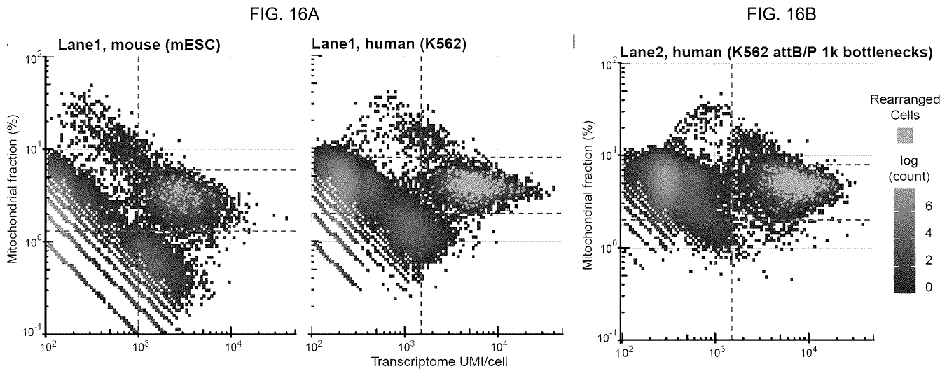

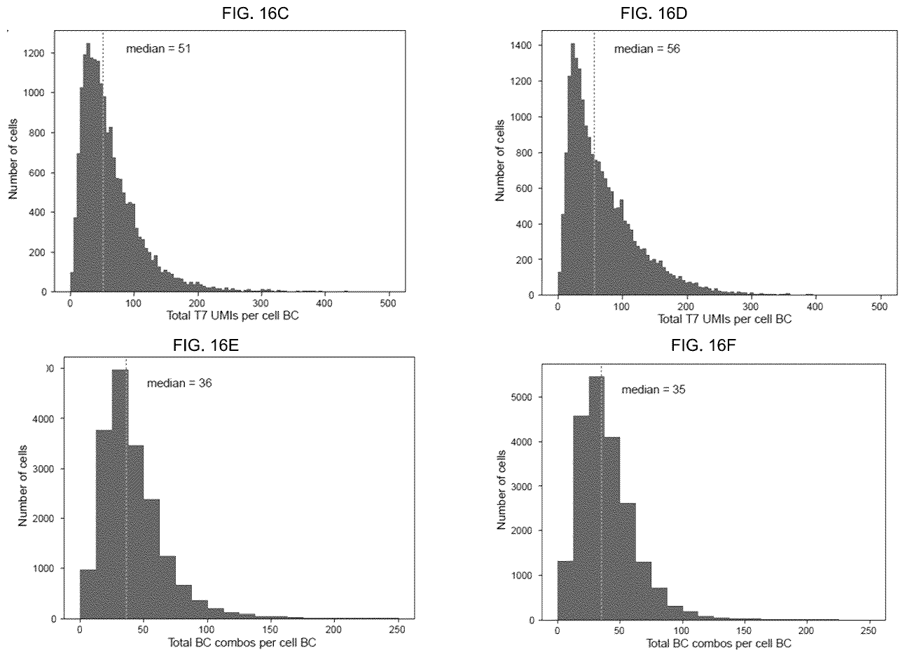

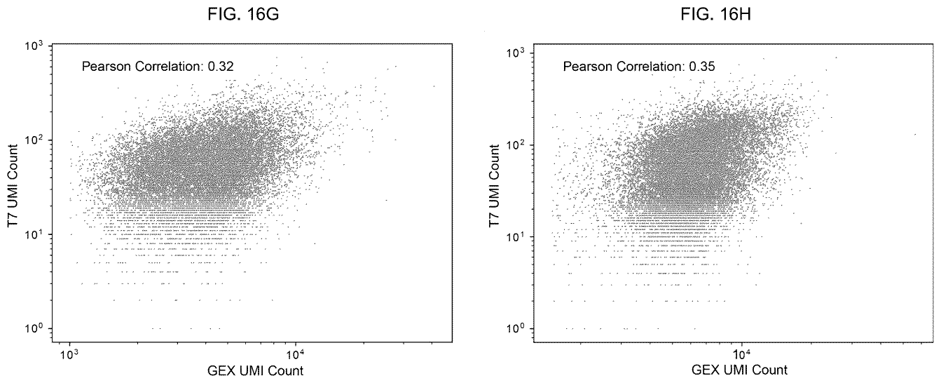

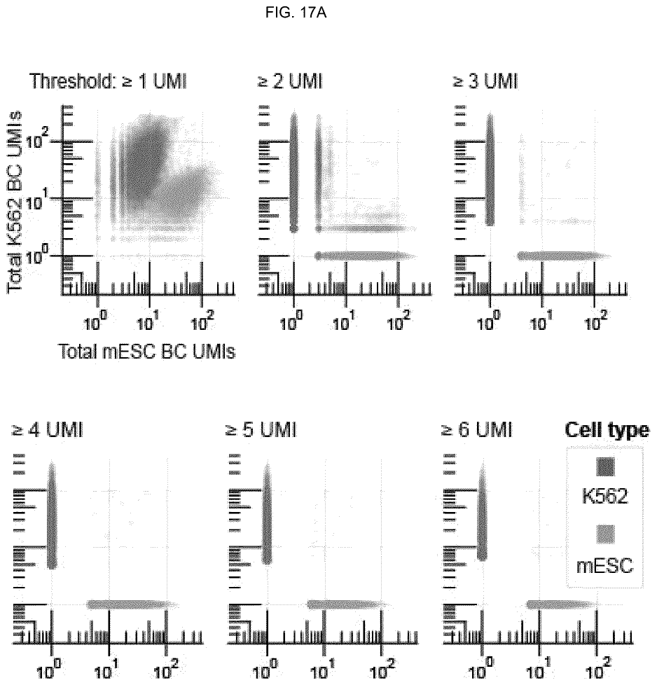

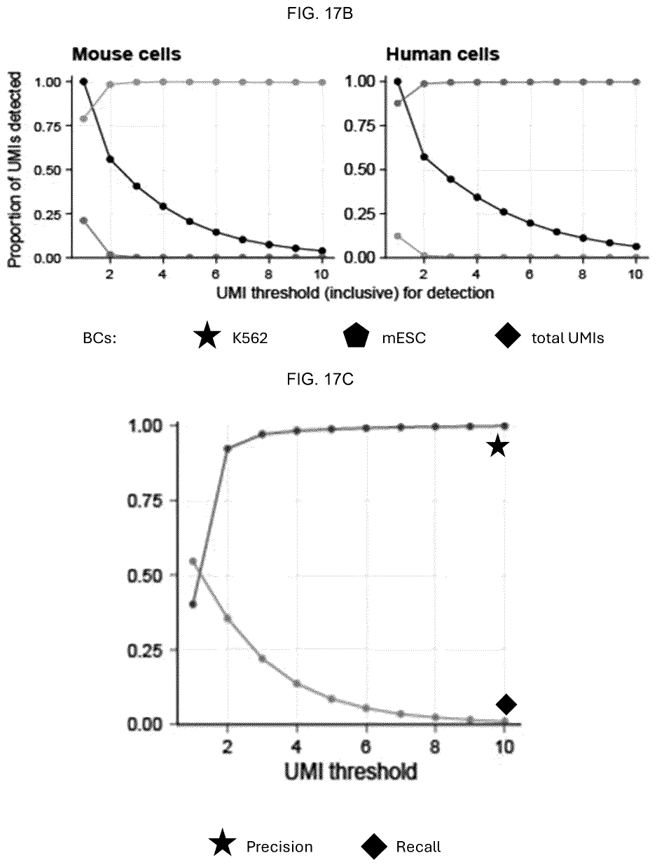

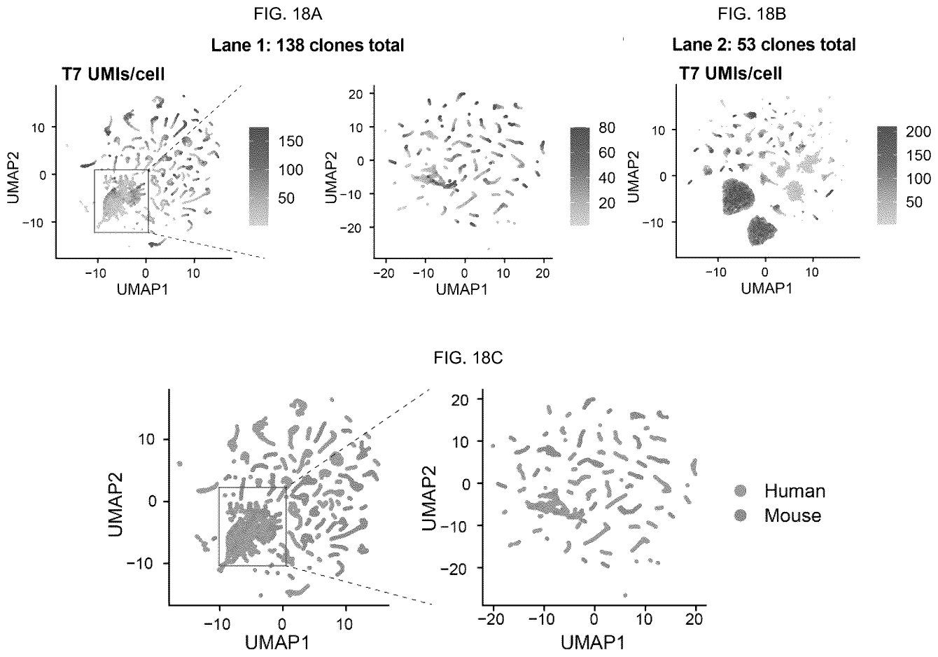

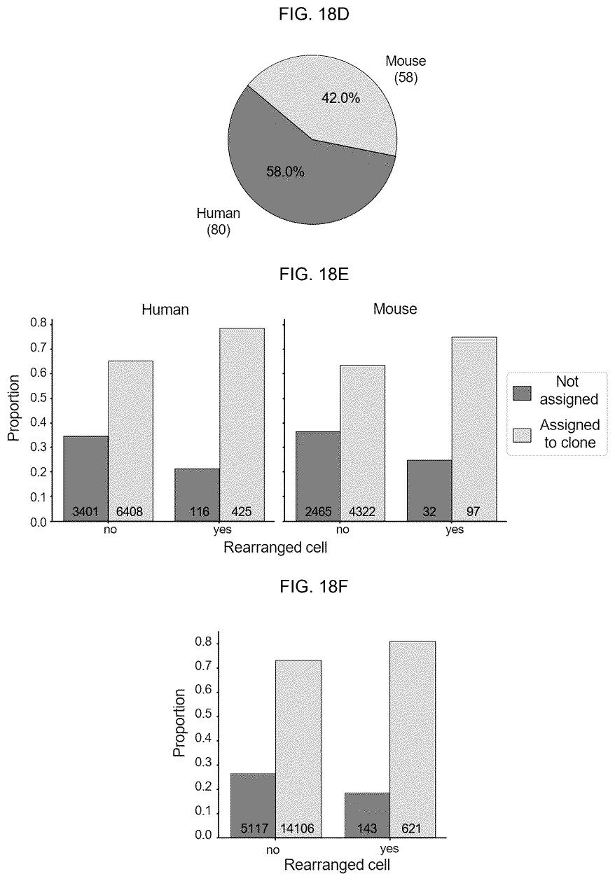

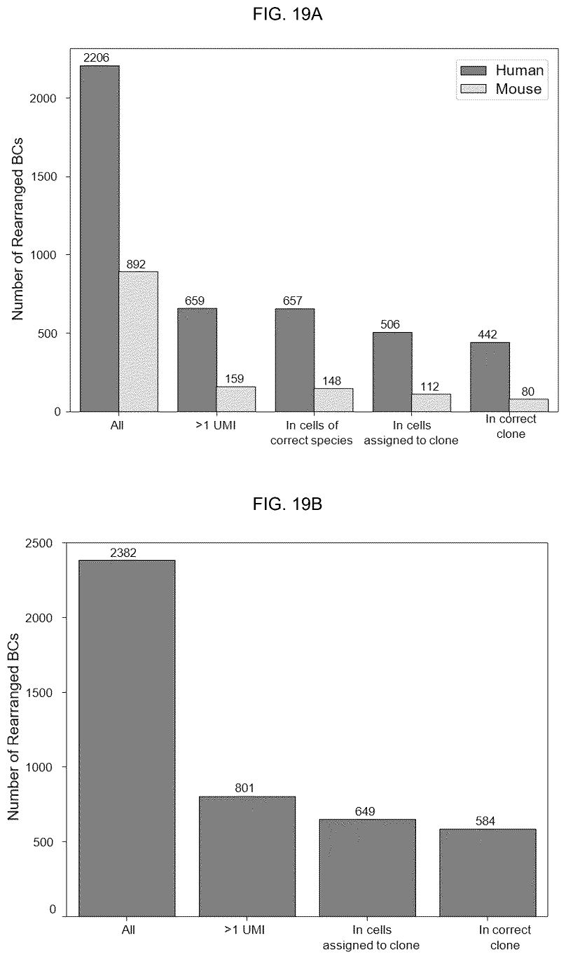

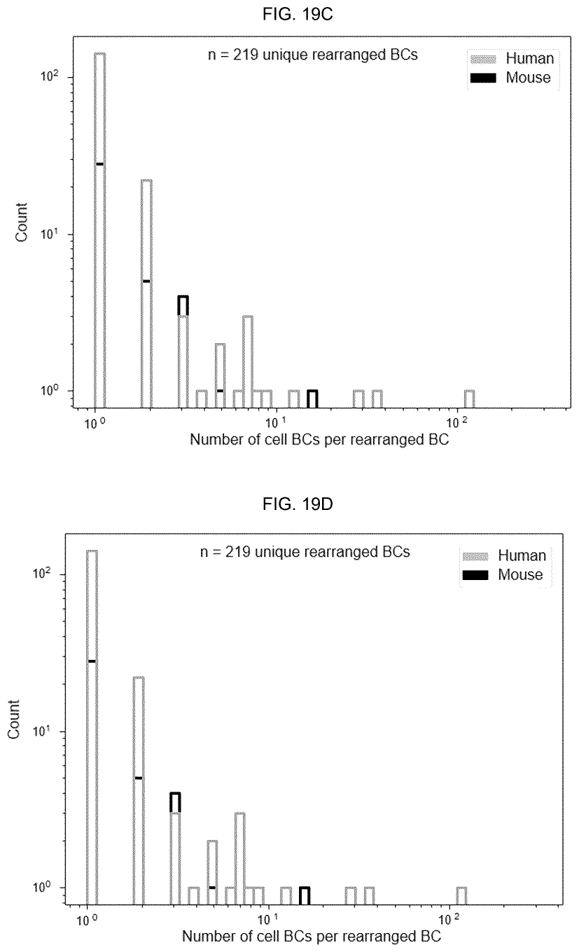

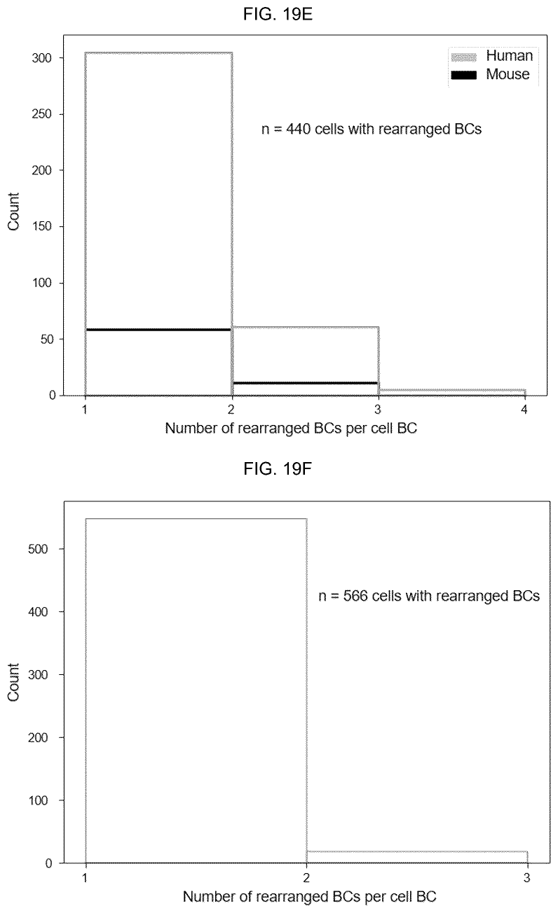

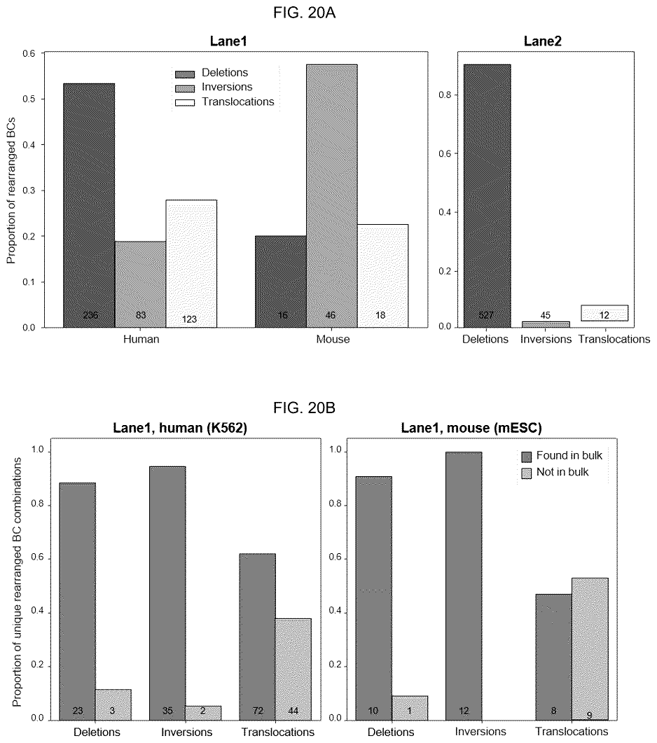

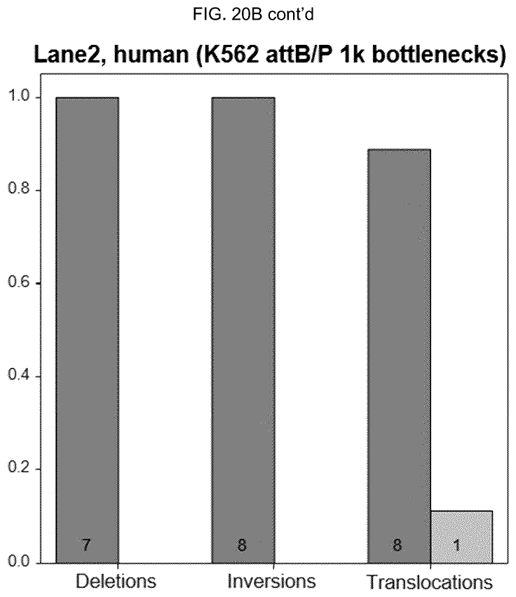

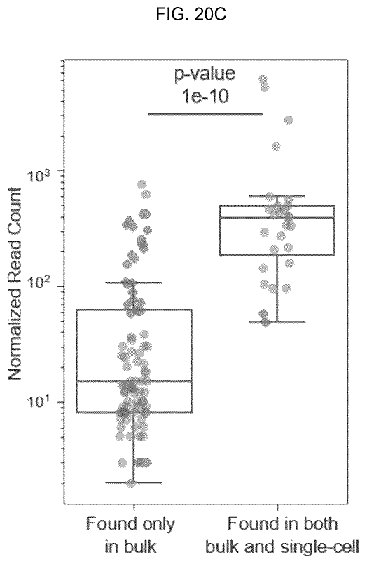

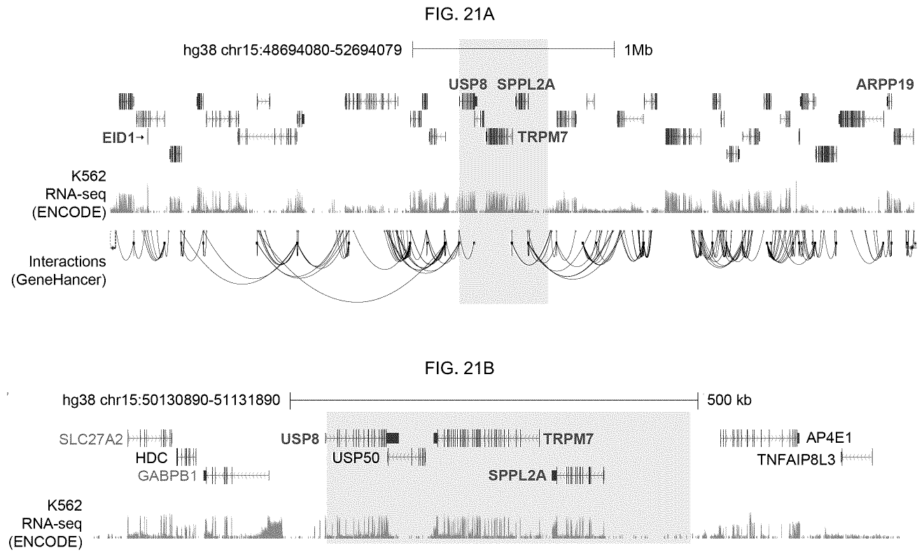

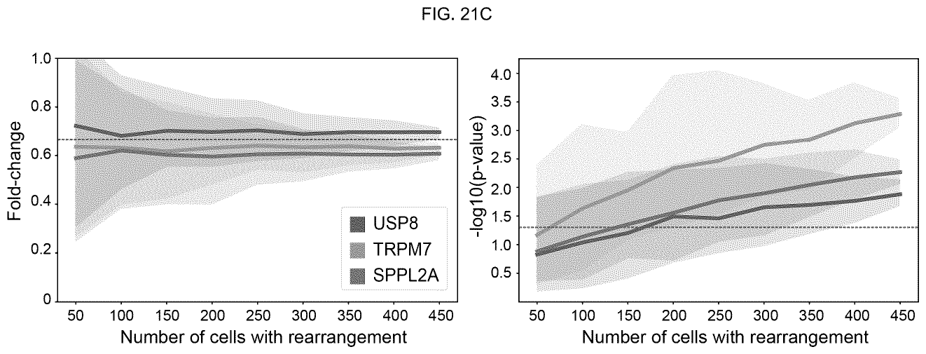

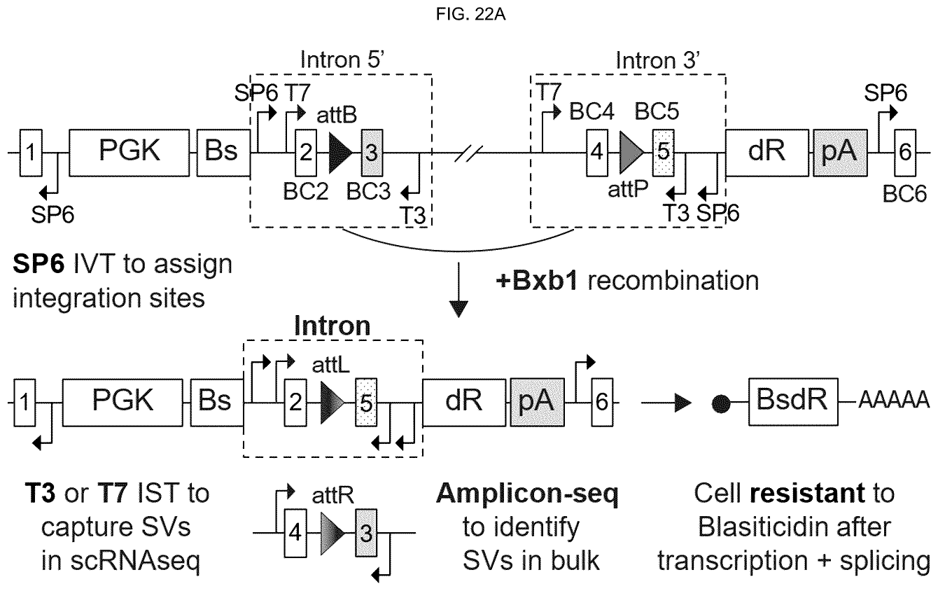

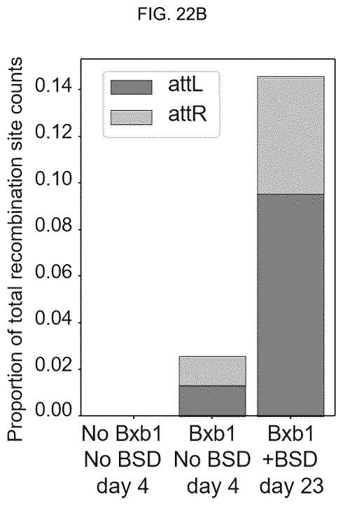

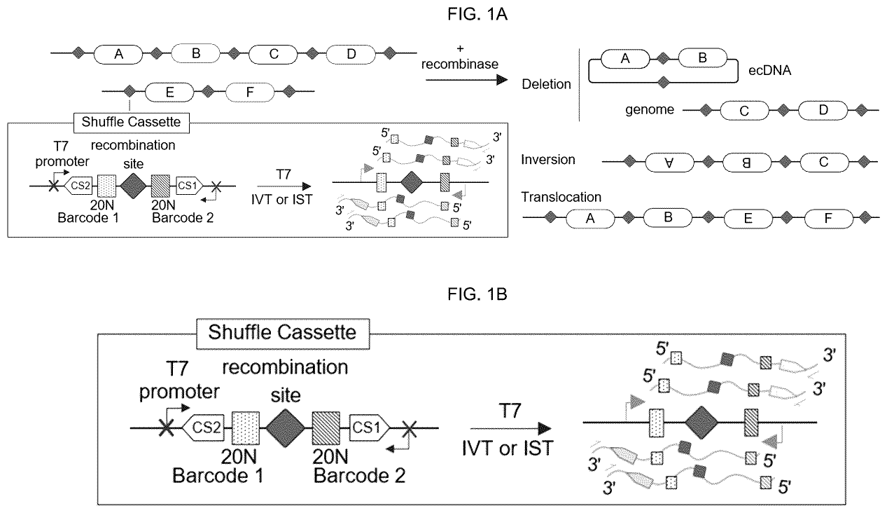

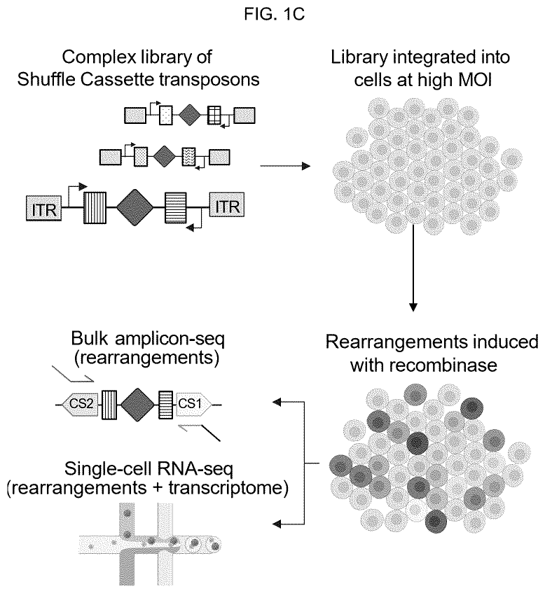

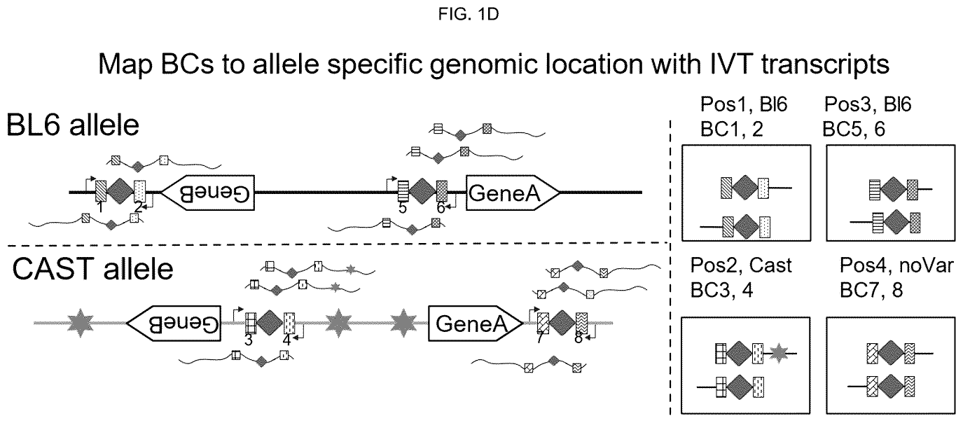

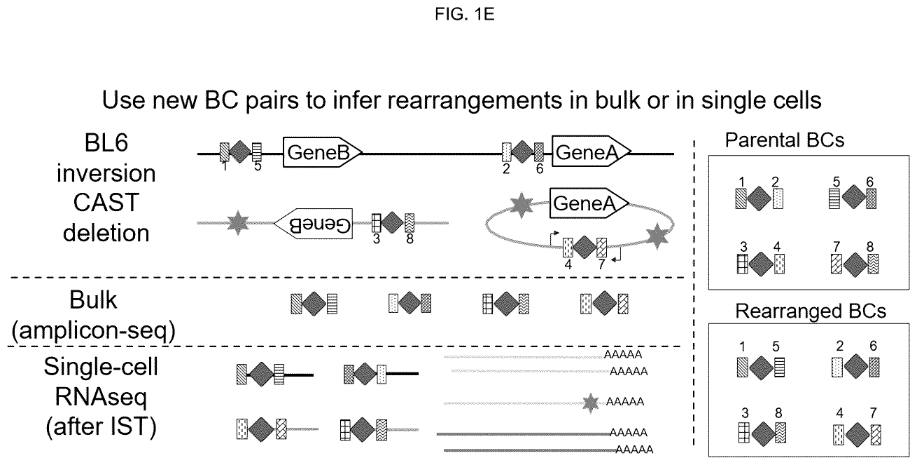

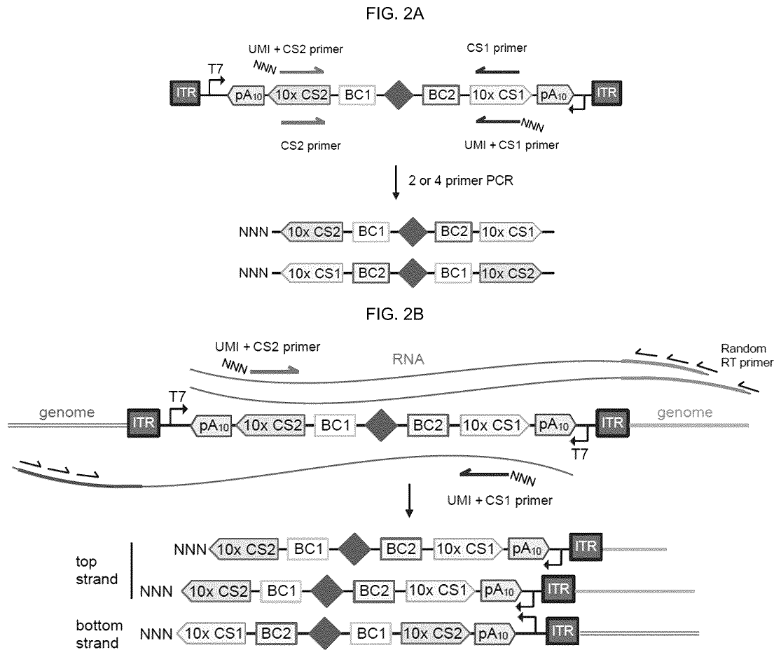

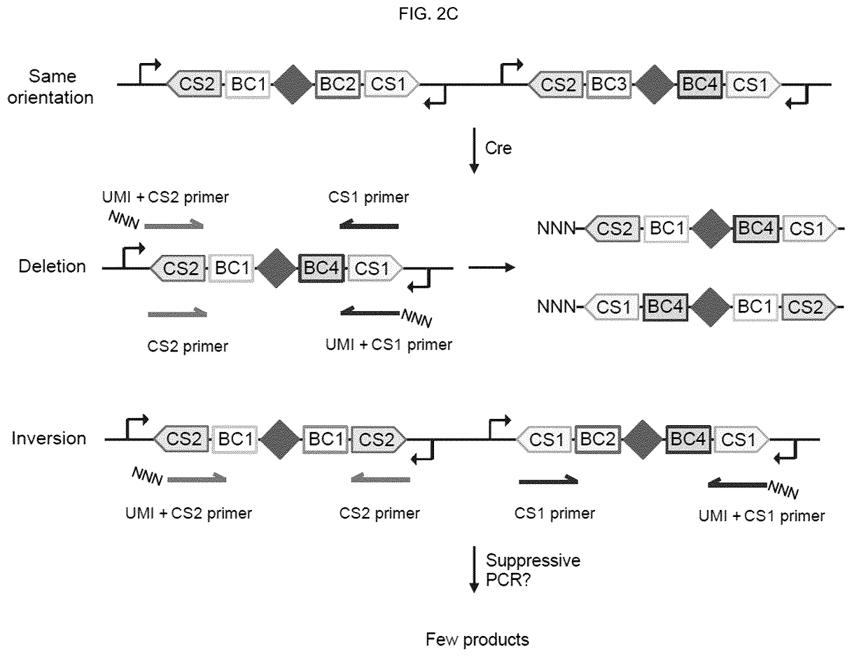

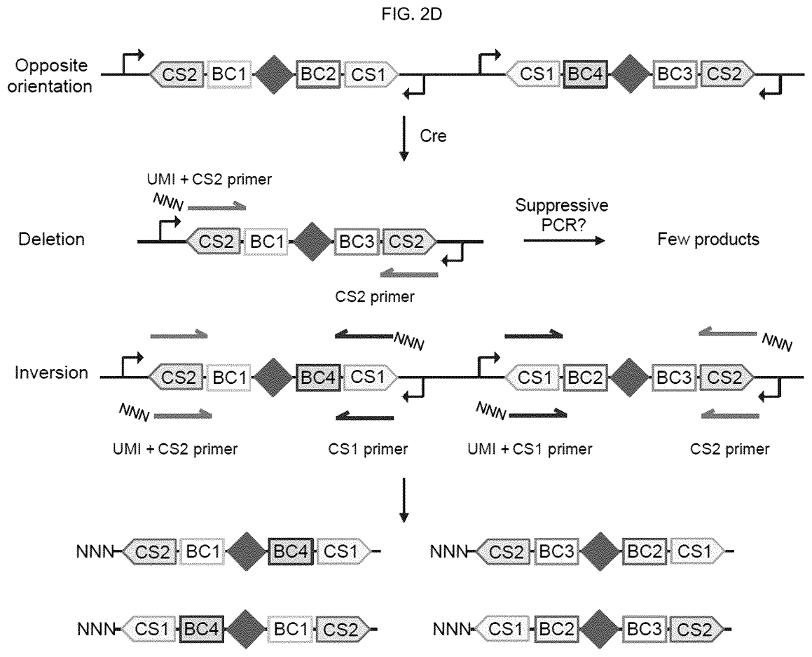

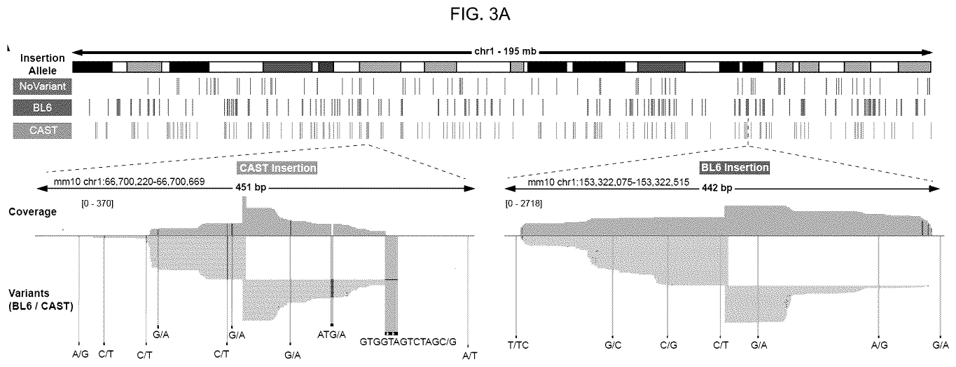

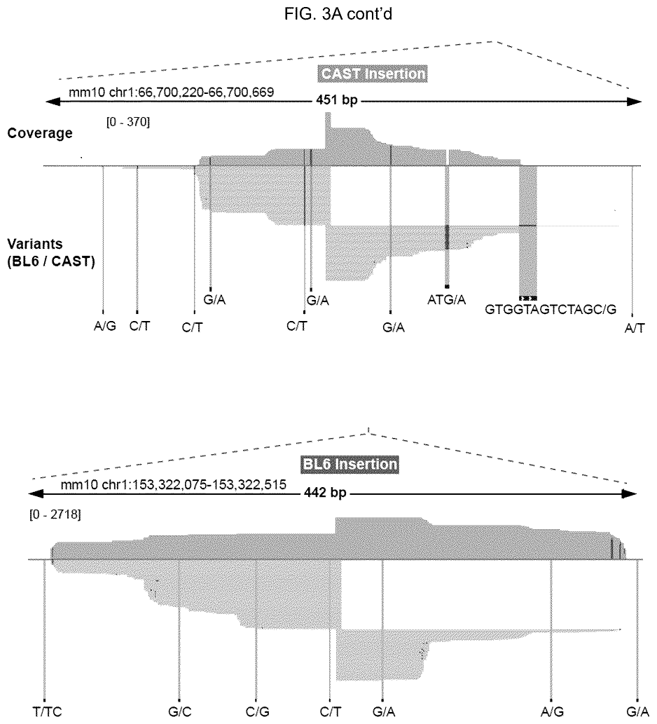

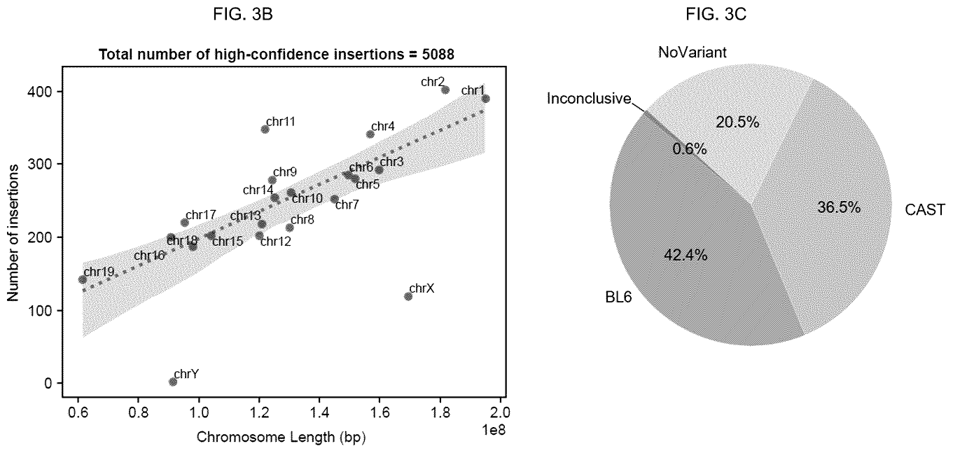

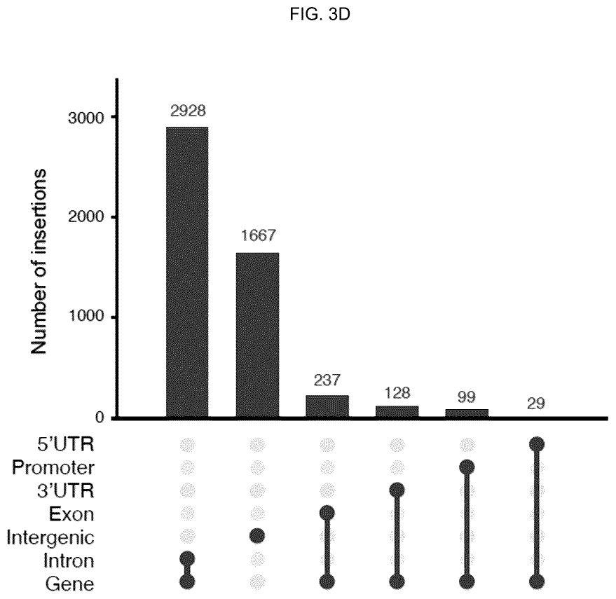

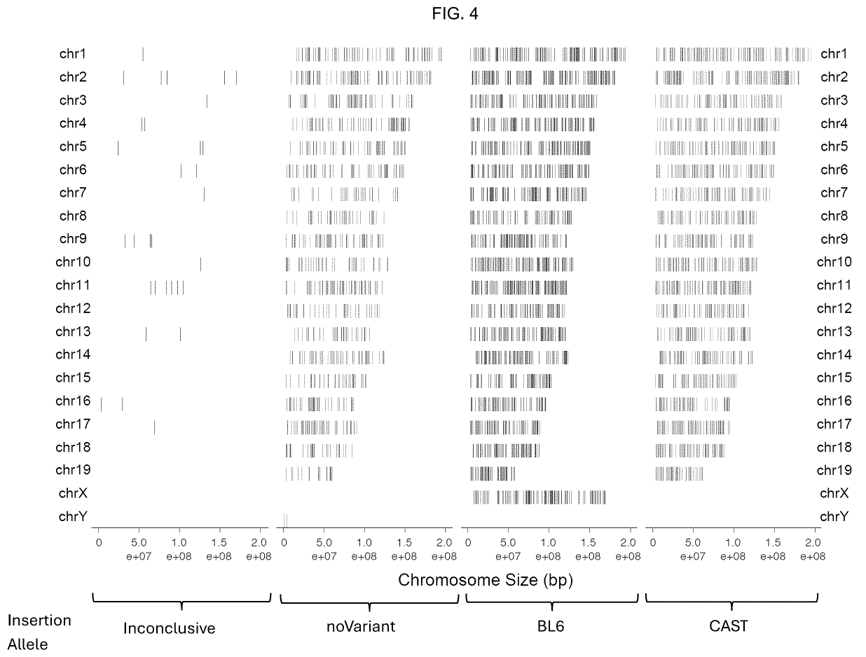

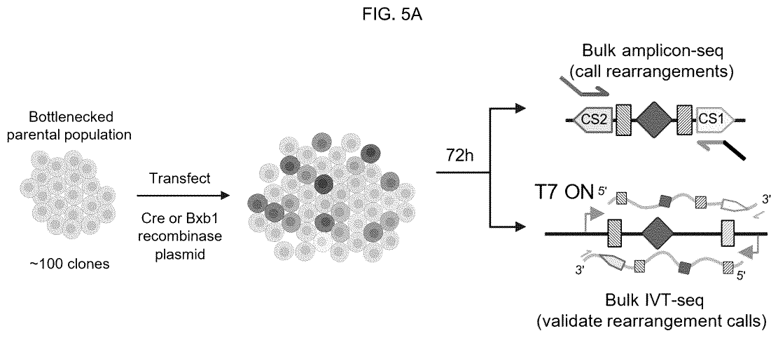

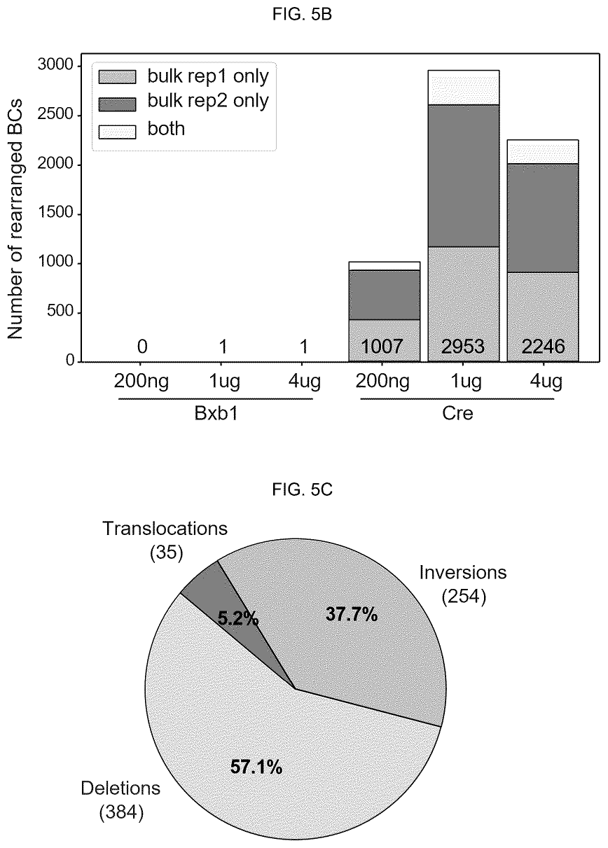

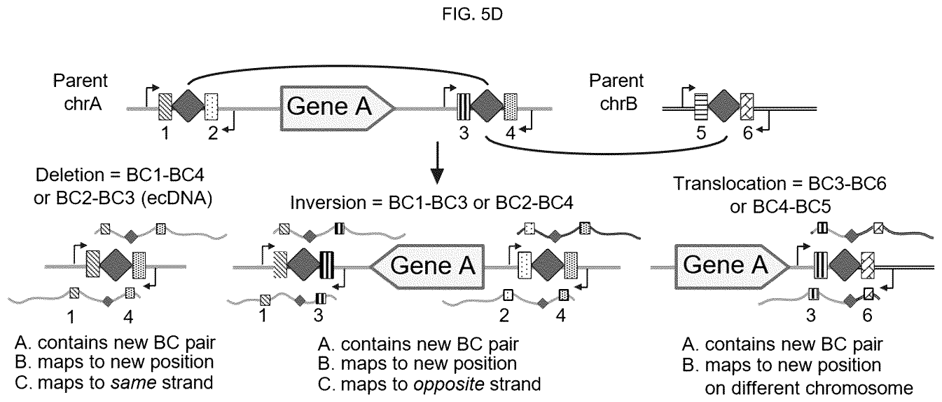

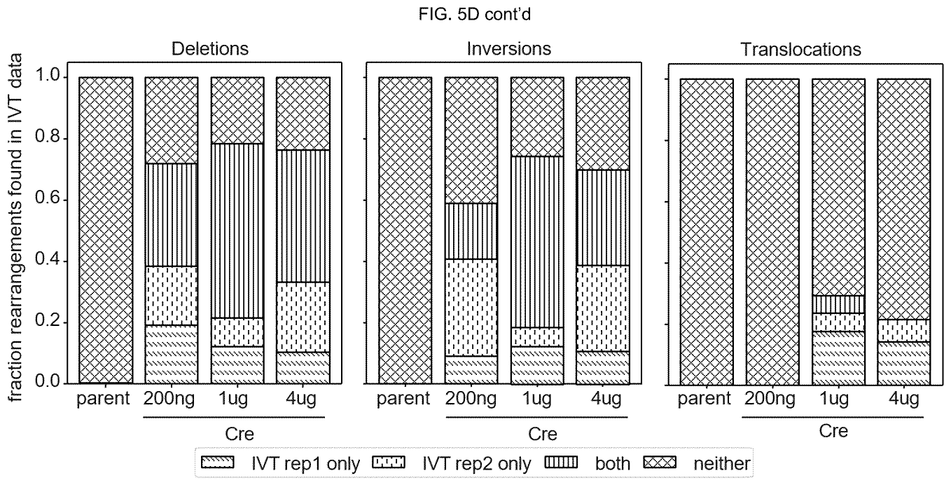

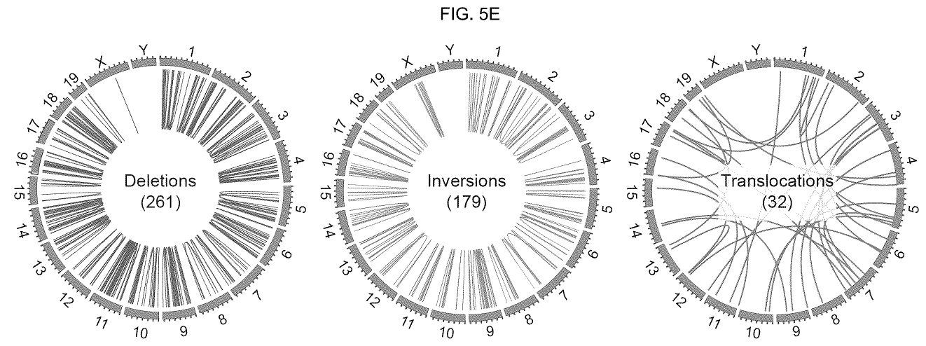

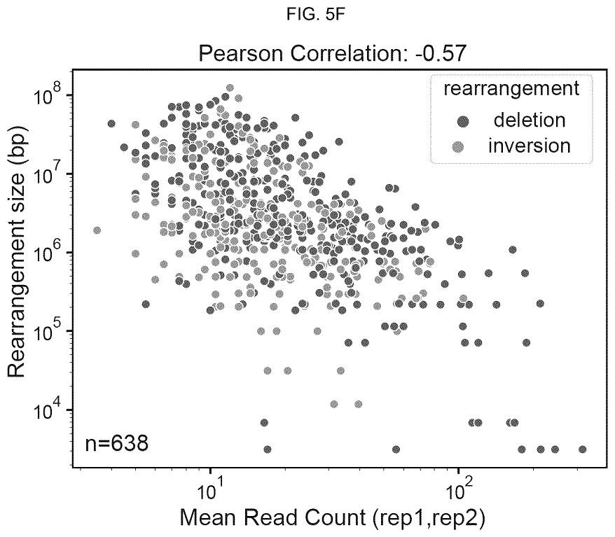

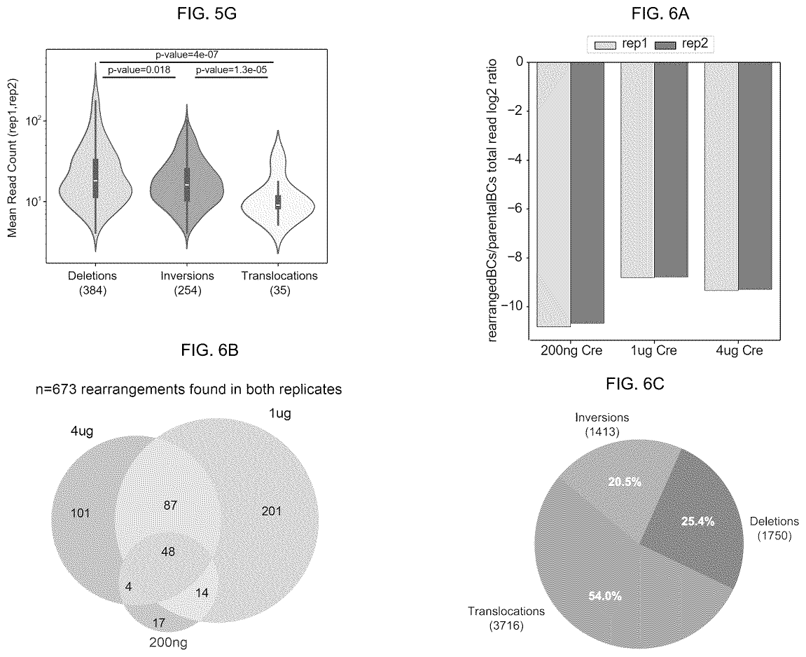

The shuffle cassette library was integrated to BL6xCAST mESC cell line at a high MOI (40). After bottlenecking to 100 founding clones and re-expanding the cell population, an average MOI of 123 was estimated via quantitative PCR (FIGS. 3A, 3B). 9,416 parental barcode combinations were identified in the bottlenecked population by sequencing shuffle cassette-derived PCR amplicons (FIGS. 2A, 3C). T7 IVT-based mapping (37) was performed on genomic DNA extracted from this pool to identify the site of integration and orientation of each shuffle cassette (FIGS. 3A, 3D). After filtering out those mapping ambiguously or to multiple locations, 5,088 barcoded shuffle cassettes were retained whose locations are confidently mapped at base-pair resolution (FIG. 3A). Although well-distributed across the chromosomes, chrX and chrY harbored fewer insertions relative to autosomes, presumably due to their single copy in these male cells and difficulties with mapping on the repetitive chrY (FIG. 3B). Allele-specific SNVs and indels were used to assign 80% of the shuffle cassettes to either the BL6 or CAST haplotype (FIGS. 3C, 5). Shuffle cassettes largely mapped to introns and intergenic regions (FIG. 3D).

Next, to induce SVs, as well as to genotype them via amplicon sequencing of the shuffle cassettes (FIGS. 1A-1E), varying amounts (200 ng, 1 μg or 4 μg) of either a plasmid expressing Cre recombinase or, as a negative control, the non-targeting Bxb1 recombinase, were transfected into cells derived from the bottlenecked population (200,000 cells per condition). At 72 hours post-transfection (day 3), cells were harvested and genomic DNA isolated. Shuffle cassette barcode pairs were then PCR amplified and deeply sequenced, and whether new combinations were present was assessed (FIG. 5A). As desired, while almost no non-parental barcode combinations were detected in the non-targeting Bxb1 recombinase control condition, >5,000 novel barcode combinations were detected across the conditions involving Cre recombinase (FIG. 5B). Because in some cases both non-parental barcode combinations generated by the recombination event (FIGS. 1E, 2C-2D) were detected, these reduce to 4,856 inferred, unique SV events. Of note, 50% of rearrangements between IoxPsym sites are expected to result in shuffle cassettes with the same primer binding site on either side (FIGS. 2C-2D); these may be undetectable due to suppression PCR (41,42).

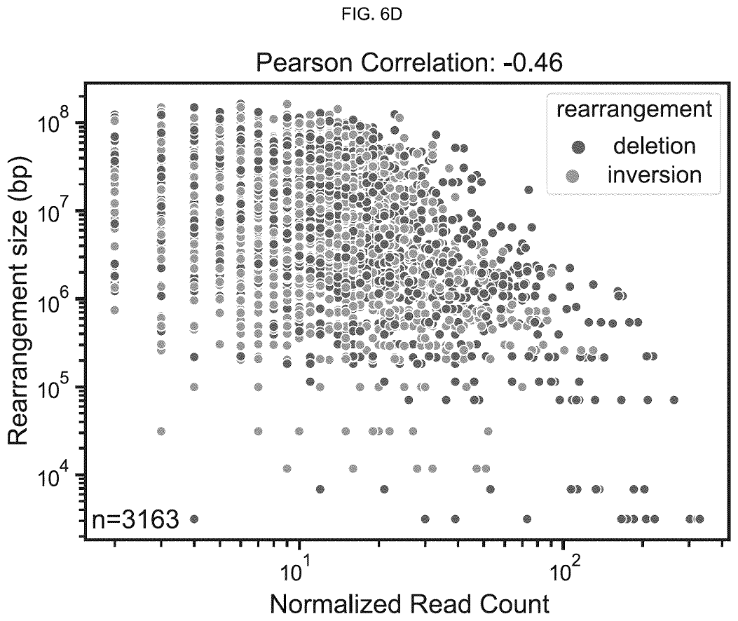

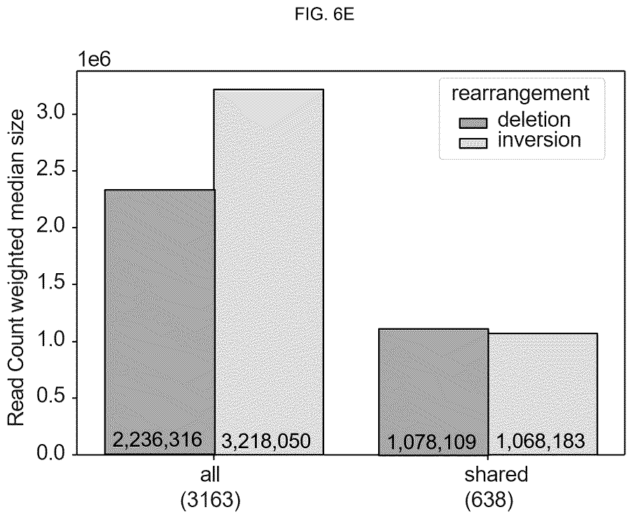

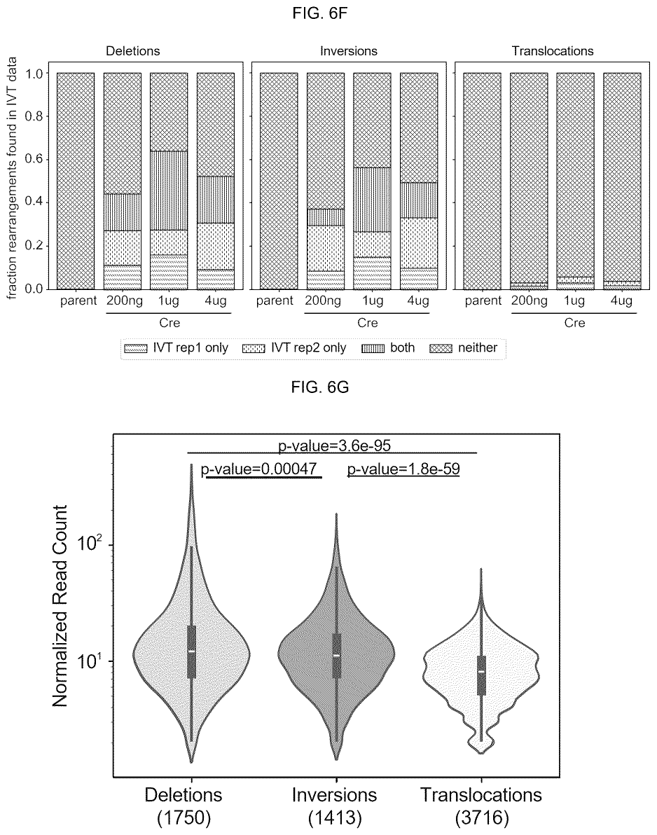

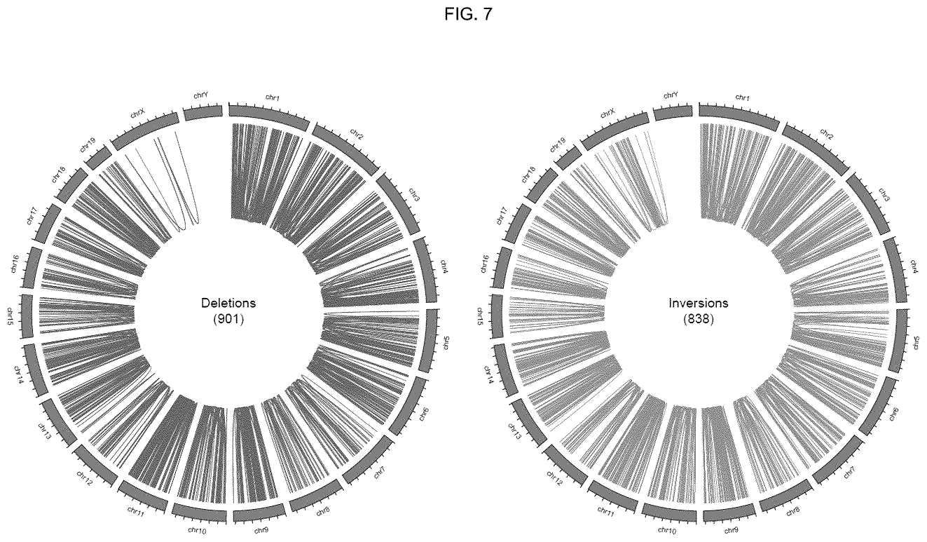

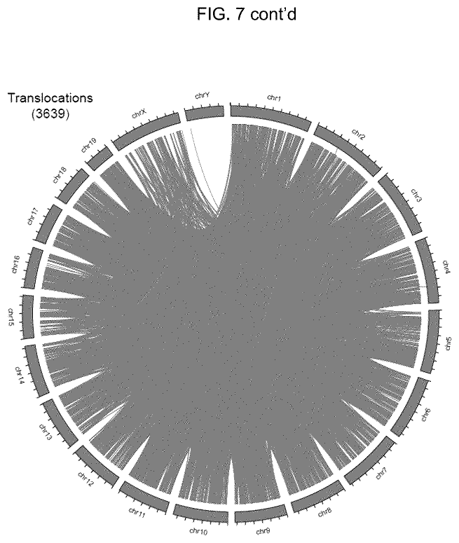

Of note, a small fraction of the SVs that could potentially be generated from these cells is likely to be observed. First, nearly all (99.9%) of amplicons in conditions involving Cre recombinase matched “parental” barcode combinations, consistent with SVs being individually and collectively rare in the cell population (FIG. 6A). Second, the vast majority of novel barcode combinations were not shared between technical replicates prepared from different genomic DNA aliquots from the same Cre condition, nor across Cre conditions. Thus, many more SVs would have likely been detected by processing more Cre-exposed cells from this same population of 100 founding clones (each bearing 50 confidently mapped and oriented shuffle cassette integrations).

Genome-shuffle-seq induces thousands of unique deletions, inversions and translocations. For each novel barcode combination, the class and size of the corresponding SV can be inferred based on the relative genomic coordinates and orientation of the parental shuffle cassettes (FIGS. 1E, 2C-2D). For the subset of SVs shared by both technical replicates of a given Cre transfection condition, 53% were observed in at least one other transfection condition (FIG. 6B). While focusing on SVs observed in both technical replicates of a condition (n=673), deletions and inversions were much more common than translocations (FIG. 5C). However, if detected SVs (n=6879) are considered, translocations comprised the majority (FIG. 6C).

SVs involving the chromosomes were detected, with the exception of chrY (FIGS. 5E, 8, 9A-9H). As expected, the number of SVs detected per chromosome was correlated with chromosome size, presumably a consequence of the number of shuffle cassette insertions. Interestingly, however, when broken down by SV class, some chromosomes appear enriched or depleted for certain classes of rearrangement.

For deletions and inversions, there was an inverse exponential relationship between SV size and abundance, the latter inferred by the number of sequence reads supporting the corresponding barcode combination (FIGS. 5F, 6D). The subset of deletion/inversion SVs supported by both technical replicates (n=638) had a read-counted weighted median event size of 1 Mb, while the complete set (n=3163) had a larger median event size 2.5 Mb. This may reflect the known property of Cre recombination efficiency to drop exponentially with genomic distance in mammalian cells (25) and/or selection acting against those cells containing large genomic deletions or inversions.

To orthogonally validate the SVs inferred from novel barcode combinations, IVT-seq (37) was performed, i.e. the same protocol with which the coordinates of the parental shuffle cassettes were originally mapped, on “post-rearrangement” genomic DNA. Given the inward orientation of the T7 sites, IVT transcripts from each T7 site is expected to cover not only the novel barcode combination, but also flanking genomic DNA, thereby facilitating direct verification of the corresponding SV (FIG. 1B). For deletion/inversion SVs, a substantial portion (40-80%) of either the technically replicating (n=638) or full (n=3163) sets of deletions were validated by at least one IVT-seq read from the same condition, but entirely unsupported by IVT-seq data from parental cells (FIGS. 5D, 6F). In contrast, despite the fact that translocations made up the majority of the detected SVs, far fewer translocations (5-30%) of translocations were supported by IVT-seq data (FIGS. 5D, 6F). Consistent with that, in the amplicon sequencing of shuffle cassettes that originally detected each SV, translocations were supported by substantially fewer reads than deletions or inversions (FIGS. 5G, 6G). Artifactual explanations such as chimeric PCR are ruled out by the complete absence of reads supporting any type of SV, including translocations, in Bxb1-treated control cells (FIG. 5B). One potential explanation of both lower abundances and lower validation rates for translocations is that many detected deletions and inversions are being generated recurrently even within a single condition/replicate (i.e. in independent cells), while detected translocations occur uniquely, precluding validation in independent aliquots of “post-rearrangement” genomic DNA and lowering read counts within the aliquot in which they occurred. An alternative explanation is that translocations are occurring at similar rates but being strongly selected against, either indirectly (via generalized Cre toxicity) or directly (via phenotypic consequences of the translocation itself).

Taken together, these results show the ability to induce, detect, quantify and characterize thousands of deletions, inversions, and translocations in a pool of cells in a single multiplex experiment with Genome-Shuffle-seq, without any single-cell cloning, genotyping or whole genome sequencing.

SVs mediated by Cre at symmetric recognition sites are rapidly depleted from in vitro mESCs. To evaluate the stability of Genome-Shuffle-seq induced SVs, the population of Cre-transfected cells was sampled at days 5 and 7 post-Cre transfection, and sequenced shuffle cassette-derived amplicons to analyze the fate of cells induced to generate SVs on day 3 (Methods). A sharp decline in the number of detected SVs at later time points, with almost no SVs detected by day 7. This could be due to the well-documented toxicity of Cre recombinase to mammalian cells, which is thought to impose cost on cell fitness in proportion to the number of target sites in the genome, in a p53-dependent manner (43-45). The impact of this generalized Cre toxicity on transfected cells would lead to untransfected or poorly transfected cells overtaking the population. As a potential solution, tamoxifen-inducible Cre variants (CreERT2 and ERT2CreERT2) could be used to restrict the time window of Cre activity, limiting toxicity (43,46). To test this, bottlenecked parental cells with inducible Cre variants were transfected, treated with 0.5 UM tamoxifen for 24 hours at 1 day post-transfection, and collected samples at days 3, 5 and 7 and performed amplicon sequencing of shuffle cassette barcodes. Both inducible Cre variants induced far fewer SVs than constitutive Cre, and also failed to facilitate the survival of cells bearing SVs at day 7. As an alternative strategy, cell death was prevented by treating cells with the p53 inhibitor Pifithrin-α (20 μM) for 48 hours post-Cre transfection (but not beyond that due to the toxicity and effects on stem cell maintenance and differentiation of p53 inhibition (47,48)). However, although Pifithrin-α treatment increased the number of detected SVs at day 3, a sharp decline in the abundance of SVs was observed by day 5.

An alternative explanation is that the Cre-induced SVs are causing fitness defects, such that cells with fewer phenotypically consequential SVs, or those lacking SVs entirely, outcompete them in the population. If this were the case, sorting out rearranged cells into single wells such that they can grow in isolation would be expected to solve this problem. Either Cre or Bxb1 recombinase was co-transfected, together with a Cre-reporter that conditionally expresses red fluorescent protein (RFP), into the bottlenecked parental population. Transfected cells were either treated with Pifithrin-α or no drug for 48 hours post-transfection. On day 3, 720 RFP-positive cells from the Cre condition samples were sorted into single wells of 96-well plates that either contained Pifithrin-α or no drug. Pifithrin-α treatment led to a marked increase in the number of clones that grew up after single-cell sorting from the Cre-transfected population, consistent with p53 inhibition reducing cell death. However, no SV-supporting barcode combinations were detected upon sequencing barcode pairs in genomic DNA derived from 86 single-cell clones. Interestingly, the median number of parental shuffle cassettes detected per Cre-treated sample was lower than for Bxb1 samples. This suggests that clones with higher numbers of integrated shuffle cassettes may be selected against after Cre transfection.

Genome-shuffle-seq with Bxb1 recombinase in human cancer cells. The rapid depletion of Cre recombinase-induced SVs precludes the emergence of subclones for the functional analysis of specific SVs or SV combinations. To overcome this, Cre recombinase and/or p53-mediated apoptosis was avoided altogether. Specifically, a version of Genome-shuffle-seq relying on Bxb1 rather than Cre recombinase was developed, as well as expanding the evaluation to include human K562 cells, which derive from a p53-null, chronic myelogenous leukemia.

Bxb1 recombinase, which is known to exhibit lower toxicity than Cre recombinase in mammalian cells (49,50), utilizes two heterotypic recombinase sites, attB and attP. Recombination between these sites results in the formation of novel attL and attR sites, which are resistant to further Bxb1-mediated recombination. As such, SVs resulting from Bxb1-mediated recombination of attB/attP sites are expected to be more stable than those resulting from Cre-mediated recombination of IoxPsym sites. Although the use of heterotypic sites means that only 50% of possible pairs of integrated shuffle cassettes will be able to recombine with one another, this is balanced by an advantage with respect to detection rate. In particular, the directional nature of these Bxb1-targeted sites ensures that recombined shuffle cassettes will be flanked by heterotypic primer binding sites, which eliminates the aforementioned concern about suppression PCR (41, 42) and thus increases the likelihood that the Genome-shuffle-seq-induced rearrangements will be detectable (FIGS. 2C, 2D, 8A, and 8B).

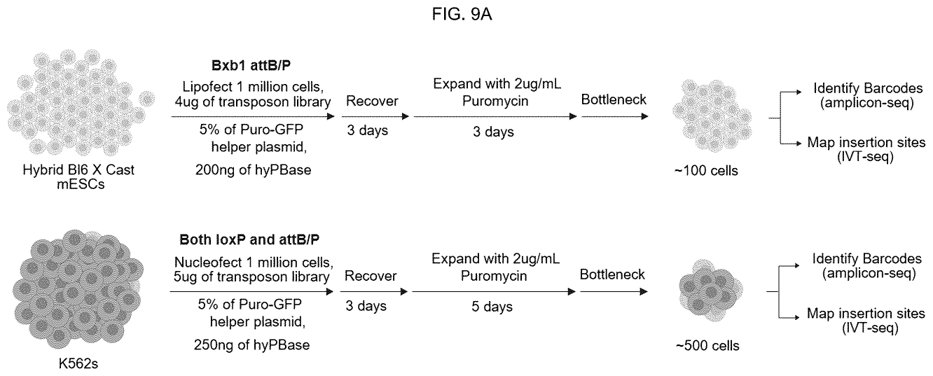

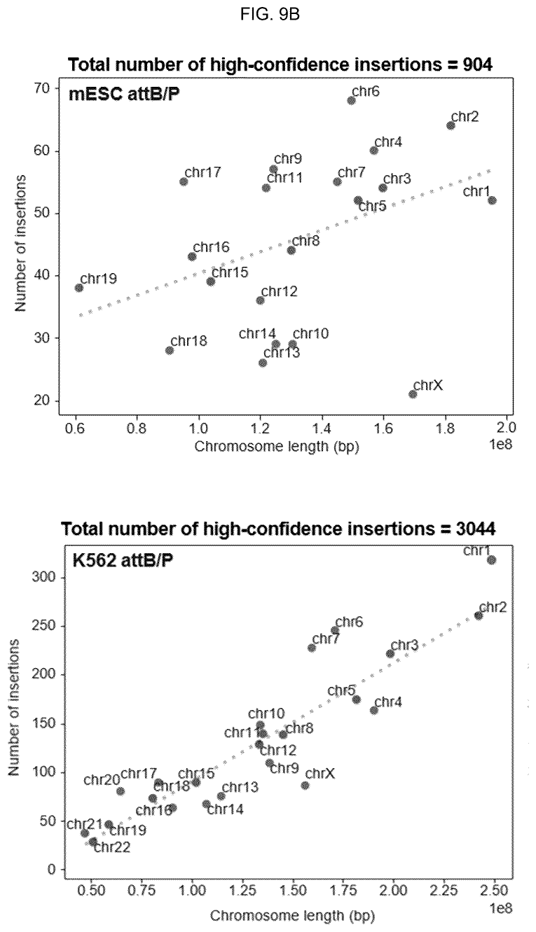

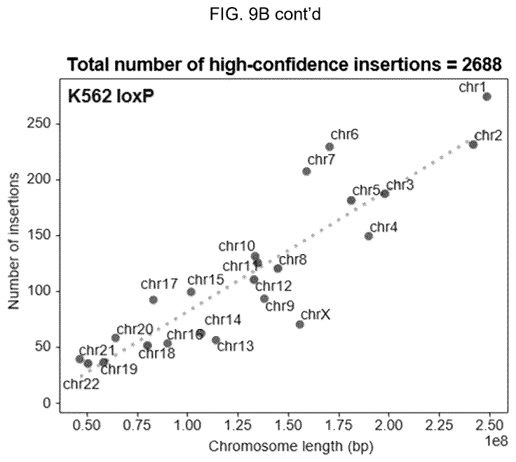

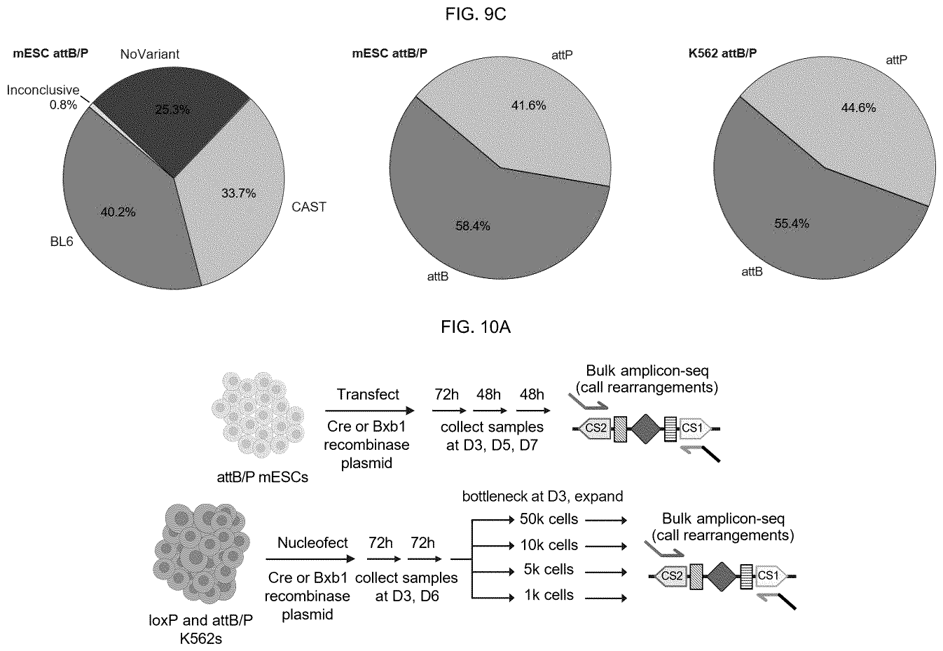

A Bxb1 attB/P shuffle cassette library was introduced to both mESCs and K562s, and in parallel, a IoxPsym shuffle cassette library was introduced into K562s, at a high MOI (FIG. 9A). As previously, bottlenecked founder populations were expanded, and insertion sites were mapped with IVT-seq (37). The precise genomic locations of 904, 3644 and 2688 shuffle cassettes in attB/P+mESCs, attB/P+K562s and IoxPsym+K562s, respectively (FIG. 9B) were confidently mapped. 74% of mapped mESC attB/attP cassettes were confidently assigned to either the BL6 or CAST allele, and mapped attB vs. attP cassettes were present in roughly equal proportions (FIG. 9C).

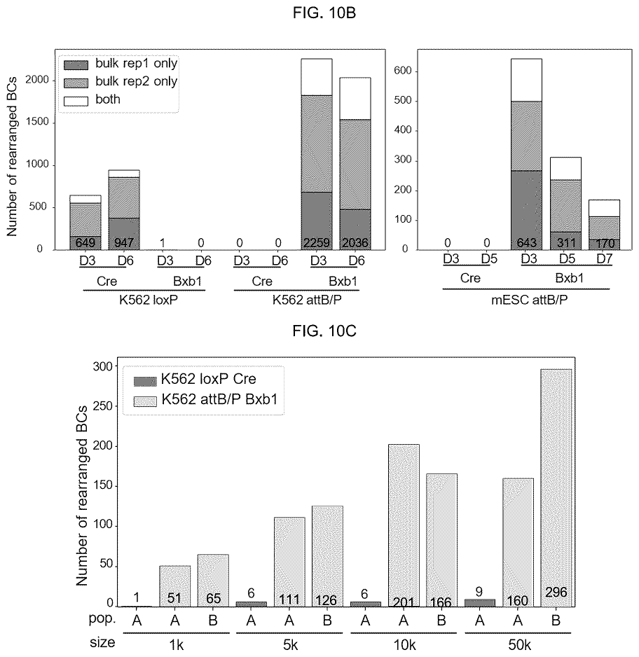

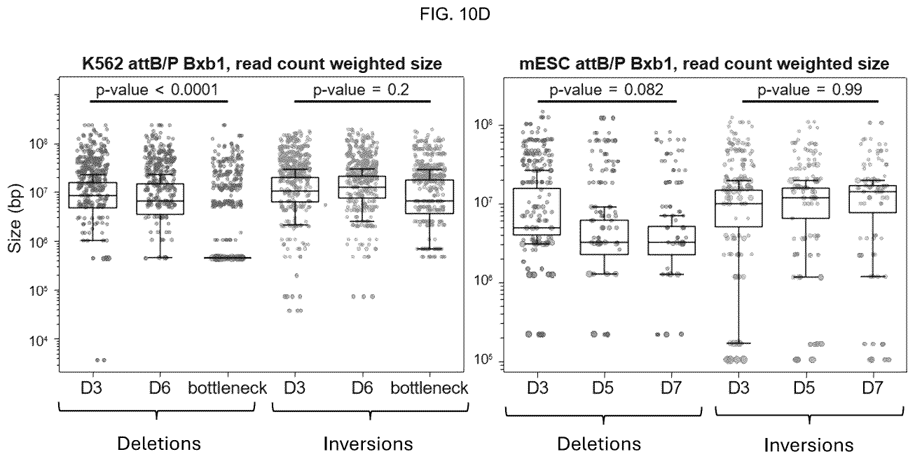

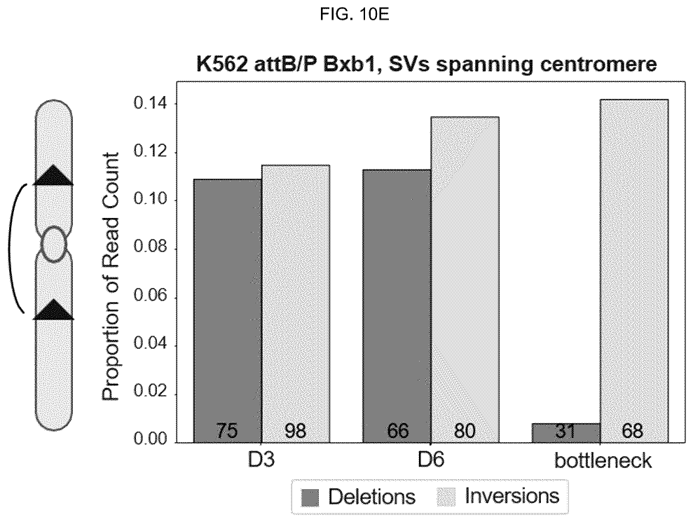

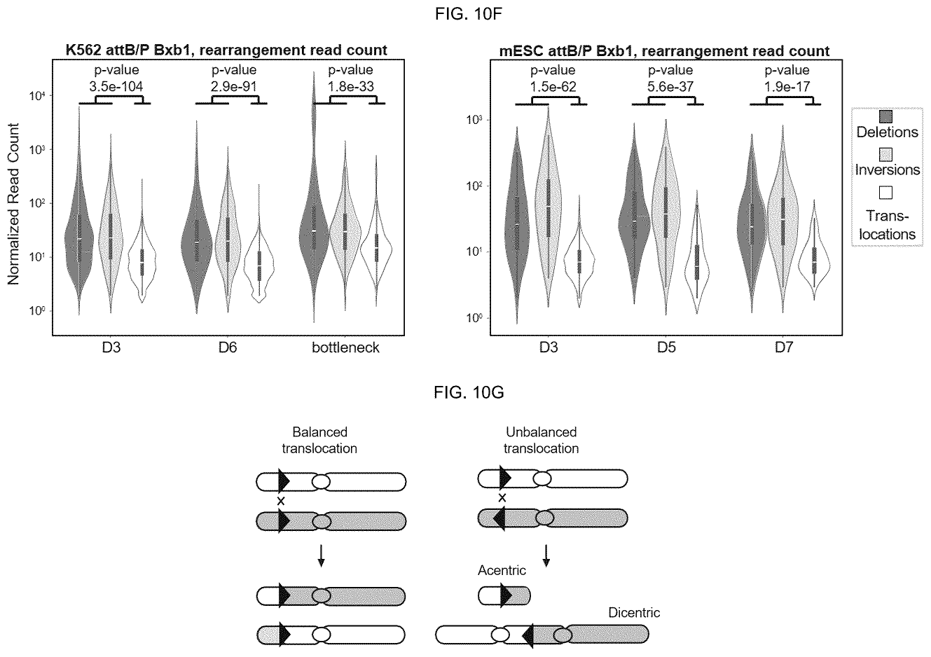

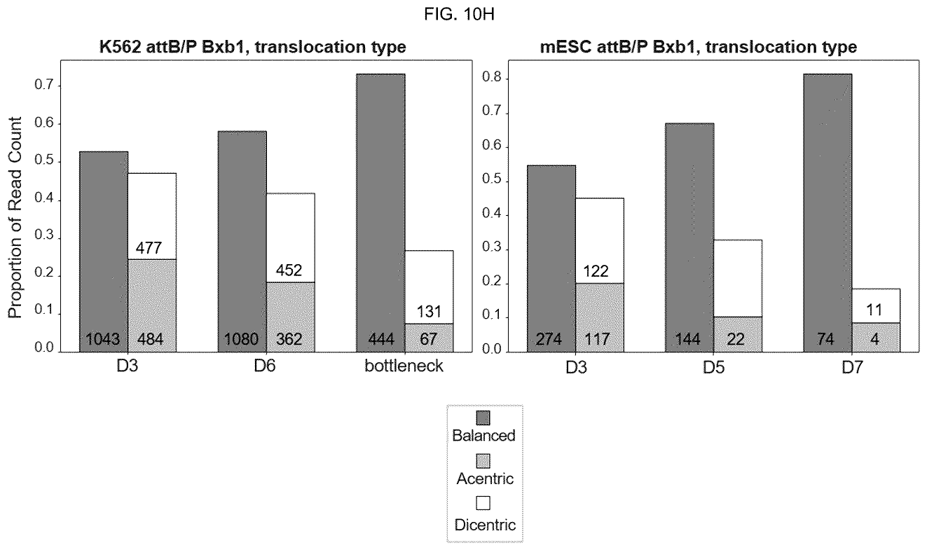

Once these lines were established, they were transiently transfected with Cre or Bxb1 recombinase expressing plasmids (FIG. 10A). For mESCs, cells were sampled at days 3, 5 and 7 post-transfection. For K562s, cells were sampled at days 3 and 6 post-transfection. In addition, Bxb1-treated attB/P+K562s were bottlenecked at day 3 to 50,000, 10,000, 5,000 and 1,000 cells and harvested 2 independent populations per bottleneck size after expansion (>8 days from treatment). Cre-treated IoxPsym+K562s were bottlenecked at day 3 but harvested 1 population per bottleneck size after expansion (>8 days from treatment). Genomic DNA was extracted from the samples, and amplicon sequencing of shuffle cassettes was performed to detect barcode combinations present in these samples.

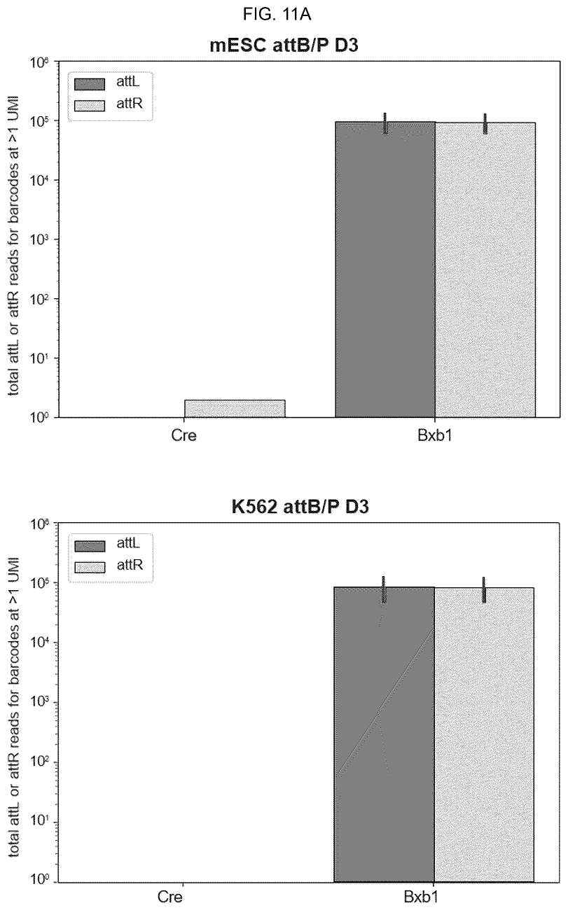

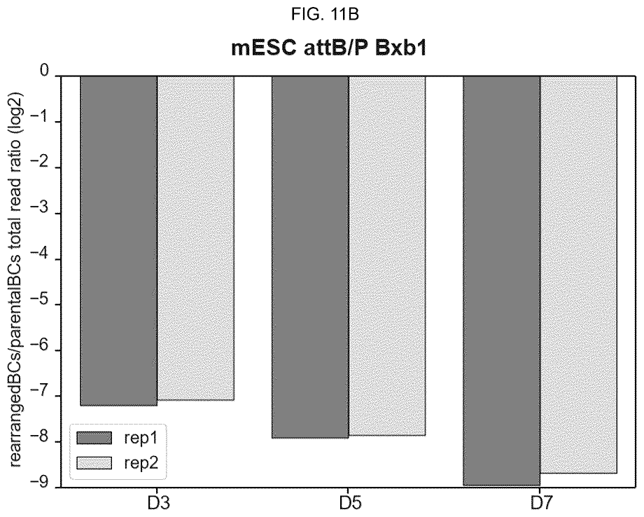

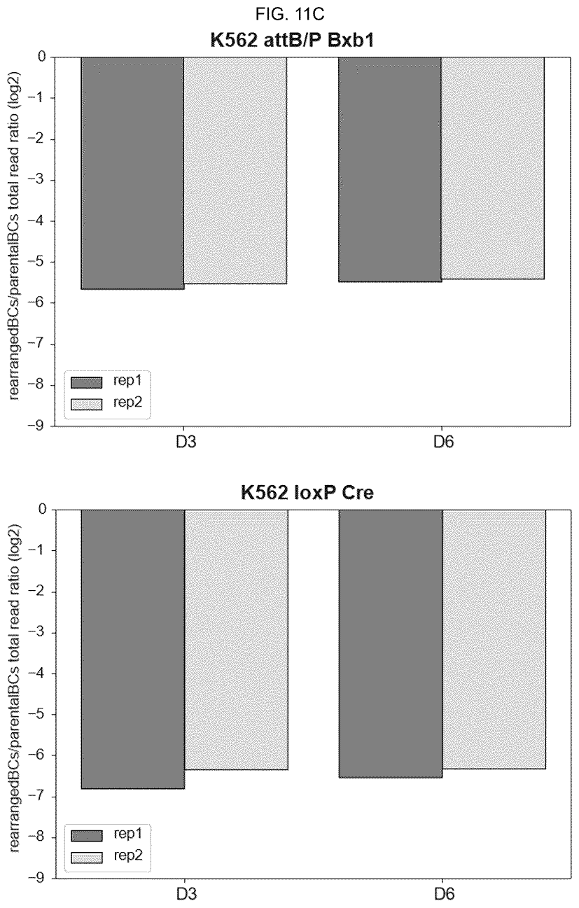

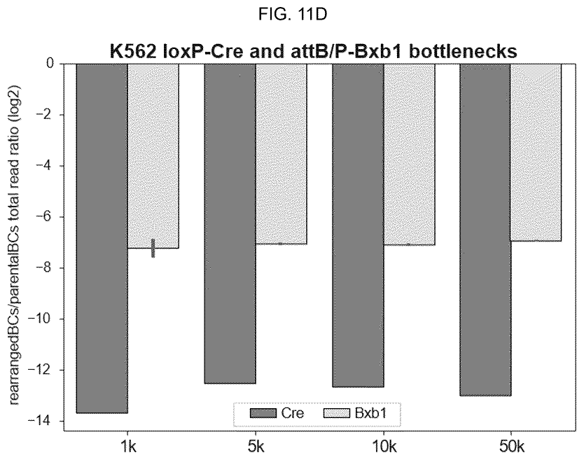

Hundreds to thousands of rearranged barcode combinations were detected in cell lines transfected with a targeting recombinase, with no background in controls transfected with a non-targeting recombinase (FIG. 10B). As expected, in both attB/P+ cell lines transfected with Bxb1, rearranged barcode combinations now flanked newly formed attL or attR sites, rather than attB or attP sites (FIG. 11A). Furthermore, 100% of novel barcode combinations in K562s (n=6394) and mESCs (n=1399) across the samples could be matched to pairs of attB and attP sites detected in the original population. Overall, these results highlight the generalizability of Genome-shuffle-seq to diverse mammalian cell lines and site-specific recombinase systems, and further underscore its sensitivity and specificity.

SVs mediated by Bxb1 at asymmetric recognition sites are tolerated and survive bottlenecking. In contrast with their complete depletion after several days in Cre-treated IoxPsym+mESCs, rearranged barcodes continued to be detected in Bxb1-treated attB/P+mESCs at day 7 (compare FIG. 10C to FIG. 10B). Consistent with this, the ratio of amplicon reads bearing rearranged vs. parental barcode combinations decreased by >99.99% in Cre-treated IoxPsym+mESCs, but only 75% by day 7 in Bxb1-treated attB/P+mESCs. These results indicate that rearrangements induced by Bxb1 at asymmetric recognition sites are much better tolerated than those induced by Cre at symmetric recognition sites, at least in mESCs.

In human K562 cells transfected with a targeting recombinase (i.e., both Cre-treated IoxPsym+K562s and Bxb1-treated attB/P+K562s), the number of rearranged barcode combinations as well as the ratio of rearranged vs. parental barcode combinations remained relatively stable at days 3 and 6 (FIGS. 10B and 11C). Furthermore, Bxb1 appeared more effective at inducing SVs than Cre (FIG. 11C), potentially reflecting its greater efficiency in mammalian cells (51).

To summarize the contrast between the cell lines, both Cre- and Bxb1-mediated SVs persisted for over 6 days in K562 cells, while in mESCs, SVs were either completely (Cre) or partially (Bxb1) depleted within a week (FIGS. 10B, 10C-10D, and 11B-11C). Possible explanations for this difference include: 1) p53-null K562 cells are less sensitive to recombinase-induced genotoxicity; 2) K562 cells divide more slowly than mESCs, such that the recombinase-expressing plasmid may still be present and inducing new rearrangements at later time points; and/or 3) K562 cells are grown in suspension, which increases the chance of dead/dying cells to contaminate the sample, in contrast with adherent mESCs with which unhealthy cells are lost in the supernatant.

To distinguish between these possibilities, the number of rearrangements were examined in the recombinase-treated K562 populations which had undergone bottlenecking and expansion (FIG. 10A). Here, the contrast between recombinases was stark, possibly because these conditions are the least prone to contamination by dead/dying cells. Whereas hundreds of SVs were readily detected in Bxb1-treated attB/P+K562s following bottlenecking and expansion, Cre-treated IoxPsym+exhibited a dramatic reduction in the number of surviving rearrangements (FIGS. 10C and 11D).

Overall, these results indicate that Cre and/or the rearrangements that it induces at symmetric recognition sites are toxic not only to mESCs but also to K562 cells. In contrast, Bxb1 and the rearrangements that it induces at asymmetric recognition sites are markedly better tolerated and more stable in at least two mammalian cell lines.

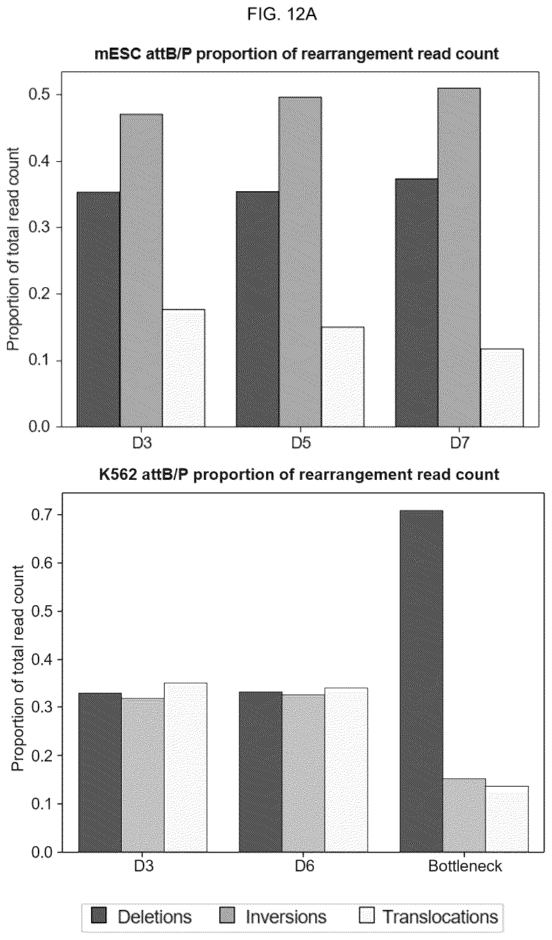

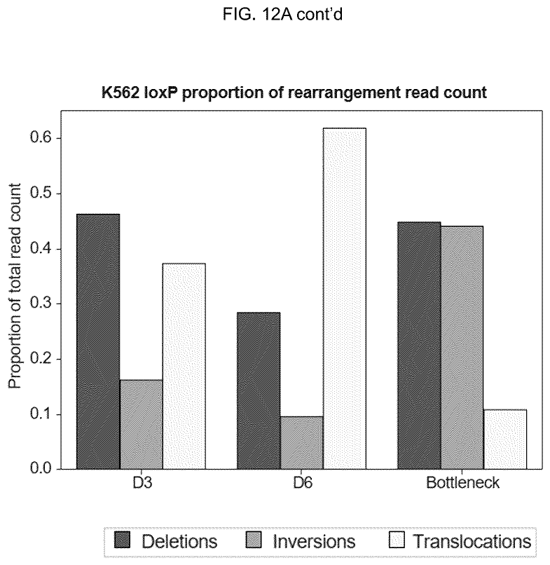

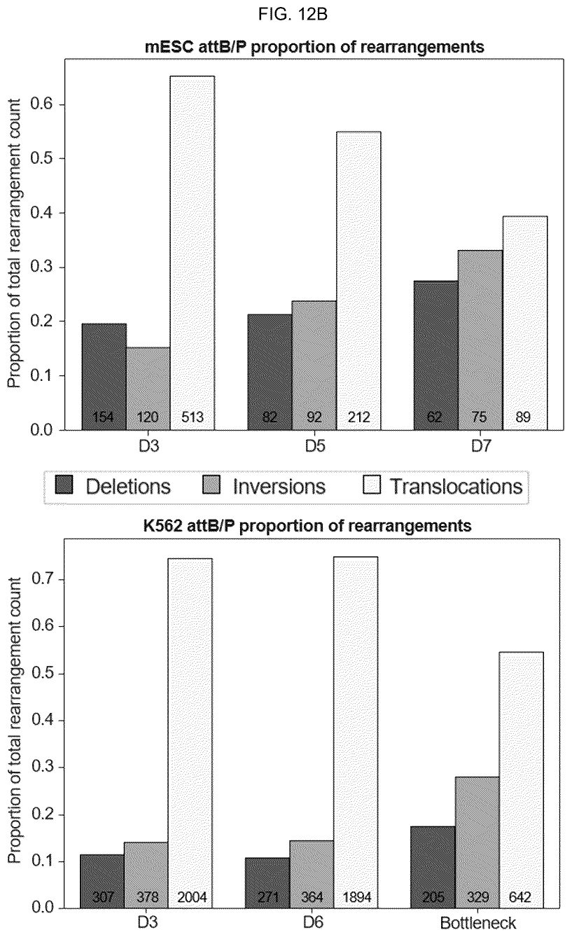

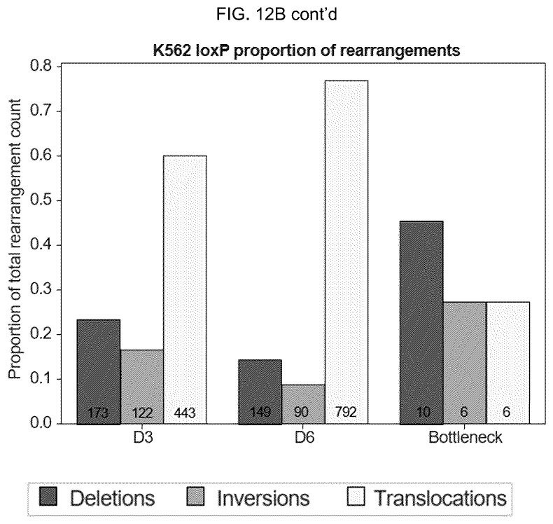

Bxb1 Genome-shuffle-seq mediated SVs exhibit signatures of selection. As these are relatively short culture-based experiments in diploid (mESC) or pseudotriploid (K562) cells, most induced SVs are not expected to exhibit strong signatures of selection. Nonetheless, the data were examined to evaluate whether any particular class of SVs was rapidly enriched or depleted. As noted above, deletions, inversions, and translocations were detected in both Bxb1-treated attB/P+K562s and mESCs (FIGS. 12A, 12B). Focusing first on deletions and inversions, a clear inverse correlation was observed between SV event size and abundance, as expected due to the dependence of shuffle cassette recombination on proximity. The abundance-weighted size distribution of deletions, but not inversions, reduced over time and/or with bottlenecking (FIG. 10D). Further examination suggested that at least some portion of this effect is attributable to centromere-spanning deletions, which presumably compromise chromosome segregation. In particular, whereas centromere-spanning deletions are strongly depleted from bottlenecked K562 populations, centromere-spanning inversions are, if anything, enriched (FIG. 10E).

As with Cre-treated IoxPsym+mESCs, more unique translocations were observed in Bxb1-treated attB/P+K562s and mESCs than either deletions or inversions, but the underlying read counts once again revealed translocations to be much less abundant (FIG. 10F). Moreover, the diversity and abundance of translocations diminished over time and/or with bottlenecking (FIGS. 12A and 12B). To further investigate this, individual translocation SVs were classified as: 1) balanced; 2) unbalanced and resulting in an acentric chromosome; or 3) unbalanced and resulting in a dicentric chromosome (FIG. 10G). Intriguingly, while both balanced and unbalanced translocations are induced at equal frequencies, the proportion of unbalanced translocations of both subtypes decreased over time in both K562 and mESC populations (FIG. 10H). Furthermore, acentric chromosomes were depleted more rapidly than dicentric chromosomes (FIG. 10H), probably because dicentric chromosomes can survive a centromere crisis, whereas acentric chromosomes, lacking a centromere, cannot (52).

Taken together, these results show that cells bearing Bxb1-mediated SVs survive long enough to experience fitness effects caused by the SV rather than the recombinase, and highlight the potential for selective pressures on individual SVs generated by Genome-Shuffle-seq to be quantified.

Hundreds of ecDNAs are launched and detected by Genome-Shuffle-seq. Each Bxb1 recombinase-mediated intrachromosomal deletion between directly oriented sites is expected to leave a genomic scar composed of a shuffle cassette bearing a novel barcode combination, but also to create a single extrachromosomal DNA circle (ecDNA) composed of the deleted sequence and a shuffle cassette bearing the reciprocal barcode combination (FIG. 1A). Moreover, although both species are expected to be present in equal stoichiometry at the time of their formation, ecDNAs may be depleted over time due to their reliance on asymmetric segregation for inheritance. With Genome-Shuffle-seq, each novel, deletion-indicative barcode combination was assigned as derived from either a genomic scar or an ecDNA circle, based on the relative orientation of sites within the parental and recombined shuffle cassettes (FIG. 1E). Of note, recombination between shuffle cassettes on sister chromatids after genome replication could potentially yield duplications that are indistinguishable from ecDNAs based on amplicon sequencing (25). However, because the sites involved are in trans, such duplications are expected to arise at much lower frequencies than deletions, and are not considered for the analyses that follow.

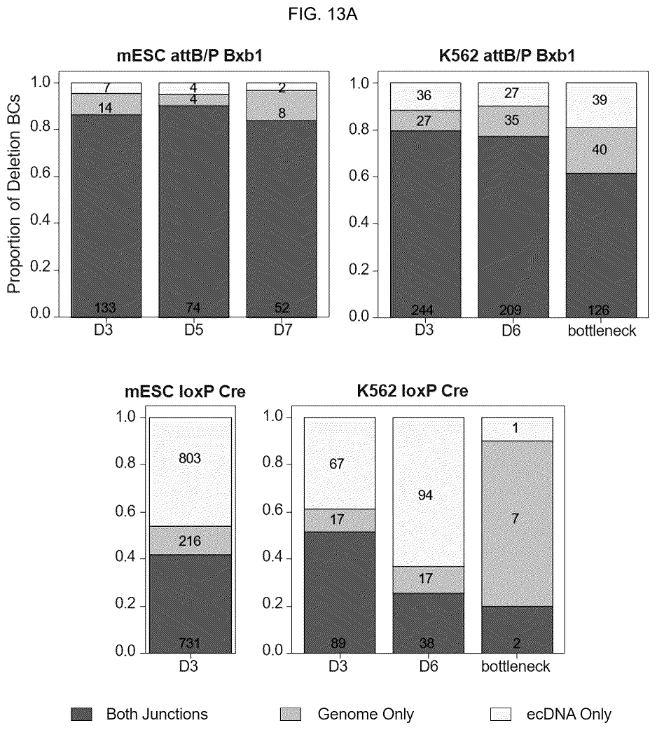

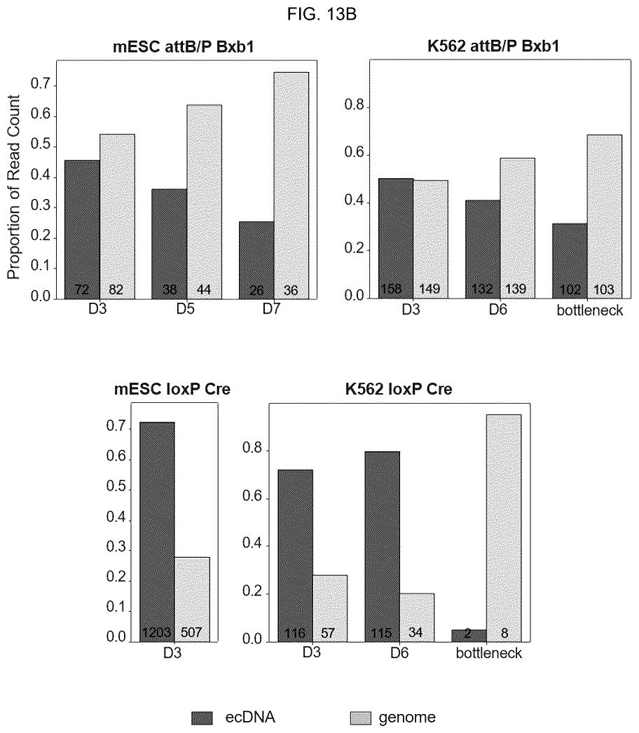

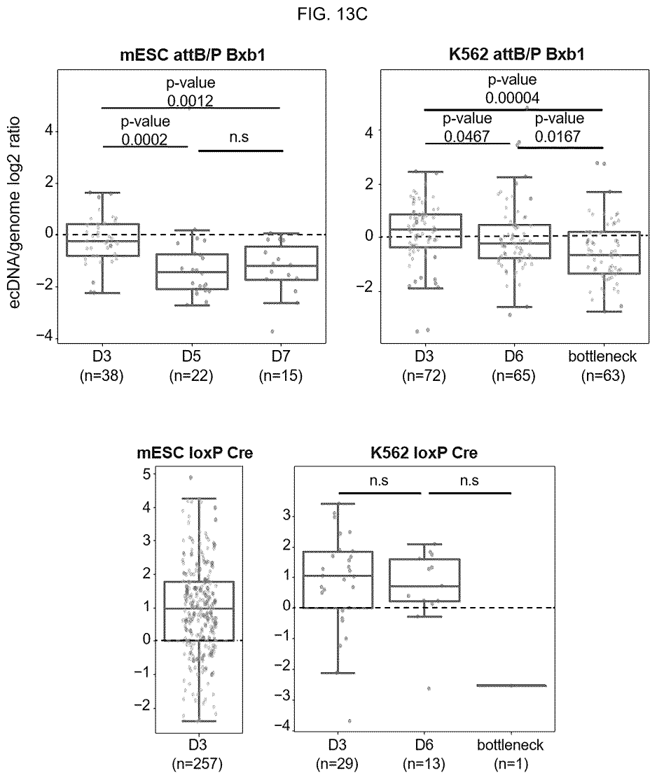

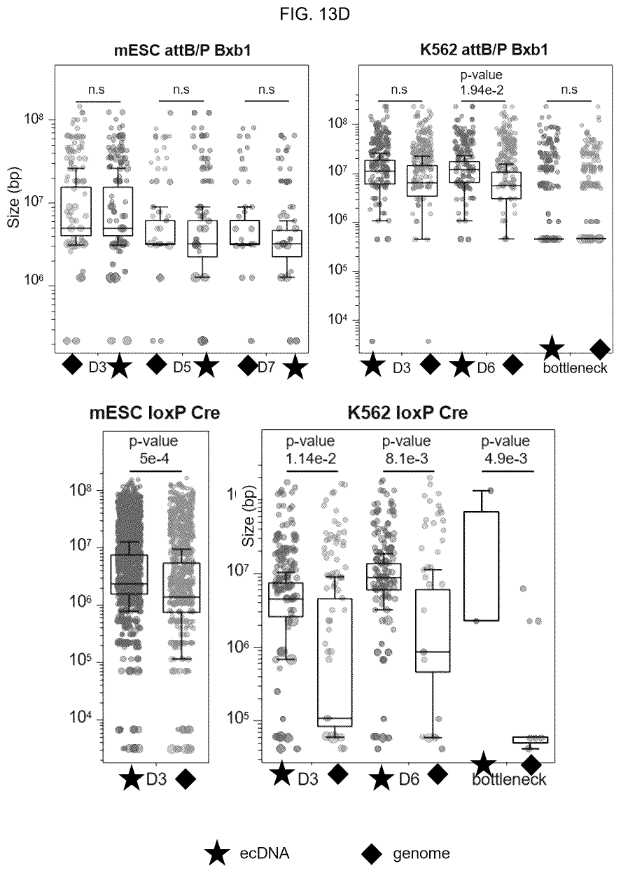

For the majority of deletions induced in Bxb1-treated attB/P+mESCs and K562s, reciprocal barcode combinations were readily detected derived from a “matched” genomic scar and ecDNA species in the same biological sample (FIG. 13A). Reciprocal barcode combinations were initially found at roughly equal frequencies, but as predicted, ecDNA barcode combinations were depleted over time, both overall as well as when only considering putative deletions for which both members of a reciprocal pair were detected (FIG. 13B-13D).

While results from Bxb1-mediated Genome-Shuffle-seq followed expectations for bona fide mammalian ecDNAs, results from the Cre-mediated experiments did not. First, there were lower proportions of cases in which both genomic scar and ecDNA-derived barcode combinations were detected in the same sample (FIG. 13A-13B). Second, in both Cre-treated IoxPsym+mESCs and K562s, barcodes derived from ecDNAs were detected at 2-fold higher read counts than barcodes derived from genomic scars, rather than the expected 1:1 stoichiometry (FIG. 13C). Finally, the inferred sizes of Cre-mediated ecDNAs tended to be larger than the inferred sizes of Cre-mediated deletion scars when weighted by abundance in the population (FIG. 13D).

The deviation of Cre-mediated events from the expected 1:1 stoichiometry is not easily explained by differential recovery of ecDNA vs. genomic DNA, fluctuations in ecDNA copy number, or the misattribution of some duplications to ecDNAs, as these features are shared between Bxb1- and Cre-derived ecDNAs. However, there were two differences: 1) symmetric sites (IoxPsym) were used with Cre, and asymmetric sites (attB/P) with Bxb1; and 2) post-recombination IoxP sites (including the symmetrical version used here) can undergo further recombination events, whereas the attL/R sites that result from Bxb1-mediated recombination of attB/P sites cannot. These differences may result in the formation of novel genomic structures in the Cre-based GenomeShuffle-seq experiments that are not easily decoded from shuffle cassette combinations. Additionally, given that very few Cre-mediated recombinants are detectable at later time points (FIGS. 10C and 13A), it is possible that some of these excess ecDNA barcode combinations originate from dead or dying cells, which may not accurately represent the distribution of induced SVs in (still) living cells.

In sum, these results indicate that Bxb1-mediated Genome-shuffle-seq may be a powerful tool to generate and study hundreds of ecDNAs launched from deletions throughout the genome.

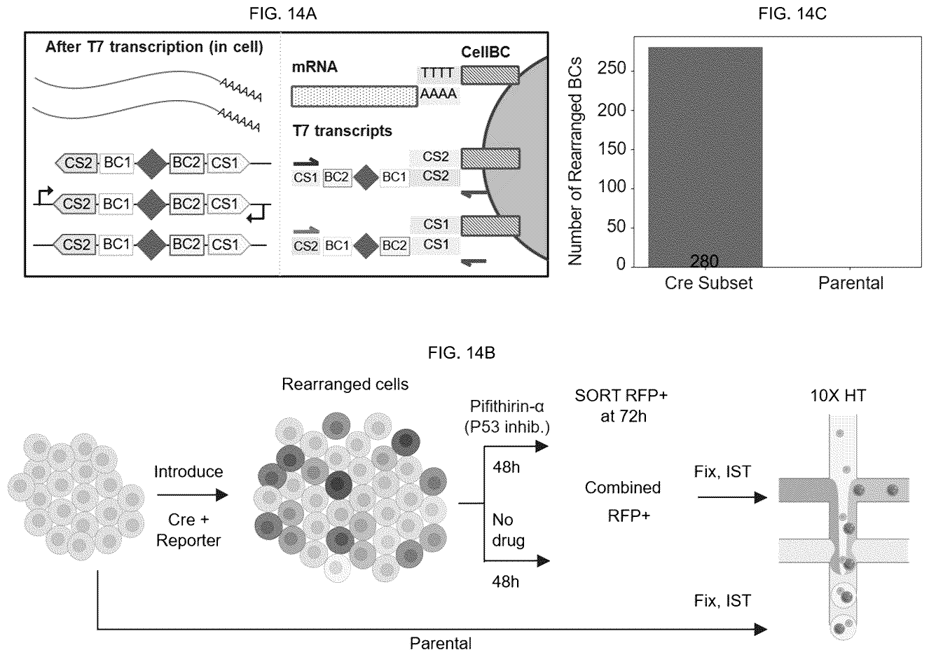

A co-assay of Genome-shuffle-seq-induced structural variants and single cell transcriptomes. Genome-shuffle-seq was designed such that the identity of induced SVs could potentially be co-assayed at single cell resolution with widely available scRNA-seq platforms (FIG. 1E). Specifically, after fixation and T7 IST (36,37), cells are expected to contain both endogenous mRNAs and T7-derived transcripts that span shuffle cassette barcode pairs. On the 10× Genomics platform, it should be possible to capture both sets of transcripts to a common cell barcode (cell BC) via 3′ scRNA-seq with feature barcoding (FIG. 14A).

As an initial test of this scheme, IoxPsym+mESCs were co-transfected with a plasmid expressing Cre recombinase together with a Cre-reporter that conditionally expresses RFP, sorted RFP+ cells at 72 hours, and then performed methanol fixation, T7 IST, and scRNA-seq. For this experiment, cells treated with and without Pifithrin-α were combined, and an independent sample from parental cells was also included as a control (FIG. 14B). 15,000 and 19,000 (T7 IST+scRNA-seq) profiles were recovered from Cre-treated and parental samples, respectively. To assess rearrangements, the shuffle cassette barcode combinations observed in (T7 IST+scRNA-seq) data were compared to parental barcode pairs, which identified 1123 novel barcode combinations. Given that Cre-treated samples were sequenced more deeply, novel barcode combinations were investigated to confirm that they not be an artifact of library construction by downsampling Cre-treated and parental data to similar sequencing depths. At similar depths, 280 novel barcode combinations were detected in Cre-treated scRNA-seq profiles, while 0 novel barcode combinations were detected in parental scRNA-seq profiles (FIG. 14C).

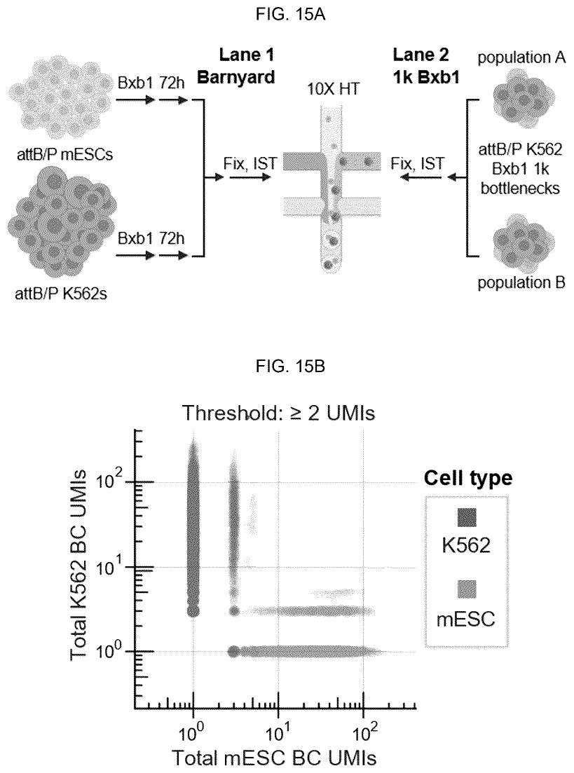

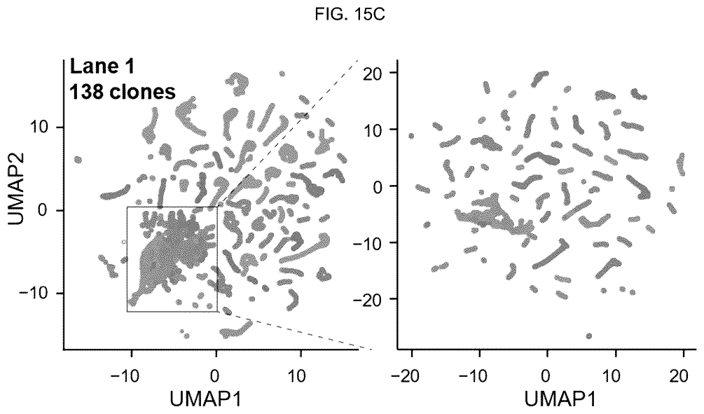

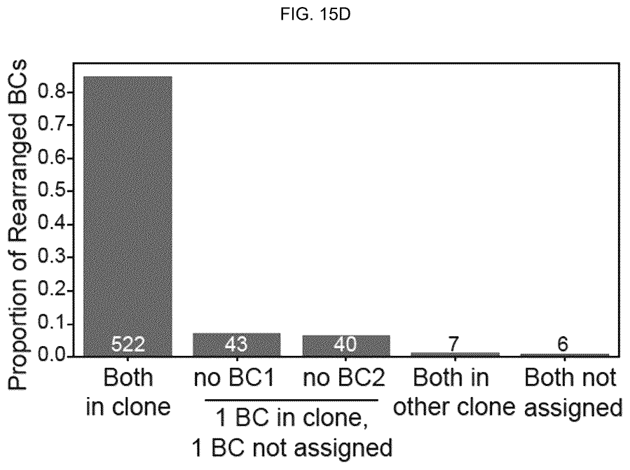

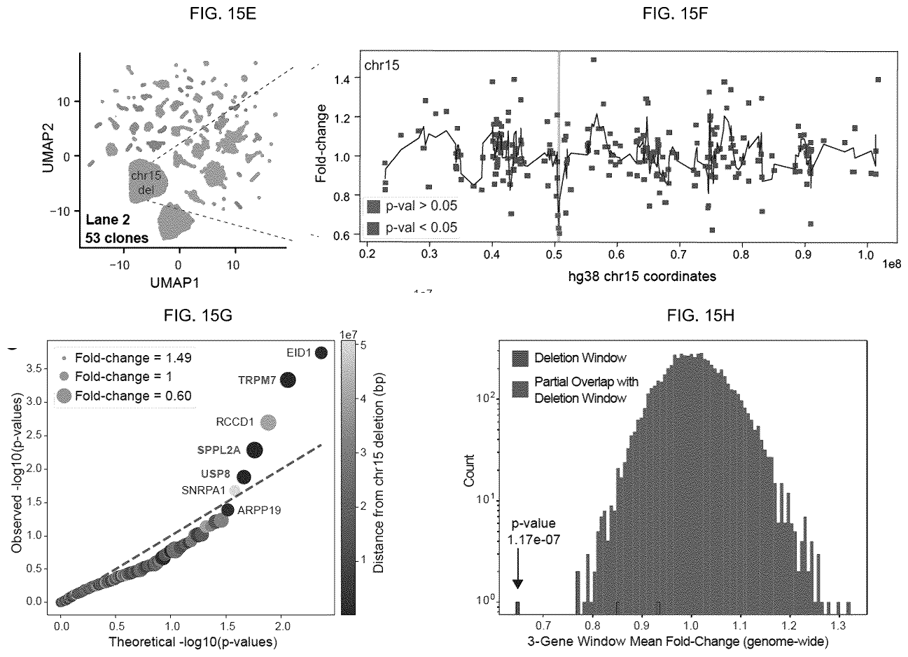

This preliminary experiment suggested that the single-cell co-assay was working as intended. However, two aspects triggered further investigation. First, because cells were permeabilized for T7 IST, the protocol may be more susceptible to ambient RNA contamination, a pervasive issue in scRNA-seq (55, 56). Second, co-capturing SV identity should allow testing for gene expression changes caused by induced rearrangements. To explore these aspects, we performed a second Genome-shuffle-seq scRNA-seq experiment (FIG. 15A). In a first tranche (“Lane 1”), a “barnyard” experiment was performed by mixing Bxb1-treated attB/P+mESCs (mouse) and K562s (human), prior to fixation and IST. In a second tranche (“Lane 2”), two independent populations of Bxb1-treated attB/P+K562s that had previously been bottlenecked to 1,000 cells were mixed, then expanded and subjected to amplicon-seq (FIG. 10C). In theory, cells with SVs passing the bottleneck should be expanded in the profiled population, potentially providing sufficient power for detecting gene expression changes caused by individual SVs.

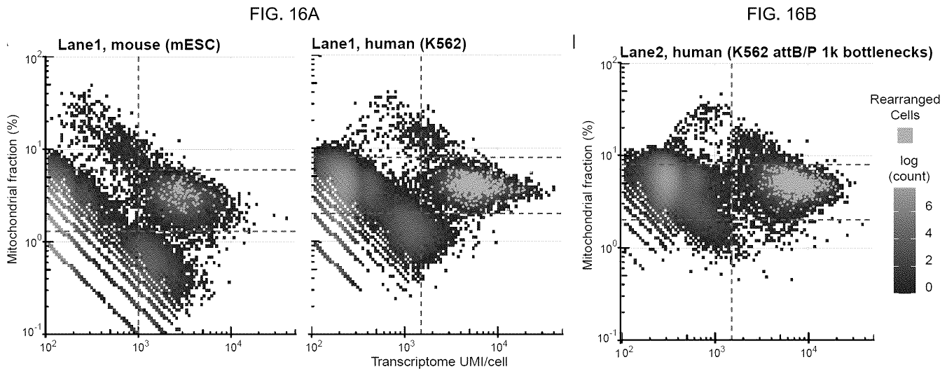

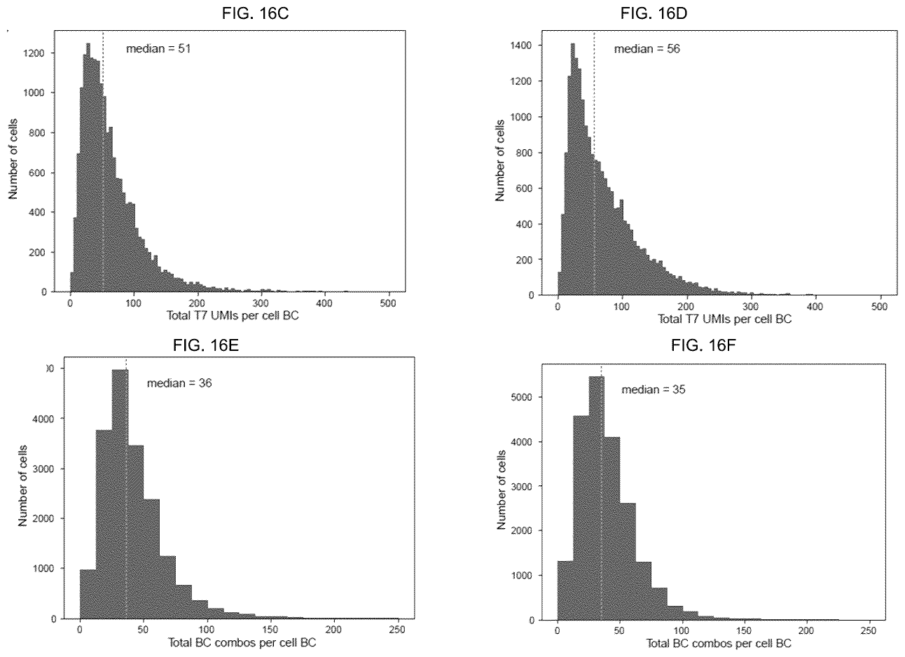

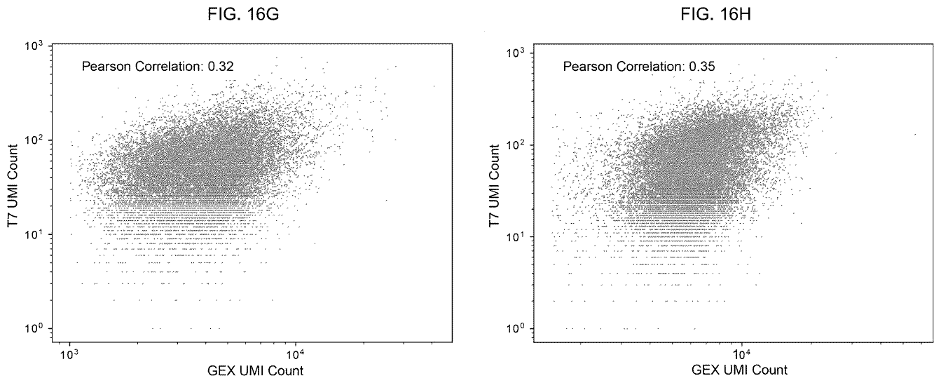

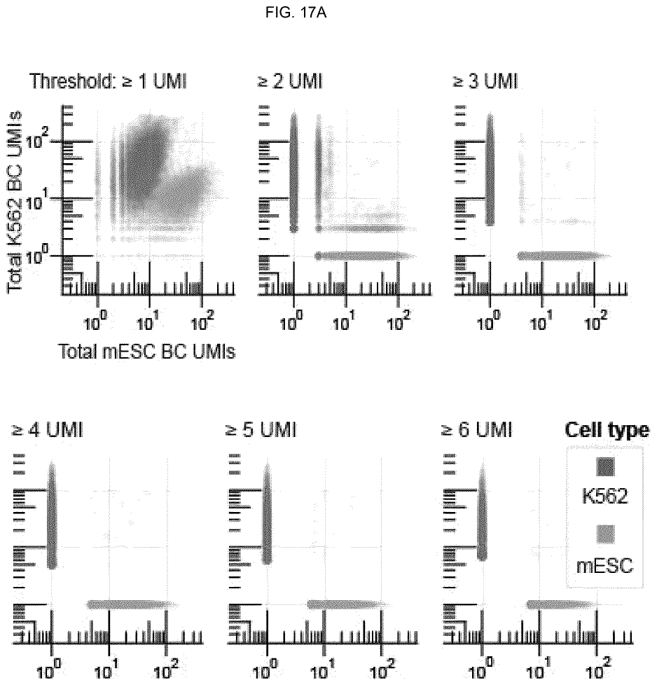

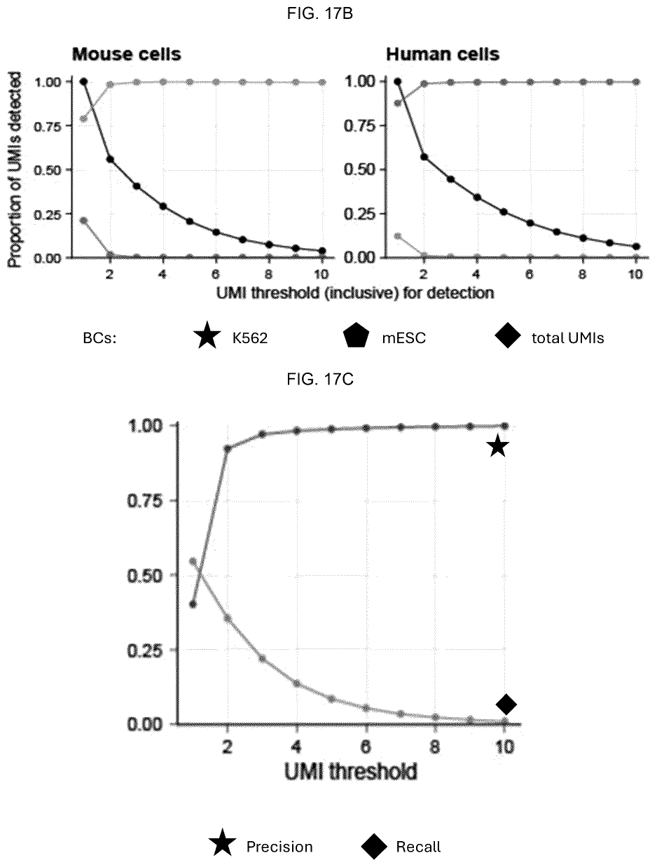

After filtering on mitochondrial content and transcriptome UMI counts, requiring detection of at least one T7 transcript, and performing doublet removal, 18,418 (11,113 K562 and 7,305 mESC) and 20,798 single cell profiles were recovered from the first and second tranches, respectively (FIGS. 16A-16B). A median of 51 and 56 T7 UMIs were also recovered, which reflected a median of 36 and 35 unique shuffle barcode combinations per cell (FIGS. 16C-16F). A median of 3,965 and 6,465 transcriptome UMIs were detected, and these counts were correlated with the number of T7 UMIs detected per cell (FIGS. 16G-16H). In the barnyard experiment, >75% of T7 BCs were associated with a single cell of the expected species, and a threshold of ≥2 UMIs per T7 barcode combination per cell was sufficient to achieve >98% species-specificity (FIGS. 15B, 17A, and 17B).

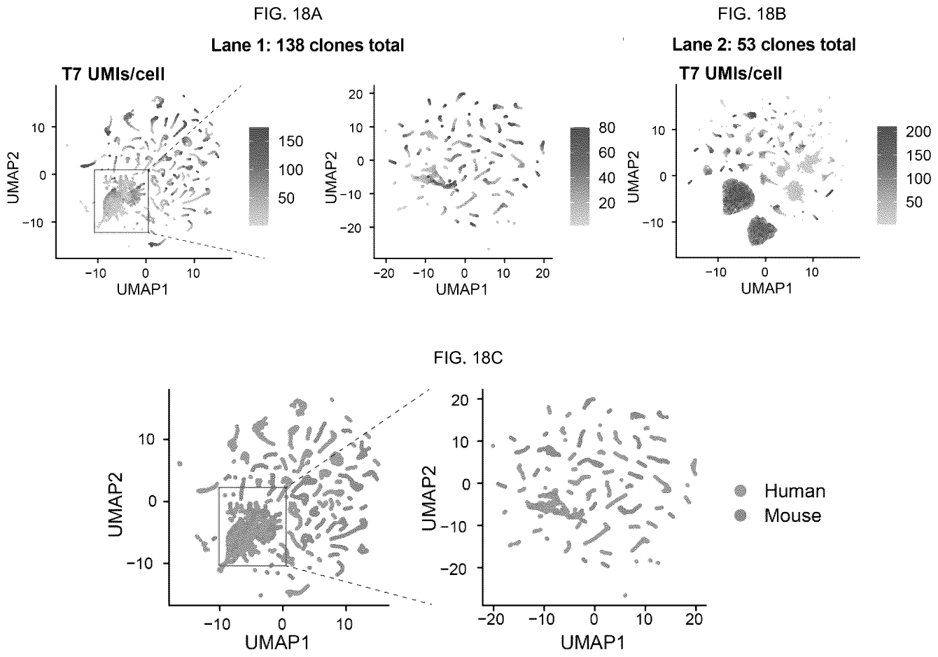

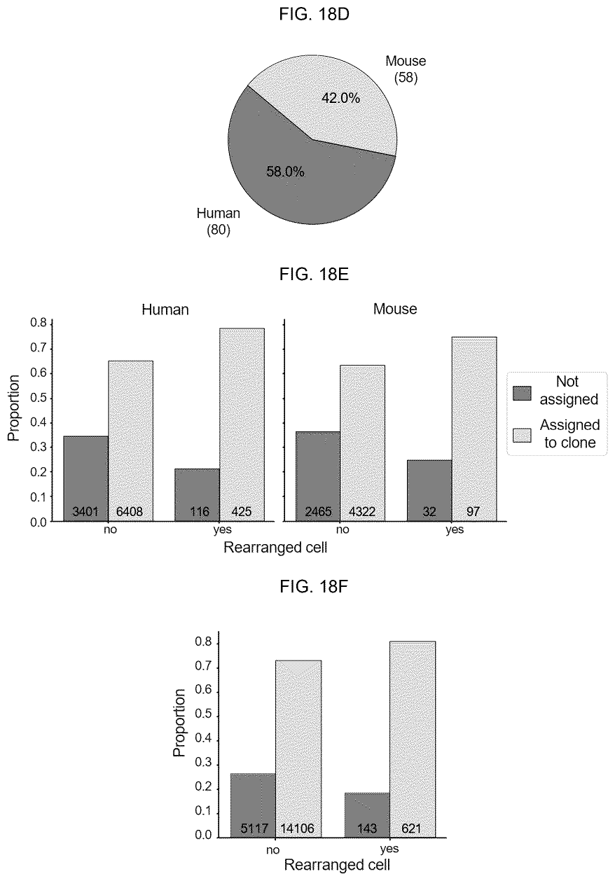

To further investigate the sensitivity of T7 barcode detection, iterative clustering was performed on the combinations of barcodes observed in single cells from the barnyard experiment, to yield “clonotypes”. It was anticipated that these clonotypes would correspond to individual cells that survived the original bottlenecking of the parental population (FIG. 9A), i.e. shuffle cassette barcodes assigned to a given clonotype correspond to the multiple piggyBAC integrations in a parental cell. Indeed, a more stringently filtered set of 11,252 cells clustered neatly in UMAP space based solely on their complement of T7 IST-derived barcode combinations into 138 species-coherent clonotypes (FIGS. 15C, 18A, and 18C-18E). Once again, a precision-recall analysis found that a threshold of ≥2 UMIs per T7 barcode combination per cell was sufficient to achieve high specificity, now with respect to clonotype rather than species (FIG. 17C).

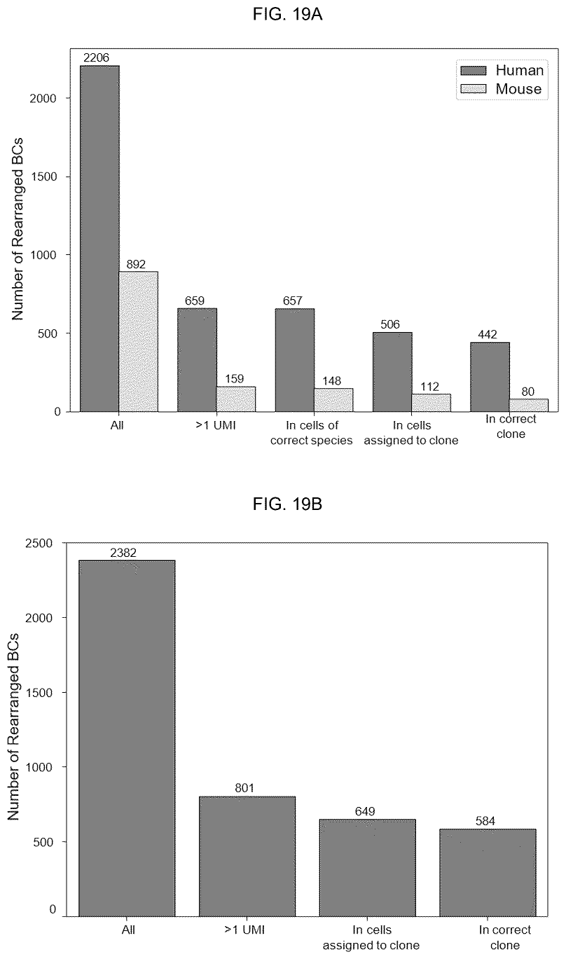

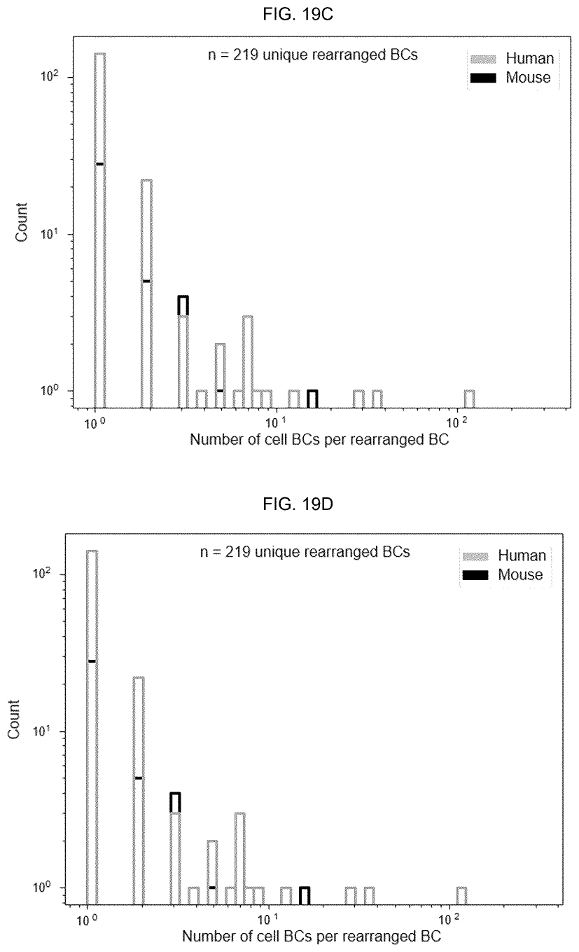

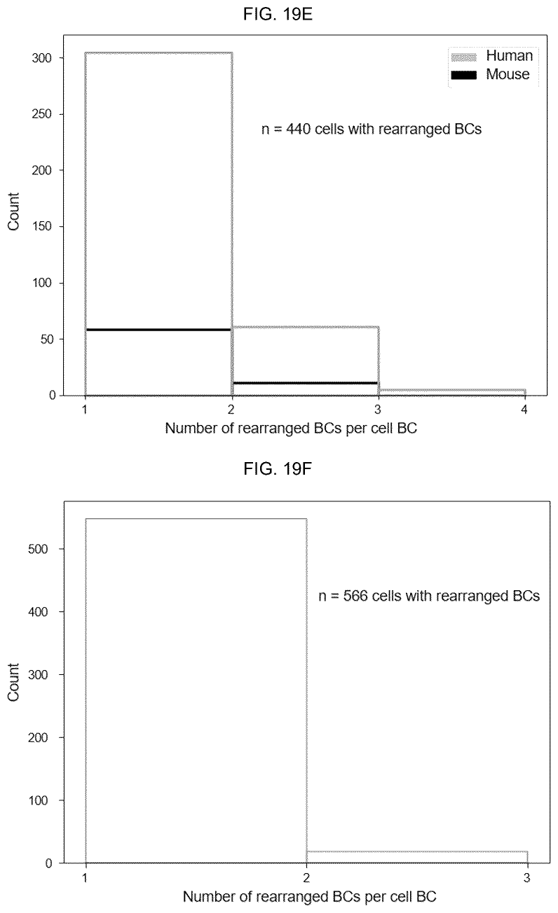

To assess rearrangements in the barnyard experiment, the shuffle cassette barcode combinations observed in (T7 IST+scRNA-seq) data were compared to parental barcode pairs. 3098 novel barcode combinations, 618 of which were found at >2 UMIs in cells of the correct species that were assigned to a clonotype (FIG. 19A). The barcodes contributing to these novel combinations were highly congruent with expectation based on clonotype identity. Specifically, 84% involved a pair of parental barcodes from the same clonotype, while 13% involved one parental barcode from the same clonotype, and one parental barcode that was unassigned to any clone (FIG. 15D). Collectively, these data suggest that it is possible to detect, map, and confidently assign induced SVs to single cells with associated transcriptomes.

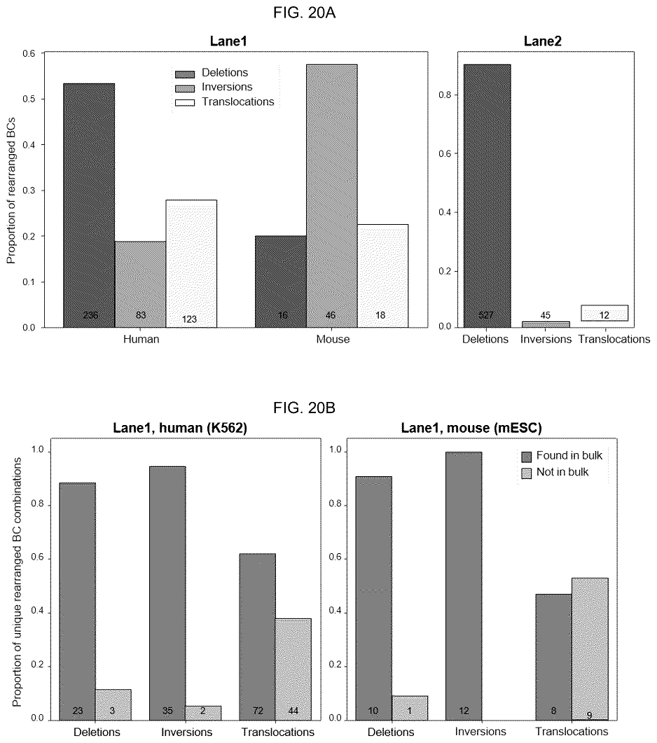

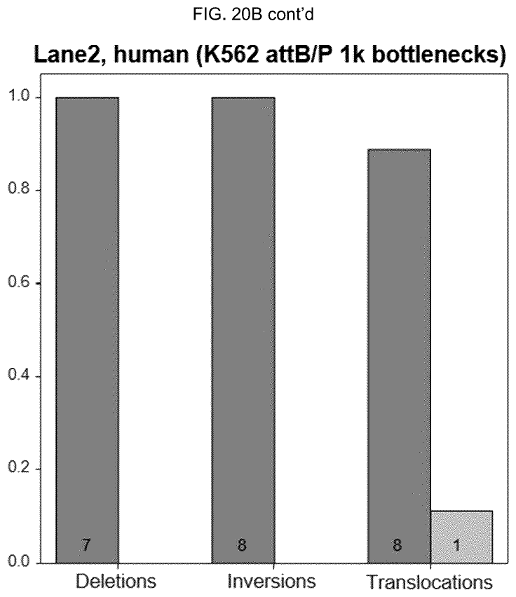

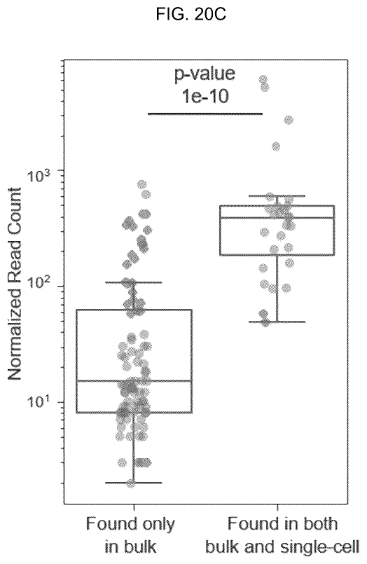

For Lane 1, most rearrangements were only detected in one cell (range 1-115), and most cells only contained a single detected rearranged BC pair (range 1-3) (FIGS. 19C-19E). Similar to the analysis shown in FIGS. 5A-5G, the nature of each of the SVs can be inferred based on the parental locations of the barcodes contributing to each novel pair, which once again included deletions, inversions and translocations (FIG. 20A). Intriguingly, even though bulk and single cell data derived from independent Bxb1 transfections of this parental population, the vast majority of deletions and inversions detected in single-cells were also detected in bulk amplicon-seq data, but a much smaller fraction of translocations were similarly replicated (FIG. 20B). This likely results from the fact that there are a combinatorially larger number of possible translocations than inversions or deletions, which makes it less likely for the same translocation to recur across samples.

A similar analysis was performed for data from the Lane 2 bottlenecking experiment, assigning 14,727 cells to 53 clonotypes (FIGS. 15E, 18B, and 18F). 584 rearranged barcodes were detected at >2 UMIs across 566 cells that were correctly assigned to a clone (FIG. 19B). These rearranged barcodes corresponded to 24 unique novel barcode combinations, of which 23 were detected in bulk amplicon-seq data from the same bottlenecked population. The inferred abundances of these 23 combinations were markedly higher than those of other novel combinations detected by bulk amplicon-seq (FIGS. 20B, 20C).

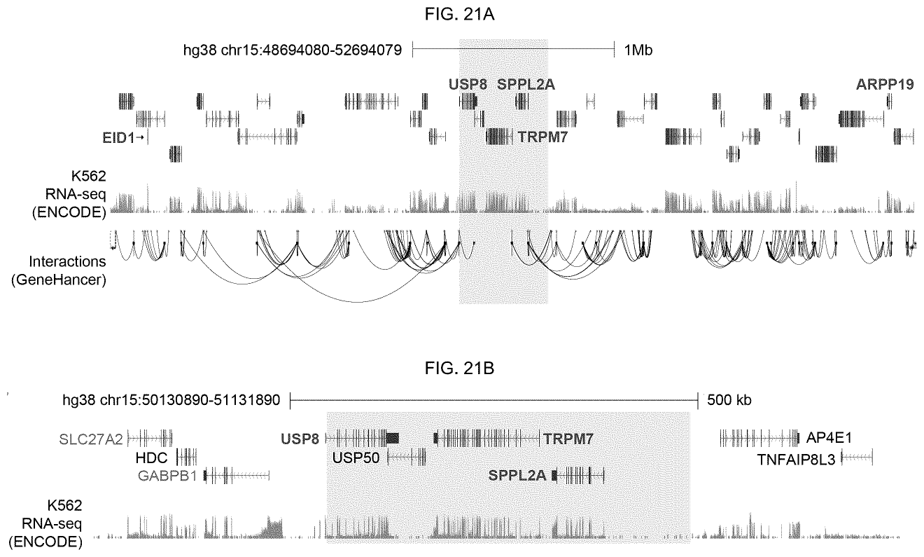

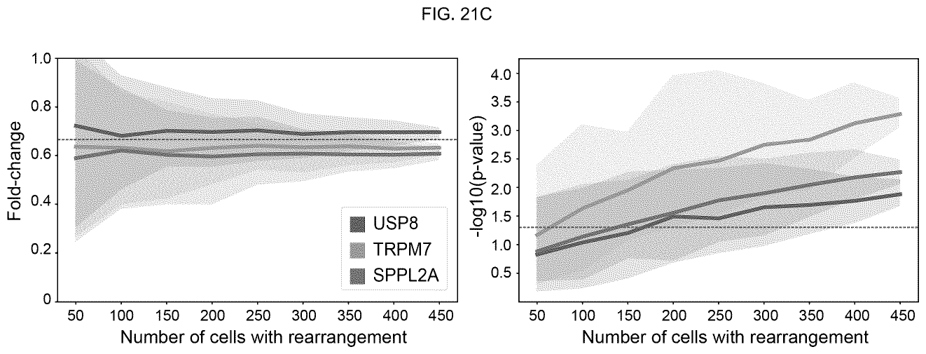

Despite the bottleneck, most of these 24 novel barcode combinations were detected in fewer than 10 cells (FIGS. 19D, 19F), precluding the sensitive detection of gene expression changes consequent to a given SV. However, there was one exception, a novel barcode combination indicative of the genomic scar of a 447 kb deletion on human chr15 that was detected at >2 UMIs in 465 cells that were assigned to the correct clonotype and had associated transcriptomes (FIGS. 21A, 21B). Three genes within this interval were detected in >10 cells in the dataset (USP8, TRPM7, SPPL2A). The expression of these genes was compared in cells in which the rearrangement was detected to 680 cells from the same clonotype in which the parental barcodes for this rearrangement were detected (FIG. 15E). Differential gene expression analysis revealed that each of these three expressed genes that resided within the deletion interval exhibited a decrease in expression of 33%, which precisely matches expectation for deletion of a single allele of a triploid chromosome such as chr15 in K562s (FIG. 15F). The differential expression of these three genes, when considered individually, were nominally significant, as were two other genes located 1.5 Mb away (EID1 and ARPP19) (FIGS. 15F, 15G). It is plausible that some long-range regulatory elements for the latter two genes lie within the deletion, although there was no obvious evidence for this in public datasets (FIG. 21A). The fact that these deviations are only nominally significant is presumably due to the limited number of cells available bearing this particular deletion. However, if the mean-fold change is considered of a rolling window of three genes, scanning throughout the genome, the trio of genes encompassed by the deletion is a clear, significant outlier (FIG. 15H).

To assess the robustness of this result, a downsampling analysis was performed, reducing the number of cells in which the novel barcode combination indicative of the 447 kb deletion was used in the comparison. Although statistical significance is unsurprisingly a function of the number of rearrangement-bearing cells available for the comparison, the estimate of a 33% reduction in expression for the three genes (USP8, TRPM7, SPPL2A) was highly stable to downsampling (FIG. 21C).

Taken together, these results demonstrate that Genome-shuffle-seq is compatible with scRNA-seq, and that co-assays of single cell transcriptomes and SV-informative barcode combinations can facilitate the quantification of gene expression changes resulting from induced SVs.

Discussion Here, Genome-Shuffle-seq, a novel method for the inducible generation and facile characterization and quantitation of thousands of mammalian SVs within a pool of cells, without the need for any clonal isolation/genotyping nor for any whole genome sequencing, is described. It is shown how Genome-Shuffle-seq can be used to produce synthetic ecDNAs in mammalian cells and to quantify selection acting on the landscape of induced SVs. Finally, it is demonstrated that SV identities can be captured alongside single-cell transcriptomes for at least hundreds of such rearrangements, and the potential for such data to reveal changes in gene expression caused by induced SVs.

Genome-Shuffle-seq lays the foundation for large-scale single-cell genotype-to-phenotype screens of the impact of thousands to millions of mammalian SVs and ecDNA species on gene expression, chromatin structure, and genome organization, analogous to Perturb-seq or CROP-seq (56,57). As a related approach, the targeted introduction of shuffle cassettes to individual mammalian genomic loci, for instance by bottom-up assembly (58), would facilitate the dissection of regulatory element interactions and locus architecture in specifying gene regulation. Genome-shuffle-seq could also readily be adapted to study the cell-type-specific impact of SVs by differentiating a single engineered population into in vitro multi-cellular models or in vivo using whole organism models. Of note, a complementary strategy for the “randomization” of mammalian genomes with engineered SVs can be done using highly multiplexed prime editing-mediated insertion of IoxPsym sites (59). Beyond enabling the more systematic study of SVs and ecDNAs, these approaches may also serve as an entry point for the engineering of a ‘minimal genome’ comprising the complement of genetic information utilized for propagation of any mammalian cell, potentially useful as a universal chassis for cell-based therapy (60).

Methods. Shuffle cassette library cloning. The sequence of the shuffle cassettes were ordered as single-stranded oligonucleotides from Integrated DNA Technologies (IDT) with degenerate bases at the appropriate sites to serve as barcodes (FIG. 1B). The variant Bxb1-GA attB and attP sites were employed, as they were previously shown to be more efficient than the canonical GT variant in mammalian cells (51). The pool was PCR amplified using Q5 polymerase (NEB M0492S) for 8 cycles with primers that contained overhangs for subsequent cloning. PCR products for IoxPsym library cloning were run on a polyacrylamide gel (PAGE) and the band at the appropriate size was excised and purified. Bxb1 attB/P library PCR products were cleaned up (1λ) with Ampure XP beads (Beckman A63882). Purified product was Gibson cloned into a previously described PiggyBac transposon vector (37) using standard protocols in a 10 μl reaction (NEB E2621S). 2.5 μl of the Gibson reaction was electroporated into 25 μl of 10-beta electrocompetent E. coli (NEB C3020K). The transformed library was grown overnight in 50 ml of liquid Luria Broth (LB)+50 μg/mL Ampicillin and purified using the ZymoPURE II Plasmid Midiprep kit (Zymo D4200) according to the manufacturer's instructions.

Cell culture and integration of Shuffle Cassette Library. BL6xCAST mESCs were cultured at 37° C. and 5% CO2 on standard tissue culture-treated plates coated with 0.1% gelatin in “80/20” medium as previously described (58). To integrate the Shuffle Cassette PiggyBac transposon library at high MOI, 4 μg of library DNA was reverse transfected with 200 ng of a PiggyBac Puro-GFP helper plasmid (63) and 200 ng of a plasmid expressing the hyperactive transposase hyPBase (64) using Lipofectamine 3000 (Thermo L3000001) into 1 million cells. 3 days post-transfection, cells were selected with 2 μg/mL Puromycin. Surviving cells were bottlenecked to 100 founders by dilution and expanded.

K562s were cultured at 37° C. and 5% CO₂ on standard tissue culture treated plates in RPMI medium supplemented with 10% FBS (Hyclone). To integrate the Shuffle Cassette PiggyBac transposon library at high MOI, 5 μg of library DNA was nucleofected with 250 ng of a PiggyBac Puro-GFP helper plasmid and 250 ng of a plasmid expressing the hyperactive transposase hyPBase using an Amaxa 4D nucleofector (Lonza, SF cell line kit, program FF-120). Cells were selected with 2 μg/ml Puromycin, starting 3 days post-nucleofection. Surviving cells were bottlenecked to 100 founders by dilution and expanded.

MOI estimation by qPCR. Genomic DNA was extracted from cells using the Qiagen DNeasy kit. MOI of the shuffle cassette (primers oSP990-oSP993) and Puro-GFP (primers oJBL043-oJBL044) were measured relative to two genomic targets Tfrc (primers oJBL0276-oJBL0277) and Tert (oJBL0280-oJBL0281), which are expected to have 2 copies in these cells (63). qPCR was performed using 2× PowerUp Sybr Master Mix (Thermo A25742) with 50 ng of genomic DNA as template per 10 μl reaction with 0.5 μl of 5 UM primer mix. qPCRs were run on a BioRad CFX Opus Real Time PCR instrument in triplicate. Cycle threshold values were averaged across triplicates, and copy number was estimated using the ΔCT method relative to each genomic target independently, corrected for the presence of 2 genomic copies, and then averaged across the two targets.

Recombinase plasmid transfection and cell population maintenance for shuffle experiments. Recombinase expression plasmids used in this study are pCAG-iCre (Addgene #89573), pCAG-CreERT2 (Addgene #14797), pCAG-ERT2CreERT2 (Addgene #13777) and pCAG-Bxb1 (pSP0722, modified from Addgene #51271).

For mESC experiments, between 200,000 and 350,000 cells were reverse transfected with the specific recombinase plasmid using Lipofectamine 3000 (Thermo L3000001) in 6-well plates with the exception of the data shown in FIG. 10E and FIG. 10F, which was generated from a scaled-down version of this protocol performed in 12-well plates in which 300 ng of Cre plasmid was transfected into 66000 cells. For experiments depicted in FIG. 10C and FIG. 10D, 1 μg of the respective Cre plasmid was transfected. Briefly, DNA and P3000 reagent (2×DNA amount, 8 μl for 4 μg, etc.) were mixed with 125 μl of OptiMEM. In a separate tube, Lipofectamine reagent (3×DNA amount, 12 μl for 4 μg, etc.) was mixed with 125 μl of OptiMEM. The two tubes were mixed and allowed to sit at room temperature for at least 20 minutes. Transfection mix was added to a gelatinized well of a 6-well plate, and cells were added on top in 2 mL of medium. Medium was changed every day, and treatments such as 0.5 UM Tamoxifen (Sigma) and 20 UM Pifithrin-α (Sigma) were performed at the specified times. For the Pifithrin-α experiment in which Cre-transfected cells were cultured for longer than 72 h, the cell population was split using Accutase (Gibco) and 30% of the cell population was transferred to a new plate at day 3. For the inducible Cre variant experiments, the cell population was split, and 100,000 cells were transferred to a new plate at day 3 and day 5. For various experiments, genomic DNA was prepared from a minimum of 25% of the cell population.

For K562 experiments, 200,000 cells were nucleofected with 1 μg of the respective recombinase plasmid using the Lonza 4D strips in a 20 μl reaction according to the manufacturer's protocol (V4XC-2032). Cells were incubated after pulsing at room temperature for 10 minutes before plating. Cells were split at day 3 post-nucleofection, and 30% of cells were carried forward. The entire cell population was harvested on day 6. Specified cell amounts were plated 3 days post-nucleofection in a fresh 12-well plate to derive K562 bottlenecked populations. Bottlenecked populations were expanded to a 6-well plate when saturated, and samples were collected once saturated in the 6-well plate (>8 days post-nucleofection). For various experiments, genomic DNA was prepared from a minimum of 25% of the cell population.

IVT-seq library construction. An experimental protocol for mapping integration sites using T7 IVT on genomic DNA has been described recently by the group (37). In this example, this protocol is followed with some minor modifications. The same protocol was followed both for shuffle cassette insertion site mapping from the parental population and for rearrangement call validation post Cre transfection. Genomic DNA purified using the Qiagen DNeasy kit was used as a template for the IVT reactions. For IoxPsym+mESC samples, 300 ng of template was used per reaction; for IoxPsym+K562, attB/P+mESC, and attB/P+K562, 500 ng of template was used per reaction. T7 IVT was performed using the HiScribe T7 High Yield RNA Synthesis Kit (NEB E2040S), in a 60 μl total reaction for IoxPsym+mESCs and in a 30 μl total reaction for the other samples. Reactions were incubated at 37° C. for 16 hours in a thermocycler with the lid set to 50° C. Reactions were treated with Turbo DNase (Thermo AM2238) to remove template DNA according to the manufacturer's instructions, and RNA was extracted using TRIzol LS reagent (Thermo 10296010). Briefly, each sample volume was normalized to 250 μl with water, and 750 μl of TRIzol LS reagent was added. Samples were mixed by pipetting and incubated at room temperature for 4 min. 200 μl of chloroform was added, and samples were incubated at room temperature for 3 min. Samples were then spun at 12,000×g for 15 min, and the aqueous phase was transferred to a new tube. 1 μl of 5 mg/mL Glycogen was added (Invitrogen) per sample. Next, RNA was precipitated by adding 1 volume of isopropanol. Samples were mixed by inverting and incubated at −80° C. for 1 hour and subsequently spun at 21,000×g for 1 hour at 4° C. RNA pellet was washed with ice cold 80% ethanol and resuspended in 11.5 μl of H₂O. Reverse transcription was performed using the SuperScript IV Reverse Transcriptase (Thermo 18090200) with 0.5 μL 100 UM RT primer that contains an 8-base-pair degenerate 3′ end (oSP1012). RNA was initially incubated with 0.5 μL 100 UM RT primer (oSP1012) and 1 μL 10 mM dNTP at 65° C. for 5 min and cooled on ice. Enzyme, buffer, RNase inhibitor, and DTT were added, and the reactions were incubated in a thermocycler at 23° C. for 10 min, 50° C. for 15 min, and 80° C. for 10 min, followed by a hold at 10° C.

For IoxPsym+mESC replicate 1, 2nd strand synthesis for both top and bottom strands (FIG. 2B) was performed in the same reaction with primers oSP1008, oSP1021, and oSP1013. For IoxPsym+mESC replicate 2, 2nd strand synthesis for top and bottom strands was performed separately, one with oSP1008 and oSP1013, and another with oSP1021 and oSP1013. Four 50 μl PCR reactions were performed per sample with Q5 Polymerase (NEB M0492S) with the following cycling parameters: 98° C.-3 min; 4 cycles of 98° C.-20s, 65° C.-20s, 72° C.-30s; 72° C.-60s, hold at 4° C. Reactions corresponding to a particular sample and primer pair were pooled. For replicate 1, double-sided size selection (0.5×, 1.1×) was performed with Ampure XP beads (Beckman A63882) on 200 μl of sample and eluted in 50 μl of H₂O. For replicate 2, double-sided size selection (0.5×, 1.1×) was performed on 100 μl of the sample and eluted in 25 μl of HYO. From each sample, a second PCR was set up with indexing primers using 12 μl of the previous eluate as input. Two 50 μl PCR reactions were performed per sample with Q5 Polymerase (NEB M0492S) with real-time tracking using SYBR green dye with the following cycling parameters 98° C.-3 min; 4 cycles of 98° C.-15s, 65° C.-15s, 72° C.-30s until the curves reached saturation (14-16 cycles). Running these libraries on a D1000 ScreenTape (Agilent) revealed a smear of expected size but also some lower molecular weight products that may dominate the sequencing reaction. To address this, 3 equimolar pools were created: samples from replicate 1, top strand samples from replicate 2, and bottom strand samples from replicate 2. Pools were run on a 6% TBE PAGE gel, and DNA between 400 bp and 1000 bp was excised and purified.

For both replicates of IoxPsym+K562, attB/P+mESC, and attB/P+K562 samples, 2nd strand synthesis was performed according to the replicate 2 protocol described above with minor modifications. Two 50 μl PCR 1 reactions were performed per sample, one corresponding to the top strand and one to the bottom strand. After bead clean-up, one 50 μl PCR 2 reaction was performed per sample, per strand. PCR2 was performed with the previous cycling parameters for a total of 16-18 cycles. Libraries were run on agarose gel and DNA between 400 bp and 1000 bp was excised and purified.

Library sequencing was performed on an Illumina NextSeq2000 P2 300 cycle kit. For IoxPsym+mESCs, the read lengths were: 106 read1, 10 index1, 10 index2 and 212 on read2. For the other samples, read lengths were: 124 read1, 6 index1, 10 index2 and 198 on read2.

Amplicon-seq library construction from bulk samples. As detailed in FIGS. 2A-2D, two different strategies for amplicon-seq library construction were employed: 2-primer (data in FIGS. 5A-5G, 3C, 6A-6G, 8, and 9A-9H) and 4-primer (data in FIGS. 10A-10E and the IoxPsym+K562, attB/P+mESC, and attB/P+K562 experiments). The major difference between these two strategies is that either 2 or 4 primers corresponding to the capture sequences were used to generate the PCR product. In the case of the 2-primer strategy, the P5 Illumina sequencing adapter can only come from the primer that binds CS2, and the P7 adapter can only come from the primer that binds CS1. This precludes identification of recombined shuffle cassettes that contain the same capture sequence on both sides of the IoxPsym site (FIGS. 2C-2D). In the 4-primer strategy, primers with P5 and P7 adapters that bind to both CS2 and CS1 are included in the PCR. Theoretically, this would enable the detection of recombined shuffle cassettes with the same capture-sequence on both sides. However, these events were not detected even in the case of the 4-primer strategy, probably due to suppressive PCR (FIGS. 2C-2D, 10A-10B).

For the 2-primer experiments, 250 ng of genomic DNA were used, prepared using the Qiagen DNeasy kit as input. PCR1 (UMI addition) was performed in a 50 μl reaction with Q5 Polymerase (NEB M0492S), with 2.5 μL of each 10 μM primer (oSP1008 and oSP997), with the following cycling parameters: 98° C.-5 min; 4 cycles of 98° C.-20s, 62° C.-20s, 72° C.-30s; 72° C.-60s. PCRs were cleaned up using AmpureXP beads (1×) and eluted in 11 μl of H₂O. PCR2 (sample indexing and sequencing adapter addition) was again performed with Q5 Polymerase in a 50 μl reaction using 10 μl of eluate from the previous step as template and 2.5 μL of each 10 UM sample indexing primer. Progression of PCR2 was monitored in real-time using SYBR green dye and reactions were stopped before saturation (usually 15-18 cycles). Cycling conditions for PCR2: 98° C.-5 min; 15 cycles of 98° C.-10s, 65° C.-10s, 72° C.-20s. Reactions were cleaned up with AmpureXP beads (1×) and eluted in 12 μl of H₂O. Sample quality was confirmed on a TapeStation D1000 ScreenTape (Agilent), after which reactions were pooled. Sequencing was performed on an Illumina NextSeq2000 100-cycle kit with the following read lengths: 69 read1, 6 index1, 10 index2, and 53 on read2.

For the 4-primer experiments shown in FIGS. 10A-10E, 100 ng of genomic DNA prepared using the Qiagen DNeasy kit was used as input. For the 4-primer experiments involving IoxPsym+K562, attB/P+mESC, and attB/P+K562, 250 ng of genomic DNA prepared using the Qiagen DNeasy kit was used as input. PCR1 was performed with a mix of 4 primers (oSP1008, oSP997, oSP1021, and oSP1022) with 0.125 μl of each primer at 100 UM per 50 μl reaction. The rest of the protocol was identical to the 2-primer workflow described above.

Single-cell sorting and construction of amplicon-seq libraries. Cre reporter (pSP0767 pLV-FIox-BFP-dsRed) was cloned using pLV-fIox-dsRed-GFP as a template using Gibson assembly (65). 200,000 cells were reverse-transfected with 1 μg of Cre or Bxb1 recombinase and 200 ng of the reporter in a 6-well plate per reaction as described above. Two transfections were performed per recombinase. One set of cells per recombinase was treated with 20 μM Pifithrin-α 24 hours post-transfection for a total time of 48 hours. Cells were harvested at 72 hours post-transfection and FACS sorted on the activity of the Cre reporter at single-cell purity into gelatinized 96-well plates containing growth medium with or without Pifithrin-α. FACS data were analyzed using FlowJo.

Clones were allowed to grow out for 9 days before cells were frozen for genomic DNA extraction in 96-well plates. Genomic DNA was extracted in the 96-well format using the Quick-DNA/RNA MagBead kit (Zymo Research R2130) according to the manufacturer's instructions. Amplicon-seq libraries were constructed from 9.8 μl of template genomic DNA (estimated to be between 10-50 ng) per well using the 4-primer strategy described above. PCR1 was performed in a 20 μl Q5 Polymerase reaction with 0.05 μl of each primer at 100 μM. After 1× clean up with Ampure XP beads and elution in 11 μl of H₂O, PCR2 was performed on 10 μl of eluate in a 25 μl Q5 Polymerase reaction with 1.25 μl of each indexing primer at 10 μM. After 18 cycles, 10 μl of each reaction was pooled and purified using a Zymo Research Clean and Concentrate kit. The sample was eluted in 100 μl of H₂O, run on an agarose gel, and the band of the appropriate size was excised and purified (Zymo Research D4007). Libraries were run on an Illumina NextSeq 2000 200-cycle kit.

Preparation of cells and libraries for single-cell RNA sequencing. For the IoxPsym+mESC scRNA-seq experiment described in FIGS. 14A-14C, 300,000 parental cells were transfected in 6-well plates as described above with 1 μg of Cre and 200 ng of the reporter per transfection. 10 individual wells were transfected. At 24 hours post-transfection, 5 wells were treated with 20 UM Pifithrin-α for 48 hours total. At 72 hours post-transfection, both Pifithrin-α-treated and untreated cells were harvested, and 500,000 RFP positive cells were FACS sorted into a combined tube based on the activity of the Cre reporter. Untransfected parental cells were harvested, and 1 million cells were used as input in parallel with Cre-sorted cells for the protocol.

For the attB/P+K562 and attB/P+mESC “Lane 1” experiment described in FIGS. 15A-15H, 200,000 parental cells were nucleofected (2 reactions) or transfected (3 reactions) with 1 μg of Bxb1 plasmid as described above. Cells were harvested at day 3 post-transfection and used as input for the protocol. For the “Lane 2” experiment, indicated bottlenecked populations were thawed and split once before being used as input in the protocol. For both Lanes, 500,000 cells from each population were mixed as indicated in FIG. 15A and were used for the subsequent steps.

After washing twice with cold 1×PBS (Gibco), 1 million cells per sample were resuspended in 400 μl of cold PBS. Cells were fixed with 1600 μl of cold 100% methanol, added dropwise with swirling. Cells were left on ice to fix with gentle swirling to mix every 5 minutes during the incubation. Cells were rehydrated with 4 mL of cold 1×PBS, added slowly with gentle swirling of the tube. Cells were spun down and resuspended in 60 μl of PBS. For the IoxPsym+mESC experiment, 60,000 cells and 380,000 cells in total were counted in the Cre and parental samples, respectively, using a Countess automated cell counter (Thermo), of which all and 100,000 cells in 18 μl of PBS were used for T7 IVT, respectively. For the attB/P+K562/mESC experiment, 600,000 and 450,000 cells were counted, of which 100,000 cells per sample in 18 μl of PBS were used for T7 IVT. IVT reactions were set up in 30 μl total volume with the HiScribe T7 High Yield RNA Synthesis Kit (NEB E2040S) with 2 μl of each NTP, buffer, and enzyme. Reactions were incubated at 37° C. for 1 hour in a thermocycler. Cells were immediately placed on ice, and 20 μl of cold PBS was added to each sample. 40,000 cells were processed per lane of a 10× Genomics Single Cell 3′ HT with Feature Barcoding kit.

Transcriptome libraries were prepared as per the manufacturer's protocol. To prepare libraries from T7 transcripts captured using CS1 and CS2, the supernatant from the clean-up after cDNA amplification of the standard 10× Genomics feature barcoding protocol was started with for the IoxPsym+mESC experiment. For the attB/P+K562/mESC experiment, two construct-specific primers (oSP1059, oSP1060) were spiked in at 0.5 UM into the cDNA amplification step. Two rounds of PCR were performed with primers specific to the shuffle cassette. In PCR1, oSP997 (CS1-TruSeq2), oSP1022 (CS2-TruSeq2), and oSP1061 (feature-cDNA primer F) primers were used (1.25 μl of 10 μM each) in a 50 μl Q5 polymerase reaction with 5 μl of template. Cycling conditions: 98° C.-45 s; cycles of 98° C.-20s, 60° C.-5s, 72° C.-5s; 72° C.-60s, 4° C.-hold. 15 cycles of PCR1 were performed for the IoxPsym+mESC experiment and 12 cycles for the attB/P+K562/mESC experiment. IoxPsym+mESC were cleaned up with AmpureXP beads (1×) and eluted in 15 μl of Qiagen buffer EB. attB/P+K562/mESC reactions were cleaned up with AmpureXP beads (1×) and eluted in 30 μl of buffer EB. In PCR2, 5 μl of PCR1 eluate was used as input into either one or two (IoxPsym+mESC experiment) 50 μl Q5 Polymerase reactions per sample with sample index primers (1.25 μl of 10 μM each) that add Illumina adapters. Reaction progress was monitored using SYBR green and was stopped before saturation. Cycling conditions for the IoxPsym+mESC experiment: 98° C.-5 min; 9 cycles of 98° C.-10s, 65° C.-10s, 72° C.-20s; 72° C.-60s, 4° C.-hold. Cycling conditions for the attB/P+K562/mESC experiment: 98° C.-1 min; 8 cycles of 98° C.-20s, 63° C.-20s, 72° C.-1 min; 72° C.-60s, 4° C.-hold. After a clean up with AmpureXP beads (1×), the quality of the libraries was confirmed (prominent single-peak) by running them on a TapeStation D1000 ScreenTape (Agilent).

For the IoxPsym+mESC experiment, T7 libraries were sequenced on an Illumina NextSeq2000 100-cycle kit with the following read lengths: 28 read1, 6 index1, 8 index2, and 96 on read2. Transcriptome libraries were sequenced on two separate NextSeq2000 100 cycle runs, initially with 28 read1, 10 index1, 10 index2 and 90 on read2 and next with 28 read1, 6 index1, 8 index2 and 96 on read2.

For the attB/P+K562/mESC experiment, T7 libraries were sequenced on an Illumina NextSeq2000 100-cycle kit with the following read lengths: 75 read1, 6 index1, 10 index2, and 47 on read2. Transcriptome libraries were sequenced on a NextSeq2000 P3 100-cycle kit with the following read lengths: 28 read1, 10 index1, 10 index2, and 90 on read2.

IVT-seq data analysis for insertion-site mapping in the parental population. The analysis pipeline was based on the pipeline published in Li et al., Chromatin context-dependent regulation and epigenetic manipulation of prime editing. Cell187, 2411-2427.e25 (2024). Briefly, reads were demultiplexed using bcl2fastq (v2.20.0.422). Read1 contains the identity of the shuffle barcodes, whereas read2 contains the associated genomic sequence. Reads were passed through a custom script (IVTextractBCs.py) to extract barcode sequences based on exact matches to the expected preceding and subsequent bases. The strand of each read (top-CS2 or bottom-CS1) was also assigned at this step. PiggyBac ITR sequences were trimmed from read2 using cutadapt (v2.5) with the following parameters:—cores=4—discard-untrimmed-e 0.2-m 10-a CCCTAGAAAGATA (SEQ ID NO: 4) (66). Trimmed reads were mapped to either the mm10 or hg38 reference genome using bwa mem (v0.7.17) with the −Y option (67). SAM files were sorted using samtools (v1.9) and filtered out reads that do not align to known PiggyBac insertion sites (TTAA) or align to several locations (contain XA:Z flag) using a custom script (align_filter.py) (68). Filtered SAM files were converted to BED format using the sam2bed tool in bedops (v2.4.35) (69).

For mESC samples, BEDtools (v2.29.2) intersect was used with the -loj-wa-wb-filenames-sorted options to extract those alignments overlapping with a known variant between the BL6 and CAST alleles from the Sanger Mouse Genome database. Keane et al., Mouse genomic variation and its effect on phenotypes and gene regulation. Nature 477, 289-294 (2011). Quinlan & Hall, BEDTools: a flexible suite of utilities for comparing genomic features. Bioinformatics 26, 841-842 (2010). A custom python script (cleanup_sort_variantcall_update.py) was used to parse the CIGAR string of each alignment intersected BED file to assign each read to each of the following categories: BL6 (read overlaps with variant and sequence of alignment at that position matches the reference allele), CAST (read overlaps with variant and sequence of alignment at that position matches variant allele) and ‘no-Variant’ (read does not overlap with variant). Inconclusive alignments that contained some incongruence in allele assignment were discarded. The position of each alignment was determined by the genomic strand it mapped to. If the strand is +, the position is at the end of the alignment; conversely, if the strand is −, the position is at the start of the alignment. For K562 samples, alignments were sorted and assigned to a position using a custom Python script (cleanup_sort_variantcall_update_K562.py).

Next, another custom Python script (ivt_clustered_groupcollapse_iterable.py) was used to collapse alignments into groups based on: 1) a shared chromosome and position, and 2) a shared set of barcodes extracted from read1. Alignments at a given position were clustered together if their associated barcodes were within a Levenshtein distance of 6. Within each cluster, the most common values for barcode1, barcode2, strand of alignment to genome (+ or −), and shuffle cassette strand (top-CS2 or bottom-CS1) were assigned as the representative values for that cluster. For mESCs, each cluster was assigned to an allele based on the most common allele value within, without taking into account the number of no-Variant assignments within that cluster. Total read count (number of alignments per cluster), total read1 UMIs (coming from forward primer of second strand synthesis), total read2 UMIs (first 8 bp of genome coming from the degenerate 3′ end of the RT primer), and total number of unique alignment lengths per cluster were also determined.

To define the list of parental insertions, the outputs from the previous step from replicate 1 and replicate 2 were merged based on shared position, barcode, and strand values. Within this merged dataset for mESCs, for each cluster of alignments, allele value was assigned based on the allele value in replicate 1 and replicate 2, without considering no-Variant assignments. In the case that replicate 1 and replicate 2 allele assignments were incongruent, the allele for that cluster was assigned as ‘inconclusive’. Clusters with <6 reads and <3 unique lengths aligned in replicate 2 of the parental sample were filtered out. Clusters containing barcode combinations that did not map uniquely to a genomic location were then filtered out. Within this set, bona fide insertions were defined by: 1) a pair of read clusters whose alignment position differs by exactly 4 bp, 2) the first cluster in the pair maps to the −strand and the second cluster maps to the +strand, 3) the pair does not encode the same shuffle cassette strand (top-CS2 or bottom-CS1); and 4) the barcodes detected in each member of the pair are reverse complement of one another.

Within this set for mESCs, the allele value for each insertion was assigned as inconclusive in the case that the allele assignment for the pair of read clusters that make up the insertion was incongruent. In the case that one of the clusters was marked as no-Variant, the allele of the other cluster in the pair was considered the allele of that insertion. Insertions were merged with amplicon-seq data from the parental cells based on shared barcode pairs. Only those insertions whose barcodes could be detected in the amplicon-seq data from the parental cells were kept. In some cases, there were a small number of remaining sites that had more than one pair of barcodes called at that site. By manual observation, these seemed to arise from a clustering artifact and ought to have been collapsed into one cluster. The row was chosen that had the higher value in the number of unique lengths aligned in rep2 on the left side of the insertion at these positions for the final set.

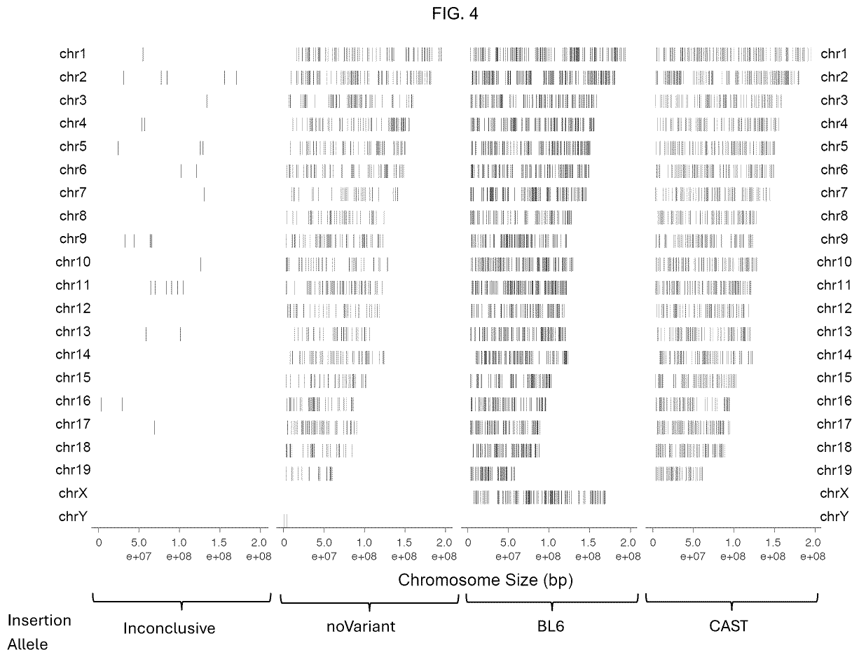

To generate the visualization in FIG. 3A, alignments were visualized in IGV 2.16.1 (71) and BED files containing insertions assigned to each allele were loaded separately as tracks. The visualizations shown in FIG. 3D and FIG. 4 were generated using the ChIPseeker package in R (72). Other plots were made using a combination of matplotlib (3.8.1) and seaborn (0.13.0) libraries in Python.

Amplicon-seq analysis and rearrangement calling. Reads were demultiplexed using bcl2fastq (v2.20.0.422). Reads were passed through a custom script (read_extract_iterable.py or read_extract_iterable_4primers.py or amplicon_BCExtract_20240906.py) to extract barcode sequences and UMIs based on matches to the expected preceding and subsequent bases. In the case of 4-primer amplicon-seq, the strand of each read (top-CS2 or bottom-CS1) was also assigned at this step. The identity of the recombination site (IoxP, attB, attP, attL or attR) was determined from the combination of bases specific to each site expected to be detected in read 1 and read 2. The total number of reads per barcode pair was taken as the readcount and the total number of unique UMIs detected was taken as the UMI count (df_group.py or df_group_4primers.py). In 4-primer amplicon-seq, the same shuffle cassette can result in two distinct amplicons. Reads coming from the same shuffle cassette based on the shared set of barcodes were collapsed, and the read counts and UMI counts were summed up. The barcode pairs of both types of amplicons were not detected and were discarded. In various cases, the barcode closest to CS2 was named barcode1, and the barcode closest to CS1 in the shuffle cassette was named barcode2. Reads and UMI counts were normalized for sequencing depth. For the IoxPsym+mESC parental barcode set, normalized read and UMI counts were averaged across 4 replicates and used for defining the bona fide set of insertions as detailed in the section above. For the remaining parental samples, normalized read and UMI counts were averaged across 2 replicates and used for defining the bona fide set of insertions as detailed in the section above.

Amplicons with shared barcode pairs that contained the same capture sequence were searched for. To eliminate confounding through errors in PCR or sequencing, the search was restricted to those amplicons containing barcodes that were both found in the bona fide list of parental insertions. The amplicons that contained the same barcode (that is, within Levenshtein distance of 6) on both sides of the IoxPsym site were also eliminated. CS1-CS1 and CS2-CS2 amplicons were not readily detected in the data, presumably due to suppressive PCR (FIGS. 2A-2D).

For the amplicon-seq data generated from single-cell sorted clones, wells with fewer than 100 k reads were discarded. Barcode sequences were extracted, and read/UMI counts per barcode pair were determined as above. The set of barcodes associated with each well was determined as the barcode pairs whose readcounts were >1 standard deviation above the mean readcount for barcode pairs detected in that well using the zscore function in the scipy.stats library. Analysis was then restricted to those barcode pairs that were present in the bona fide parental insertion list (n=5088). Barcode pair count per well was normalized for the number of clones observed in that well by eye.

Rearranged barcodes were identified by comparing the set of identified barcode pairs in the dataset to the bona fide list of parental insertions. Both barcodes in the rearranged pair were in the bona fide list but were not found together in the parental cells. To remove any artifacts caused by PCR chimeras or errors, each rearranged barcode pair was present at ≥2 UMI. The nature of each rearrangement denoted by a rearranged barcode pair was inferred based on the position and orientation of the parental insertion sites (FIGS. 1A-1E and 2A-2D). It was determined that inter-homolog translocations were rare (recombination between shuffle cassettes on the same chromosome but assigned to different alleles). Therefore, deletions were parsimoniously identified as those rearranged barcode pairs between shuffle cassettes on the same chromosome that were inserted in the same orientation. In a similar manner, inversions were identified as those rearranged barcode pairs between shuffle cassettes on the same chromosome that were inserted in the opposite orientation. The size of the rearrangement was calculated as the difference between the positions of the two original insertion sites. Translocations were those rearranged barcode pairs found between shuffle cassettes on different chromosomes.

Deletions could be further classified as coming from the genomic copy or the extrachromosomal circle based on the barcodes that were detected (FIGS. 1A-1E and 3A-3D). For example, considering the case of a deletion between two shuffle-insertions X and Y at pos N and pos N+100 on a chromosome, with ‘top-CS2’ orientation (CS2 found closest to the left of the chromosome). The ecDNA would contain the CS2 barcode of insertion Y and the CS1 barcode of insertion X, and the genomic copy would contain the CS2 barcode of insertion X and the CS1 barcode of insertion Y. In the case of deletions between two ‘bottom-CS1’ insertions with (CS1 found closest to the left of the chromosome), the opposite would be true.

Translocations could also be further classified as balanced and unbalanced. Barcode pairs corresponding to unbalanced translocations could further be assigned as leading to either an acentric or dicentric chromosome (FIG. 10G). The relative position of each translocation breakpoint to the centromere was determined. Based on the orientation of the insertion (top or bottom strand), each barcode was assigned to be centromere proximal or distal. If both barcodes corresponding to the detected translocation were centromere proximal, the barcode combination was classified as having originated from a dicentric chromosome. If both barcodes were centromere distal, the translocation was classified as an acentric. In other cases, the translocation was a balanced translocation.

All Circos plots were made using the pyCircos library (73). The remaining plots were made using a combination of matplotlib (3.8.1) and seaborn (0.13.0) libraries in Python.

Rearrangement call validation using IVT-seq data. IVT-seq libraries were constructed using the same genomic DNA samples used to prepare amplicon-seq libraries from Cre-transfected samples. The data was pushed through the same analysis pipeline as described above for IVT data from parental cells, until the collapsing of alignments into groups based on their shared barcodes and positions. For each rearrangement detected in the amplicon-seq data, it was determined whether there was at least one transcript detected in the IVT-seq data from that sample that supported the rearrangement call (FIG. 6D). The fraction of rearrangements supported in each IVT replicate from each sample was plotted using the matplotlib (3.8.1) library in Python.

Single-cell data analysis. Initial pre-processing and quality filtering. 10× Genomics 3′ gene expression (transcriptome) libraries were processed using cellranger-6.0.1 count function (with reference refdata-cellranger-mm10-3.0.0 for mESC cells [IoxPsym+mESC experiment 1 and attB/P+mESC/K562 experiment lane 1] and refdata-gex-GRCh38-2020-A for K562 cells [attB/P+mESC/K562 experiment lane 1 & 2]). Resulting raw count matrices were converted to a Seurat (v4.3.1) (74) object using functions Read10× and CreateSeuratObject (options: min.cells=3, min.features=50). The mitochondrial fraction was computed, and cell barcodes with >1500 and >1000 transcriptome UMI/cell (K562 and mESC, respectively) and with 2-8% and 1.3-6% mitochondrial fraction (K562 and mESC, respectively) (FIG. 16A) were retained. For IoxPsym+mESC experiment 1, cell barcodes with >1000 transcriptome UMI/cell and with 1-12% mitochondrial fraction (FIGS. 14A-14C) were retained.

Scrublet 0.2.3 (75) was run on filtered cells, and cells with a doublet score <0.4, leaving 12460 and 8499 (K562 and mESC, respectively, lane 1) and 20800 (K562, lane 2) high-quality cells for downstream analysis.

For the lane 1 mESC+K562 barnyard experiment, the following criteria were further used to unambiguously assign species identity to cells: cell barcodes passing each separate species singlet processing thresholds were retained, and any cell barcodes removed as a putative scrublet-called doublet in one species but not the other were further marked as doublets (e.g., cell called as singlet in K562 mapping but flagged as a doublet in mESC mapping). Total transcriptome UMIs mapping to both species from these cell barcodes were then inspected, showing clear separation between singlet and doublet. Initial assignment from the single species threshold was finely refined to stringently identify cells as doublets if transcriptome UMI ratios were not sufficiently separate (threshold selected by inspection): mm10/hg38<1.42 or hg38/mm10<3, leaving a final set of 11117 K562 and 7307 mESC cells (1172 assigned putative species doublets).

The samples displayed a cleanly separated cell population in the total transcriptome vs. mitochondrial fraction plane, including cells with high confidence rearrangements (FIG. 16A), indicating good cellularity despite fixation and IVT treatment to generate the T7-BC prior to emulsion and library generation.

Generating T7-shuffle BC count matrix. To obtain the BC-by-cell-count matrix, the fastq files were first modified to separate the cell barcode and shuffle barcodes in two distinct files (original read structure—read 1: first 28 cycles [cell barcode]-umi, remaining cycles 29 to 75 to read [capture sequence]-BC-[att site]; read 2: cycles 1 to 47 cycles to [read capture sequence]-BC-[att site]). Modified fastq content—read 1:28 cyclces [cell barcode]-umi, read 2: cycles 1 to 47 cycles to [read capture sequence]-BC-[att site] followed by original cycles 29 to 75 from original read 1. The merge was generated using seqtk version 1.4 and the following script (appending line number to join and retaining only desired information in final fastq (seqtk trimfq-b 28 file_R1_001.fastq.gz|join<(zcat file_R2_001.fastq.gz|nl)<(cat-|nl)|awk-F′ ′ ′{if ($2~/\hat{ }@VH00979/) {print $4″ ″$5;}else {print $2$3;}|gzip>file_w_47bpR1R2_transfer_R2_001.fastq.gz).

T7-BC sequencing data were then processed using cellranger-6.0.1 count to perform error correction on the cell barcodes. The unmapped reads with error-corrected cell barcodes were selected from the sorted BAM file output, and barcode sequences were extracted for each read from the shuffle cassette by looking for matches for constant surrounding sequences. From the read 1 portion (searching in cycles 48 to 95 appended to read 2 as described above): attP CS2 if TGAGC (. {20}) GTGG, attB CS2 if TGAGC (. {20}) GGCC, attP CS1 if AAAGC (. {20}) TGGG, attB CS1 if AAAGC (. {20}) CCGG. From the original read 2 (searching in cycles 15 to 47): attB CS1 if AAAGC (. {20}) CCGG, attP CS1 if AAAGC (. {20}) TGGG, attP CS2 if AAAGC (. {20}) TGGG, attB CS2 if TGAGC (. {20}) GGCC. The resulting two (barcode)-(capture sequence)-(recombinase site) combinations were stored and joined to the error-corrected cell barcode and umi from the read. Reads counts and total set of UMIs for the cellBC/BC1/BC2/capture/recombinase site combinations were then tallied, discarding likely chimeric UMIs (taken to be UMIs for which the proportion of reads associated with a given BC1/BC2/capture-orientation of the other BC1/BC2/capture/recombinase site in the specified cell barcode, falls below 0.2). The number of error-corrected UMIs for a given BC1/BC2/capture/recombinase site was then taken as the number of connected components in a graph created by connecting the UMIs associated with that combination with a Hamming distance≤1. To filter out spurious molecules, sequencing errors or PCR chimeras, only cell barcode BC1/BC2 combinations with reads/UMI ≥15 and >9 for Lane 1 and Lane 2, respectively, were retained commensurate with the level of sequencing saturation in both libraries.

De novo identification of clonotypes. High MOI (many T7-BC pairs per cell) and clonal nature of the population to identify clonotypes (defined as the full set of BC pairs within a clone) were leveraged directly from the single-cell data T7-BC data, working under the assumption that co-detection of T7-BC pairs should only happen from barcodes within the same clone, given the high complexity of starting libraries. Lalanne et al., Multiplex profiling of developmental cis-regulatory elements with quantitative single-cell expression reporters. Nat. Methods 21, 983-993 (2024). Computational identification of clonal cells in single-cell CRISPR screens. BMC Genomics23, 135 (2022). Ribeiro-Dos-Santos et al., Genomic context sensitivity of insulator function. Genome Res.32, 425-436 (2022).

UMI counts were summed in the same cell with the same BC1/BC2 pair but captured from different capture sequences. To avoid improper tally of re-arranged barcodes, only the BC associated with CS1 was used for clonotype identification. The T7-BC UMI counts were then subsetted to those originating from the set of quality-filtered cells as described above, and retained barcode pairs with ≥3 UMI per barcode per cell. To further remove cells and barcode pairs with little signal for the purpose of de novo clonotype identification, any cells with <11 total T7-BC UMIs (after the >3 UMI/BC per cell thresholding) and T7-BC pairs with <11 total UMIs across the cells were removed. A count matrix was then constructed and Seurat v4.3.1 was used to perform dimensional reduction (NormalizeData with normalization.method=“RC”, scale.factor=10000, with FindVariableFeatures selection method=“vst”, nfeatures=length (T7_BC), RunPCA on the top 100 PCs with identified variable features, FindNeighbors with k.param=10, and FindClusters at resolution=1). The resulting communities in T7-BC space were used to identify the putative set of shuffleBC cassettes per clonotype.

To do so, T7-BC representation across the cells assigned from clustering in the BC space was calculated as the proportion of cells in the cluster with the barcode pair detected. These proportions were then rank ordered, and a heuristic threshold was used to demarcate barcode pairs associated with a clone: the threshold was set as the fold-change in representation from rank n BC pair to rank n+1 barcode pair became higher than 2 (Lane 1) and 1.5 (Lane 2) or 0.075 (Lane 1) and 0.1 (Lane2) whichever was highest, empirically selected as a reliable marker of the inflection point in the distribution. The resulting set of putative clonotypes was further filtered by retaining only clonotypes for which the maximum detection fraction (from the top T7-BC pair for that clonotype) was above 0.45 (Lane 1) and 0.55 (Lane 2), and in which >1 T7-BC pair was detected. In addition, the subsetted T7-BC count matrix for each putative clonotype (with only assigned cells and contained T7-BCs) was inspected, and any clonotype corresponding to a clear doublet (split in the matrix into two blocks) was not retained for downstream analysis.

As a final quality control step, because this procedure tends to redundantly create multiple clusters for the same clonotype (depending on the resolution parameter), the Jaccard index (on the set of T7-BCs) was computed for each identified clonotype pair. For Lane 1, the distribution of Jaccard indices was strongly bimodal, with the majority of pairs displaying 0 overlap, with a small minority with a Jaccard index of >0.5, suggesting identical underlying clonotypes. A graph was created with nodes corresponding to putative clonotypes and connected if their Jaccard index was >0.5. The union of the T7-BC from the connected clusters (mostly singletons) was then taken as the clonotypes. All in all, this led to 91 high-confidence de novo clonotypes from round 1 (see below for round 2), with a mean MOI of 31.4. For Lane 2, a similar analysis of Jaccard similarity revealed a set of clonotype-assigned barcodes that were broadly shared across nearly all clonotypes. Given the clonal representation in the population (two clones with >2000 cells each, making up nearly 45% of the cells in the dataset), it was suspected that the shared barcodes originated from these highly represented clones. Calculating the mean UMI per BC per putative cluster in the T7-BC space indeed confirmed that the highly represented clusters were detected at a much higher proportion (mean 4 UMI/cell compared to 1.2 UMI/cell for other clusters) in one of the large clusters. To avoid these barcodes from large clones being spuriously included in the clonotype calls, Lane 2 analysis was repeated after excluding cells and barcodes assigned to these two large clones in the first pass. Procedure for the large-clone excluded set for Lane 2, then proceeded as before, leading to 53 high-confidence clones (including the two large clones), with a mean MOI of 26.5.

An iterative round of assignment and mapping to identify additional clonotypes. In order to comprehensively identify clonotypes, an iterative approach was performed whereby cells were first assigned to clonotypes (see below), as described using the round 1 clonotypes described above. Any assignable cell was then removed from the de novo pipeline described previously, and the process was repeated. Doing so generated an additional set of 50 (final 141 total, Lane 1) and one (final 53 total, Lane 2) high-confidence clonotypes.

Single-cell assignment of cells to clonotypes. To obtain a more sensitive assignment of cells to clonotypes (in contrast to the clustering in a dimensionally reduced barcode space used for de novo clonotype calling as described above), the T7-BC detected (considering only events with ≥2 UMI per barcodes pairs per cell, after summing pairs captured from both CS1 and CS2, see barnyard and clonotype precision/recall analysis below) were compared in the cells to the set of high-confidence clonotypes. In order not to bias against re-arranged cells for the purpose of assignment to clones, only the CS1 barcode was considered for this analysis. For each cell-clonotype pair, the fraction of cell-detected barcodes belonging to the top clonotype (precision) was recorded, in addition to the fraction of barcodes from the clonotype recovered in the cell (recall). Across various cells, the clonotype with the highest precision (similar result if selecting on recall) was retained as the selected candidate (top_precision, top_recall). The resulting distribution of top-scoring clonotype precision/recall assignments displayed enrichment in high precision values with a range of recall. Cells in that plane were considered assignable to a clone with high confidence if top_recall>0.1 and top_precision>0.75. To further remove the possibility of doublets, any resulting assigned cells with second-top assigned clones showing recall >0.1 were removed from the set of high-confidence assignments. For Lane 2, as a result of the highly represented clonotypes ‘emitting’ shuffleBC IVT transcripts at a substantial level in the ambient mixture that were captured in other droplets (see discussion in clonotype reconstruction section), a number of cell assignments to less well represented clonotypes were initially called as ‘low purity’. To circumvent this issue, the assignment to clonotypes was repeated, but barcodes originating from these large clonotypes were excluded. Any cell that was initially assigned as low purity (assignment with large clonotype barcodes) and subsequently as high purity (assignment without large clonotype barcodes) was putatively retained as a high confidence assignment. 2797/19987 cells with at least one BC with >2 UMI detected fell in that category, underscoring an opportunity for optimization in future iterations to wash cells and remove non-cell associated IVT products prior to encapsulation.

In the end, for Lane 1, 65.2% (11252/17266) of cells with at least one BC pair (with ≥2 UMI) of cells were assignable to a clonotype. Of the remaining cells, 3526 displayed low capture (recall <0.1) either as a result of missed clonotypes from the reconstruction procedure or from low levels of IVT for the T7-BC generation. The remaining cells, where the set of detected T7-BCs are not predominantly from a single clonotype, either originated from droplets encapsulating doublets or large quantities of ambient RNA. From the original set of 143 clonotypes, 128 had more than 10 cells assigned to them and were considered for the precision/recall analysis. Further, 5 clonotypes had 0 highly confidently assigned cells, suggesting mixed or otherwise low-quality clonotypes in the de novo reconstruction procedure. Of note, these were “round 2” clonotypes that tend to have low MOI, rendering assignment more challenging.

For Lane 2, 73.7% (14727/19987) of cells with at least one BC pair (with ≥2 UMI) of cells were assignable to a clonotype. Of the remaining cells, 2156 displayed low capture (recall <0.1), and the rest low purity. 52/53 clonotypes had >10 cells assigned at high confidence.

Precision-recall analysis for T7-shuffle BC detection. In order to assess which thresholds to use to identify high-confidence detections of rearrangement events, cross-detection analysis was performed in the barnyard experiment (Lane 1, FIG. 15A).

First, at a coarse level, the fraction of shuffleBC known to be present in the mESC population (bulk amplicon sequencing, n=3058 BCs) that were captured was assessed in K562 cells in the scRNA-seq data (and vice-versa, n=11166 BCs by bulk amplicon sequencing of the K562 population). Specifically, only shuffle BC in the bulk amplicon sequencing set was used for the analysis (corresponding to 97.8% of scRNA-seq detected UMIs). Then, cells that were unambiguously assignable to a single species (see processing of scRNA-seq above) were retained. For varying UMI threshold values (1 to 10), the number of UMIs from the K562-BCs and mESC-BCs sets detected in these species-singleton cells (either K562 and mESC) was computed. Barnyard plots (FIGS. 15B and 17A) highlighted a near complete absence of cross-detection for detection events at ≥2 UMI (>98% and >99% UMIs detected in the cognate species at a threshold of ≥2 and ≥3, respectively, FIG. 17B).

Since the cross-species analysis above still calls a spurious detection valid in nearly half of the cases (e.g., shuffleBC emitted in the ambient medium from an mESC clonotype and detected in an mESC cell from another clonotype), a more stringent performance assessment was sought. To do so, high-confidence assigned cells to major clonotypes (>10 cells assigned) were considered, and precision and recall values for varying UMI thresholds were determined. At each UMI threshold, the precision and recall values were averaged over the high-confidence cell-to-clonotype assignments. The curves displayed a sharp increase at 2 UMI (1 UMI mean precision 0.40 to 2 UMI mean precision of 0.92, FIG. 17C) with a modest decrease in recall (0.55 to 0.33), providing solid empirical ground for taking the threshold of 2 UMI as associated with a <8% FDR. The cross-detection performance is a function of the statistical distribution of shuffleBC ‘emitter’ source, as exemplified with the added background resulting from the highly-represented clonotypes in the Lane 2 data.

Single-cell expression analysis. The cell×gene count matrix was normalized by the library size of each cell using Scanpy (Wolf et al., SCANPY: large-scale single-cell gene expression data analysis. Genome Biol. 19, 15 (2018)) after removing genes that were expressed in <10 cells. Fold-changes in normalized expression were compared between the rearranged and non-rearranged groups of cells within the same clone, and these fold-changes were plotted against genomic coordinates using gene annotations from Gencode v38 (Frankish et al., GENCODE 2021. Nucleic Acids Res. 49, D916-D923 (2021)) (FIG. 15F). The statistical significance of the reduction in expression due to the deletion was assessed using a one-sided Wilcoxon ranksum test. This test was also performed for each of the three genes in the deleted region for various sample sizes of rearranged cells. Cells were randomly sampled without replacement from the full set of cells with the confidently detected deletion, and this was repeated for 100 trials per sample size to get an estimate of the variability in statistical significance as it relates to sample size. The fold-changes for the same analysis were plotted and calculated as before (FIG. 21C).

Rearrangement, calling, and visualization from single-cell data. From the set of T7-BCs identified in the single-cell data, rearrangements were called as described above for bulk amplicon-seq. The rearranged BC pairs identified in the dataset were filtered to those present at >2 UMI, in the cells of the correct species in Lane 1, to those in cells assigned to a clonotype, and finally to those rearrangements consistent with the clone of the cell they were detected in (FIGS. 19A-19B). The subsequent analysis focused on the validated set of rearrangements. Circos plots were made using the pyCircos library. Other plots were made using a combination of the matplotlib (3.8.1) and seaborn (0.13.0) libraries in Python.

### Experimental Example 2. Split-Marker Selection and Optimized Mapping System

To enrich cells containing SVs, a split selectable marker system is designed, in which a fully functional marker, such as a resistance gene or a fluorescent protein, is reconstituted only upon recombination. To ensure that only cells undergoing successful recombination are analyzed, a functional marker, such as a drug resistance gene or a fluorescent protein, is reconstituted from two or more non-functional “half-marker” cassettes.

As shown in FIG. 27, these cassettes are designed to be compatible with high-throughput mapping by embedding specified regulatory and tracking elements within a synthetic intron. Specifically, phage polymerase promoters, recombinase sites (e.g., Bxb1, Cre, or Flp), and internal barcodes can be positioned within a synthetic intron. This configuration ensures that these cassette features remain functionally inert during initial transcription, thereby leaving the coding sequence of the selectable marker (e.g., BsdR or GFP) intact and translatable only upon successful genomic rearrangement.

The system employs an optimized promoter architecture designed to maximize transcription efficiency and minimize biochemical interference. The cassettes optimize the sequence context around the T7 promoter and replace one T7 promoter with a T3 promoter. An additional SP6 promoter is added for initial mapping. This approach addresses several technical bottlenecks:

Sequence Context Optimization: The flanking sequences of the phage promoters (e.g., the T7 promoter) are optimized to exclude inhibitory motifs, thereby ensuring high-level transcript yield for downstream sequencing.

Transcriptional Interference Mitigation: By utilizing different phage polymerases (e.g., replacing a convergent T7 promoter with a T3 promoter), the system prevents the production of double-stranded RNA (dsRNA) and eliminates transcriptional “clash” or interference.

Sequential Mapping and Readout: The inclusion of an SP6 promoter allows for the initial mapping of insertion sites and barcode associations from genomic DNA via in vitro transcription (IVT). Subsequent rearrangement identities are determined following recombination using T7 or T3-driven IVT, which can be analyzed via bulk amplicon sequencing or single-cell RNA sequencing (scRNA-seq) from fixed mammalian cells.

The system has been successfully implemented and validated in multiple mammalian cell types, including HEK293T cells and mouse embryonic stem cells (mESCs). All three phage promoters (T7, T3, and SP6) are compatible with in situ transcription (IST) from fixed mammalian cells (36). In one example, the system employs a split Blasticidin resistance (BsdR) marker driven by a constitutive PGK promoter. In another example, a split GFP marker is utilized, driven by an inducible tetracycline-responsive promoter (TRE). In this example, the required transactivator (rtTA) is provided either in trans via transient transfection or via a stable, genomically integrated expression cassette.

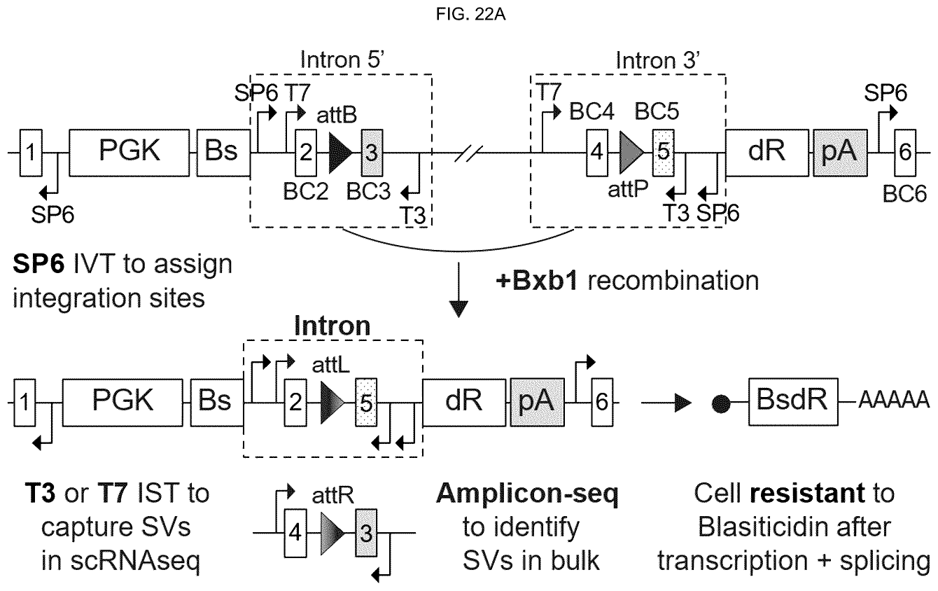

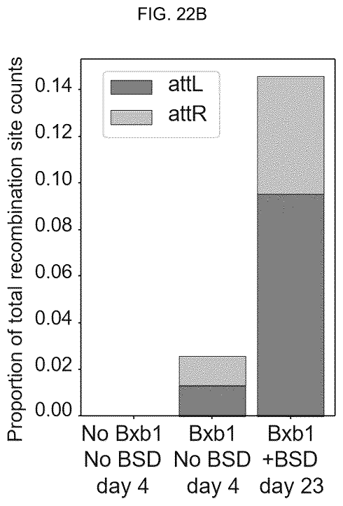

The efficacy of the system was demonstrated in an experiment where half-constructs containing Bxb1 recombination sites and barcodes within synthetic introns were delivered to host cells via PiggyBac transposition at a high multiplicity of infection (MOI) (40). Following the expression of Bxb1 recombinase, cells were treated with Blasticidin. The selection resulted in a robust enrichment of SV-containing cells with minimal background, as evidenced by the lack of survivors in the absence of the recombinase. Bulk amplicon sequencing on genomic DNA revealed about a 5-fold increase in the fraction of reads, indicating induced SVs rising from approximately 3% at day 4 (immediately post-induction) to 15% at day 23 (post-selection) (FIG. 22B).

This represents a significant improvement over prior methodologies, where SV-containing cells often remained undetectable by day 7. Mapping of the unique barcodes to their original integration sites confirmed that the enriched population contained a diverse array of SV types, including deletions, inversions, and chromosomal translocations, as well as ecDNAs. The system is demonstrated to have the capacity for comprehensive functional characterization of the structural genome.

Data/Code Availability. Analysis pipelines and plasmid sequences are available online at: github.com/sudpinglay/genome-shuffle-seq

Raw sequencing data and processed files are available online at: krishna.gs.washington.edu/content/members/pinglay/sud/genome_shuffle_geo/

Raw sequencing data and processed data files supporting the present disclosure have been deposited in the Gene Expression Omnibus (GEO) under accession number GSE282636. The contents of the GEO deposit are hereby incorporated by reference in their entirety for all purposes.

The methodologies described herein may be implemented using one or more computing devices. These devices may be implemented as one or more server computers, a network element on dedicated hardware, a software instance running on dedicated hardware, or as a virtualized function instantiated on a cloud infrastructure. In some embodiments, the computing system is implemented as a plurality of devices with components and data distributed among them.

The computing system comprises at least one memory and one or more processors. The memory may be volatile (such as RAM), non-volatile (such as ROM or flash memory), or a combination thereof. It is configured to store various components, including objects, modules, and instructions (e.g., custom Python scripts) to perform the genomic analysis functions disclosed herein. One or more processors are configured to execute the instructions stored in the memory to perform data processing tasks.

The computing device includes additional storage, which may be removable or non-removable, including magnetic disks, optical disks, or tape storage. The memory and storage are examples of computer-readable media, which may include computer storage media and communication media. Computer storage media include volatile and non-volatile, removable and non-removable media implemented in any method or technology for the storage of information, such as computer-readable instructions, data structures, program modules, or other data. The system may include input devices (e.g., a keypad, touch-sensitive display, or voice input) and output devices (e.g., a display or printer). In particular embodiments, the input device includes a device configured to capture genomic or medical images, or a sequencing platform configured to provide raw sequence reads for analysis. The computing system further includes one or more wired or wireless transceivers, such as a Network Interface Card (NIC) or a Local Area Network (LAN) adapter. These transceivers facilitate the connection to various networks and servers for the deposition and retrieval of data, such as the deposition of raw sequencing data and processed files into public databases.

### (VI) SELECTED REFERENCES

1. Mills et al., Nature 470, 59-65 (2011).
2. 1000 Genomes Project Consortium, Auton et al., Nature 526, 68-74 (2015).
3. Belyeu et al., Am. J. Hum. Genet. 108, 597-607 (2021).
4. Weischenfeldt et al., Nat. Rev. Genet. 14, 125-138 (2013).
5. Chiang et al., Nat. Genet. 49, 692-699 (2017).
6. Fudenberg et al., Proc. Natl. Acad. Sci. U.S.A 116, 2175-2180 (2019).
7. Collins et al., Nature 581, 444-451 (2020).
8. Shendure and Akey, Science 349, 1478-1483 (2015).
9. Cuella-Martin et al., Cell 184, 1081-1097.e19 (2021).
10. Kircher et al., Nat. Commun. 10, 3583 (2019).
11. Findlay et al., Nature 562, 217-222 (2018).
12. Takahashi et al., Science 264, 1724-1733 (1994).
13. Bauer et al., Science 342, 253-257 (2013).
14. Schmidt et al., Nature, doi: 10.1038/s41586-023-06835-6 (2023).
15. Ovcharenko et al., Genome Res. 15, 137-145 (2005).
16. Nóbrega et al., Nature 431, 988-993 (2004).
17. Leypold and Speicher, Trends Genet. 37, 903-918 (2021).
18. Real et al., Science 370, 208-214 (2020).
19. Kapusta et al., Proc. Natl. Acad. Sci. U.S.A 114, E1460-E1469 (2017).
20. Mouse Genome Sequencing Consortium, Waterston et al., Nature 420, 520-562 (2002).
21. Noer et al., Trends Genet. 38, 766-781 (2022).
22. Leibowitz et al., Annu. Rev. Genet. 49, 183-211 (2015).
23. Pradella et al., bioRxiv, doi: 10.1101/2023.06.25.546239 (2023).
24. Mills et al., Trends Genet. 17, 331-339 (2001).
25. Zheng et al., Mol. Cell. Biol. 20, 648-655 (2000).
26. Kraft et al., Cell Rep. 10, 833-839 (2015).
27. Boroviak et al., Sci. Rep. 7, 12867 (2017).
28. Liu et al., Nucleic Acids Res. 50, 3456-3474 (2022).
29. Bilodeau et al., Nat. Methods 4, 263-268 (2007).
30. Fortier et al., PLOS Genet. 6, e1001241 (2010).
31. Dymond et al., Nature 477, 471-476 (2011).
32. Zhou et al., Natl Sci Rev 10, nwad073 (2023).
33. Shen et al., Genome Res. 26, 36-49 (2016).
34. Hoess et al., Nucleic Acids Res. 14, 2287-2300 (1986).
35. Replogle et al., Nat. Biotechnol. 38, 954-961 (2020).
36. Askary et al., Nat. Biotechnol. 38, 66-75 (2020).
37. Li et al., Cell 187, 2411-2427.e25 (2024).
38. Eckersley-Maslin et al., Dev. Cell 28, 351-365 (2014).
39. Keane et al., Nature 477, 289-294 (2011).
40. Kalhor et al., Science 361 (2018).
41. Siebert et al., Nucleic Acids Res. 23, 1087-1088 (1995).
42. Plessy et al., Nat. Methods 7, 528-534 (2010).
43. Loonstra et al., Proc. Natl. Acad. Sci. U.S.A 98, 9209-9214 (2001).
44. Kurachi et al., Immunity 51, 591-592 (2019).
45. Zhu et al., Genesis 50, 102-111 (2012).
46. Matsuda et al., Proc. Natl. Acad. Sci. U.S.A 104, 1027-1032 (2007).
47. Tichy et al., Stem Cell Res. 10, 428-441 (2013).
48. Ayaz et al., Stem Cells 40, 883-891 (2022).
49. Xu et al., BMC Biotechnol. 13, 87 (2013).
50. Jelicic et al., Nucleic Acids Res. 51, 5285-5297 (2023).
51. Jusiak et al., ACS Synth. Biol. 8, 16-24 (2019).
52. Barra et al., Nat. Commun. 9, 4340 (2018).
53. Datlinger et al., Nat. Methods 18, 635-642 (2021).
54. Martin et al., Nat. Protoc. 18, 188-207 (2023).
55. Sziraki et al., Nat. Genet. 55, 2104-2116 (2023).
56. Dixit et al., Cell 167, 1853-1866.e17 (2016).
57. Datlinger et al., Nat. Methods 14, 297-301 (2017).
58. Pinglay et al., Science 377, eabk2820 (2022).
59. Koeppel et al., bioRxiv (2024) p. 2024.01.22.576745.
60. Xu et al., Nat. Commun. 14, 1984 (2023).
61. Fishilevich et al., Database 2017 (2017).
62. Luo et al., Nucleic Acids Res. 48, D882-D889 (2020).
63. Lalanne et al., Nat. Methods 21, 983-993 (2024).
64. Yusa et al., Proc. Natl. Acad. Sci. U.S.A 108, 1531-1536 (2011).
65. Brosh et al., Proc. Natl. Acad. Sci. U.S.A 118 (2021).
66. Martin, EMBnet.journal 17, 10-12 (2011).
67. Li and Durbin, Bioinformatics 25, 1754-1760 (2009).
68. Li et al., Bioinformatics 25, 2078-2079 (2009).
69. Neph et al., Bioinformatics 28, 1919-1920 (2012).
70. Quinlan and Hall, Bioinformatics 26, 841-842 (2010).
71. Robinson et al., Nat. Biotechnol. 29, 24-26 (2011).
72. Yu et al., Bioinformatics 31, 2382-2383 (2015).
73. Creators Mori Hideto Max Base1 Daniel Bullock2 bw2 morityun snajder-r Show affiliations 1. @GitHub Open Source Maintainer 2. University of Minnesota, ponnhide/pyCircos: pyCircos: Circos Plot in Matplotlib (https://zenodo.org/doi/10.5281/zenodo.6477640).
74. Hao et al., Cell 184, 3573-3587.e29 (2021).
75. Wolock et al., Cell Syst 8, 281-291.e9 (2019).
76. Wang et al., BMC Genomics 23, 135 (2022).
77. Ribeiro-Dos-Santos et al., Genome Res. 32, 425-436 (2022).
78. Wolf et al., Genome Biol. 19, 15 (2018).
79. Frankish et al., Nucleic Acids Res. 49, D916-D923 (2021).
80. Ramírez-Solis et al., Nature 378, 720-724 (1995).
81. Conrad et al., Commun. Biol. 3, 439 (2020).
82. Ross et al., Synthetic Biology (2025).
83. Callen et al., Mol. Cell 14, 647-656 (2004).
84. Jillette et al., Nat Commun 10, 4968 (2019).

### (VII) CLOSING PARAGRAPHS

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A shuffle cassette comprising a recombination site, a pair of barcodes comprising a first barcode and a second barcode flanking the recombination site, a first primer binding site, a second primer binding site, a first promoter, and a second promoter, wherein the first barcode has a different sequence compared to the second barcode.

2. The shuffle cassette of claim 1, wherein the recombination site comprises IoxPsym, IoxP, attB/attP, FRT, Rox, attL/attR, att, Gix, a synthetic recombination site, or an RNA recombination site.

3. The shuffle cassette of claim 1, wherein the first barcode or the second barcode comprises 2 to 50 nucleotides in length.

4. The shuffle cassette of claim 1, wherein the first primer binding site or the second primer binding site comprises the sequence as set forth in SEQ ID NO: 1 or SEQ ID NO: 2.

5. The shuffle cassette of claim 1, wherein the first promoter or the second promoter comprises an inducible promoter that is inert in a host cell.

6. The shuffle cassette of claim 1, wherein the first promoter or the second promoter comprises a T7 promoter, a T3 promoter, or an SP6 promoter.

7. The shuffle cassette of claim 1, further comprising a payload comprising a selectable marker, a detectable marker, or a splitable marker.

8. A vector comprising the shuffle cassette of claim 1.

9. The vector of claim 8, wherein the vector is a transposon vector, a viral vector, or a targeted gene editing vector.

10. The vector of claim 9, wherein the transposon vector comprises a PiggyBac transposon vector, a Sleeping Beauty transposon vector, Tol2 transposon vector, Mariner transposon vector, Frog Prince transposon vector, TcBuster transposon vector, or SpinON transposon vector.

11. A shuffle cassette library comprising a plurality of the shuffle cassettes of claim 1, wherein each of the shuffle cassettes comprises a different first barcode and each of the shuffle cassettes comprises a different second barcode, and wherein the first barcode of each of the shuffle cassettes is different from the second barcode within the same shuffle cassette.

12. A cell library comprising the shuffle cassette library of claim 11.

13. A method comprising:
introducing a plurality of the shuffle cassettes of claim 1 into a genome of a host cell; and
providing to the host cell a recombinase targeting the recombination site of the shuffle cassettes,
thereby inducing recombination between the shuffle cassettes introduced in the genome of the host cell to generate a structural variant and a set of barcode combinations, the set of barcode combinations comprising the barcodes from shuffle cassettes introduced on a same chromosome in a same orientation indicative of a deletion or in opposite orientations indicative of an inversion.

14. The method of claim 13, wherein the introducing comprises transposition or retrotransposition.

15. The method of claim 13, wherein the providing the recombinase comprises administering a plasmid encoding the recombinase.

16. The method of claim 13, further comprising mapping positions and/or orientations of the shuffle cassettes within the host cell's genome before the providing.

17. The method of claim 16, wherein the mapping comprises:
providing a polymerase that binds the first and/or second promoter of each of the shuffle cassettes;
transcribing, using the polymerase, a transcript comprising shuffle cassette-specific barcodes and genomic sequences flanking each of the shuffle cassettes;
sequencing the transcript; and
associating each pair of barcodes within each of the shuffle cassettes with a genomic coordinate based on the genomic sequences flanking each of the shuffle cassettes.

18. The method of claim 13, further comprising:
determining an identity of the structural variant by decoding the set of barcode combinations; and
sequencing the structural variant,
wherein the structural variant comprises deletions, inversions, chromosomal translocations, or extrachromosomal circular DNA.

19. The method of claim 13, further comprising genotyping the host cell after the providing the recombinase, the genotyping comprising single-cell RNA sequencing (scRNAseq), bulk amplicon sequencing, in vitro transcription sequencing (IVT-seq), in situ transcription sequencing (IST-seq), or performing polymerase chain reaction (PCR).

20. The method of claim 13, further comprising in situ transcription sequencing followed by single-cell RNA sequencing after the providing the recombinase, thereby capturing an identity of the structural variant and transcriptomic data from the host cell, wherein the structural variant comprises deletions, inversions, chromosomal translocations, or extrachromosomal circular DNA.

21. The method of claim 14, wherein the host cell comprises a mammalian cell, a plant cell, an insect cell, a bacterial cell, a fungal cell, a protozoan cell, a fish cell, an amphibian cell, a reptilian cell, or an avian cell.

22. A method comprising:
introducing at least two different shuffle cassettes into a genome of a host cell, each of the shuffle cassettes comprising a recombination site, a pair of barcodes flanking the recombination site, at least two promoters, at least one primer binding site, and a half of a splitable marker, wherein the recombination site, the barcodes, and the promoters are embedded within a synthetic intron;
providing a recombinase targeting the recombination site of each of shuffle cassettes to the host cell; and
reconstituting the at least two shuffle cassettes into a recombinant construct,
thereby inducing recombination of the recombinant construct introduced in the genome of the host cell to generate a structural variant.

23. The method of claim 22, wherein the splitable marker comprises a split Blasticidin resistance (BsdR) marker, a split green fluorescent protein (GFP) marker, or a split red fluorescent protein (RFP) marker.

24. The method of claim 22, wherein the at least two promoters comprise a phosphoglycerate kinase (PGK) promoter or a tetracycline-responsive promoter (TRE).

25. A kit comprising the shuffle cassette of claim 1, further comprising a recombinase comprising Cre recombinase, Bxb1 integrase, FLP recombinase, A phage integrase, PhiC31 integrase, Dre recombinase, TP901-1 integrase, or Gin recombinase.


SEQUENCE LISTING (4 sequences)

SEQ ID NO: 1 - DNA, 22 bp, synthetic construct
  source 1..22: mol_type = other DNA
gctttaaggccggtcctagcaa

SEQ ID NO: 2 - DNA, 22 bp, synthetic construct
  source 1..22: mol_type = other DNA
gctcacctattagcggctaagg

SEQ ID NO: 3 - DNA, 18 bp, synthetic construct
  source 1..18: mol_type = other DNA
taatacgactcactatag

SEQ ID NO: 4 - DNA, 13 bp, synthetic construct
  source 1..13: mol_type = other DNA
ccctagaaagata